SEQUENCE LISTING (34 sequences)

SEQ ID NO: 1 - DNA, 418 bp, Hirudo medicinalis
  CDS (1)..(246)
atggctcttctcaacaagttgctctgctttgctctggtcttcatgatcttcggtgagttcgtgactccggattgctacgaggactggagcaggtgcacgcctggaacgtcattcctgactggaatcctgtggaaagattgccacagccgttgcaaggaactcggtcacaggggaggacgatgtgtggattcaccaagcaaacactgccctggagtcctcaagaacaacaaacagtgtcattgctactgatctgaagaattgttgaaatatgttggagattctggaaggacagtgaccatgatttgtgattttctttaaaaatcacttagttaagtaaaaacattggcgatgattacgcttacataataaagtattattttgcgattgcaccattccaaaaaaaaaaaaaaaaaaaaaa

SEQ ID NO: 2 - protein, 82 aa, Hirudo medicinalis
MALLNKLLCFALVFMIFGEFVTPDCYEDWSRCTPGTSFLTGILWKDCHSRCKELGHRGGRCVDSPSKHCPGVLKNNKQCHCY

SEQ ID NO: 3 - DNA, 414 bp, Hirudo medicinalis
  CDS (1)..(171)
atgttcagcaagtacgagaggcaaaaggacaaaagaagttacggtgaaaggttcagcatgttcacaggccctcagttcatttcgccccccgagaggatcaaacccaacaaaattctccagtgggatggggagggcatgcccatttacgccacgtcgggggctgcagccgagtaaggagagccgattggcgactgaccagccaatatgaccaggcaatgacaagccaggatatttttggagtaattcgacattcagagacggtccttaaaatgtctccaatgatttgtggaatcgattcgagttttgaattaaacgatatctcaataaaatatcggttaaatttttgcaatttcgtcaccaaatattaatgtcaaataaatagttctttctaaagcgaaaaaaaaaaaaaaaaaa

SEQ ID NO: 4 - protein, 57 aa, Hirudo medicinalis
MFSKYERQKDKRSYGERFSMFTGPQFISPPERIKPNKILQWDGEGMPIYATSGAAAE

SEQ ID NO: 5 - DNA, 398 bp, Theromyzon tessulatum
  CDS (1)..(294)
atggaattgaaatctggtctcagtattttgttgtgctttgggatctgcattgcagtgattaatgcgggatgtttcgaagattggagtcgttgttcgccatcgacgtctcgtggaacaggagttttatggagagattgtgacagttactgcaaagtttgtttcaaagctgataggggagaatgttttgattcaccaagtctaaattgtccacaacgtctaccaaataacaaacaatgcaggtgcataaacgctagaactgcaaaagacaataggaatccaacttgttgggcttaactttaatcagaaaaaaacaatgcttttataatttattttgttaaactttattgcgctcattttttatctgaaaataaaacatgtatgaattaaaaaaaaaaaaa

SEQ ID NO: 6 - protein, 97 aa, Theromyzon tessulatum
MELKSGLSILLCFGICIAVINAGCFEDWSRCSPSTSRGTGVLWRDCDSYCKVCFKADRGECFDSPSLNCPQRLPNNKQCRCINARTAKDNRNPTCWA

SEQ ID NO: 7 - DNA, 225 bp, Hirudo medicinalis
  CDS (1)..(225)
tgtttcgaagattggagtcgttgttcgccatcgacagctagtgcaacaggagttttatggagaagttgtgacagttactgcaaagtttgtttcaaagctgataggggagaatgttatgattcgccaagtctcaattgtccacatcgtctaccaaataacaaacaatgcaggtgcataaacgctagaactgcaaaagacaataggaatccaacttgttgggcttga

SEQ ID NO: 8 - protein, 74 aa, Hirudo medicinalis
CFEDWSRCSPSTASATGVLWRSCDSYCKVCFKADRGECYDSPSLNCPHRLPNNKQCRCINARTAKDNRNPTCWA

SEQ ID NO: 9 - DNA, 633 bp, Aplysia californica
  CDS (200)..(430)
ggctcgagcggctcgagtgacgccagcactagatctatatgtatccaacaaagtctcgacatcttgcagttgcagttccccaagaaggcctatttaatcgacggttggaacttttccttccgcccagctccttgaaaagaggcagccagggaacataacaagaagattgtcattagaaactgctgggacacttggcacgatggacaagaaggcagcaaatggaggaaaagagaagggccccttggaagcctgctgggacgagtggagcagatgtacaggttggagctctgctggcactggagttctttggaaatcttgtgatgaccagtgcaaaaagctggggaaaagcggtggagaatgtgtgctcactccctctacttgcccatttacacgtaccgacaaggcttaccaatgccagtgcaagaagtaaagttttacacaggagctgacgcgtccacgattatctacttcgtcgggaccacatctagtgaggcaattggtgcaaacatattgacataggtttattctttccgggaactcagaaattcagaattttgaagtttaattaagtaaacaacacgttagataatgggatttcaaaaataaagatgaaattatgaagttttaaaaaaa

SEQ ID NO: 10 - protein, 76 aa, Aplysia californica
MDKKAANGGKEKGPLEACWDEWSRCTGWSSAGTGVLWKSCDDQCKKLGKSGGECVLTPSTCPFTRTDKAYQCQCKK

SEQ ID NO: 11 - DNA, 555 bp, Lumbricus rubellus
  CDS (84)..(314)
ggcacgagctatctctctgcctcatcactctccctttcccctctctctctctcattatcttgctgtctctctgtctgactatcatgtctctctgtatctctgactatctctatctgactctgactttctcaaagtacgaacgccagaaggacaagaggccatactcggaacgcaagaaccaatacacgggtccgcagttcctctatcctccggagcgcatcccaccgcagaaggtcatcaaatggaacgaggagggtcttcccatctacgaaatccccggcgaaggaggtcacgcagaaccagctgccgcctaggttagatttcagctgaaccgattgccaaccggagaggaagagagttgatttcgatagagcgtgtggacagaactatcagcgttctttttaccatcgtcgctataagtctatcactcttagaggatcaagtagattgcgtagacctagttaactaaacctaaatcaattgttgtcttggttttaaatgagtggagaggaaaattaaacaaattacaacccctaaaaaaaaaaaaaaaaaaaa

SEQ ID NO: 12 - protein, 76 aa, Lumbricus rubellus
MSLCISDYLYLTLTFSKYERQKDKRPYSERKNQYTGPQFLYPPERIPPQKVIKWNEEGLPIYEIPGEGGHAEPAAA

SEQ ID NO: 13 - DNA, 622 bp, Pheretima tschiliensis
  CDS (43)..(246)
aaaattcaggagcgctttgcaggaactccggcaaagatcgagatgtacagcaagtacgagagacagaaggacaagagaccgtactcggagcggaaggaccagtacaccggaccgcagttcctctatccgcccgatcgcatcccgccgagcaaagccatcaaatggaacgaagagggccttccgatgtacgaggtcctcccggacggagcaggggcaaagaccgccgttgaggccgccgctgaatagaatatttgaccgattaaacgctctagacattcggatgatttgagatgcaacatgacgcctgcgatcacagagtgaccttgacagtaacctttcgtaggaaccgacaaatcgctgctaataaacactggcgcctcataaattctaaaatcctgagtagaaaccgatgtaatctagtggctccgttcgattagtttagcaattactttagcgcacttatctgaatgccgtcagtcgatggaacaaattagttgattttgcagatttctaagaacaacaacgaagtcgatttctgtatcggaattacctttgatctatttagaacagccactgcctctaattcgagaattcaataaaatccagcagttttagcagaatg

SEQ ID NO: 14 - protein, 67 aa, Pheretima tschiliensis
MYSKYERQKDKRPYSERKDQYTGPQFLYPPDRIPPSKAIKWNEEGLPMYEVLPDGAGAKTAVEAAAE

SEQ ID NO: 15 - protein, 57 aa, Lumbricus rubellus
GCYEDWSRCTPSTSWLTGILWKSCTNRCKEQGHRGGNCRDSPSPCPGLQNNKQCYCF

SEQ ID NO: 16 - protein, 62 aa, Biomphalaria glabrata
NVIGRCWDTWSRCSTWSRWFTGRVWLTRDGKCRELGKRGGNCVMTPSTCPLSSEAFQCQCYT

SEQ ID NO: 17 - DNA, 25 bp, Artificial Sequence
  amorce
atggaggaggaaattgaagaactcc

SEQ ID NO: 18 - DNA, 35 bp, Artificial Sequence
  Amorce
cgagtcgacatcgatcgtttttttttttttttttt

SEQ ID NO: 19 - DNA, 18 bp, Artificial Sequence
  Amorce
gantgntangangantgg

SEQ ID NO: 20 - DNA, 24 bp, Artificial Sequence
  Amorce
cagtcaggaatgacgttccaggcg

SEQ ID NO: 21 - DNA, 18 bp, Artificial Sequence
  Amorce
tcccctgtgaccgagttc

SEQ ID NO: 22 - DNA, 20 bp, Artificial Sequence
  Amorce
ccaatcggctctccttactc

SEQ ID NO: 23 - DNA, 20 bp, Artificial Sequence
  Amorce
acaggccctcagttcatttc

SEQ ID NO: 24 - DNA, 20 bp, Artificial Sequence
  Amorce
gtggacgcctggaacgtcat

SEQ ID NO: 25 - DNA, 20 bp, Artificial Sequence
  Amorce
cttgaggactccagggcagt

SEQ ID NO: 26 - DNA, 20 bp, Artificial Sequence
  Amorce
tgcggttatttcgattgtca

SEQ ID NO: 27 - DNA, 20 bp, Artificial Sequence
  Amorce
agacaaatcgctccaccaac

SEQ ID NO: 28 - protein, 59 aa, Hirudo medicinalis
DCYEDWSRCTPGTSFLTGILWKDCHSRCKELGHRGGRCVDSPSKHCPGVLKNNKQCHCY

SEQ ID NO: 29 - protein, 56 aa, Artificial Sequence
  conserved cysteine pattern
CXXXXXXCXXXXXXXXXXXXXXCXXXCXXXXXXXXXCXXXXXXXCXXXXXXXXCXC

SEQ ID NO: 30 - protein, 57 aa, Artificial Sequence
  conserved cysteine pattern
CXXXXXXCXXXXXXXXXXXXXXCXXXCXXXXXXXXXXCXXXXXXCXXXXXXXXXCXC

SEQ ID NO: 31 - protein, 58 aa, Artificial Sequence
  conserved cysteien pattern
CXXXXXXCXXXXXXXXXXXXXXCXXXCXXXXXXXXXXCXXXXXXCXXXXXXXXXXCXC

SEQ ID NO: 32 - protein, 56 aa, Artificial Sequence; identical to SEQ ID NO: 29
  conserved cysteien pattern

SEQ ID NO: 33 - protein, 57 aa, Artificial Sequence
  conserved cystein pattern
CXXXXXXCXXXXXXXXXXXXXXCXXXCXXXXXXXXXCXXXXXXXCXXXXXXXXXCXC

SEQ ID NO: 34 - protein, 57 aa, Artificial Sequence
  conserved cysteine pattern
CXXXXXXCXXXXXXXXXXXXXXCXXXCXXXXXXXXXCXXXXXXCXXXXXXXXXXCXC

(19) United States
(12) Patent Application Publication
(10) Pub. No.: US 2010/0136034 A1
(43) Pub. Date: Jun. 3, 2010

(54) NOVEL MULTIMERIC MOLECULES, A PROCESS FOR PREPARING THE SAME AND THE USE THEREOF FOR MANUFACTURING MEDICINAL DRUGS

(75) Inventors: Gilles Guichard, Gradignan (FR); Sylvie Fournel, Strasbourg (FR); Nathalie Trouche, Krafft (FR); Sebastien Wieckowski, Ambilly (FR)

Correspondence Address: YOUNG & THOMPSON, 209 Madison Street, Suite 500, Alexandria, VA, 22314, US

(73) Assignee: CENTRE NATIONAL DE LA RECHERCHE SCIENTIFIQUE, Paris Cedex 16 (FR)

(21) Appl. No.: 12/525,761

(22) PCT Filed: Feb. 4, 2008
(86) PCT No.: PCT/FR2008/000129

§ 371 Date: Jan. 22, 2010

(30) Foreign Application Priority Data
Feb. 5, 2007 (FR) 0700809

(51) Int. Cl. A61K 38/12; C07K 7/64; A61K 39/00; A61P 37/00; A61P 29/00

(52) U.S. Cl. 424/184.1; 530/317; 514/11

(57) ABSTRACT

The invention relates to a compound of the formula (I):

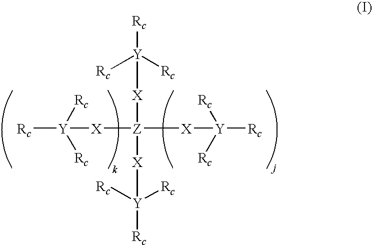

in which k and j are independently 0 or 1, Y is a macrocycle in which the cycle includes 9 to 36 carbon atoms and is functionalised by three amino functions and by a chain for attaching the spacer arm Z via an X bond, R_{c} is a binding pattern with a receptor of the TNF superfamily, X is a chemical function for binding the Y group to the space arm, and Z is a bi-, tri- or tetra-functional spacer arm.

The invention is directed to novel multimeric molecules, to a process for preparing the same and to the use thereof for manufacturing medicinal drugs.

The invention is further directed to molecules capable of stimulating or inhibiting the immune response.

The important role of the CD40/CD40L pair in the immune response has led a great number of research teams to use antibodies raised against both to such molecules for therapeutic ends so as to inhibit or stimulate the immune system. Administering anti-CD40L antibodies has given promising results in the treatment of autoimmune disorders such as experimental murine allergic encephalomyelitis (a model for human multiple sclerosis) (Howard et al.; 1999) or in the treatment of kidney allogenic graft rejection in monkeys (Kirk et al, 1999). In both cases, antibodies inhibited an adverse activity of the immune system. By contrast, the use of agonistic anti-CD40 antibodies has resulted on one hand in a strong improvement of the response to antitumour peptide vaccines in mice (Diehl et al., 1999) and on the other, in enhancement of the CD₄⁺ T cell efficiency in murine tumour control (Sotomayor et al., 1999; Lode et al., 2000). Tumour regression in murine models was demonstrated following injection of dendritic cells (CDs) transformed by an adenovirus coding for CD40L (Kikuchi et al., 2000). Finally, activation of dendritic cells through interaction of their CD40 molecules with CD40L is capable of protecting mice from a parasitic infection, Trypanosoma Cruzi (Chaussabel et al., 1999). Throughout the research work accomplished so far, the specific valency of the CD40L molecule which self-combines in the form of trimers to form hexavalent complexes with CD40 complicates the production of functional antibodies capable of interfering with the CD40/CD40L pair at the functional level. Development of adenoviruses coding for CD40L has in part overcome this drawback. However, their use is not without hazard for man. Finally, the specific valency of the system stands as an obstacle against the discovery of synthetic molecules which can interfere with ways CD40/CD40L interact.

The invention is aimed at providing multimeric ligands designed to interfere with protein-protein interactions.

The present invention is also aimed at preparing molecules capable of interfering with multivalent protein-protein interactions.

The present invention is further aimed at preparing molecules capable of fine tuning or modulating the activity of members belonging to the TNF and TNF-R family.

The present invention is aimed at providing a synthetic molecule which acts on the CD40/CD40L system.

The present invention relates to a compound having the following formula (I):

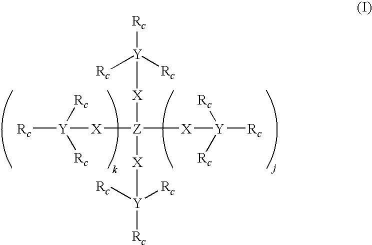

wherein:
k and j denote independently from each other 0 or 1,
Y denotes a macrocycle the ring of which includes from 9 to 36 atoms, and is functionally substituted by three amine functionalities allowing binding of R_{c} via its C-terminal carboxyl functional group and by a carbon chain allowing binding of a Z spacer via an X bond,
R_{c} denotes a binding motif to a receptor belonging to the TNF superfamily, and preferably stands for a ligand derived-sequence selected among the residues interfacing with the ligand receptor, which sequence may interact with the receptor, said ligand being selected among receptor ligands belonging to the TNF superfamily, namely among the following ligands: EDA, CD40L, FasL, OX40L, AITRL, CD30L, VEGI, LIGHT, 4-1BBL, CD27L, LTα, TNF, LTβ, TWEAK, APRIL, BLYS, RANKL and TRAIL,
X denotes a chemical functionality which allows the Y group to be linked to the spacer and is selected among the following functional groups:

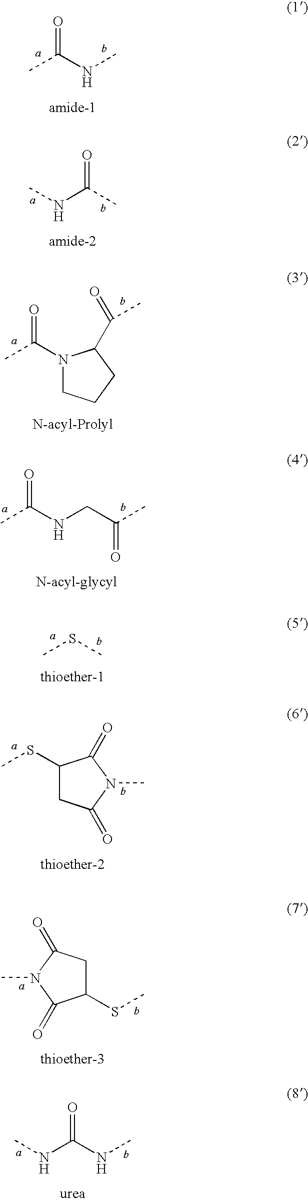

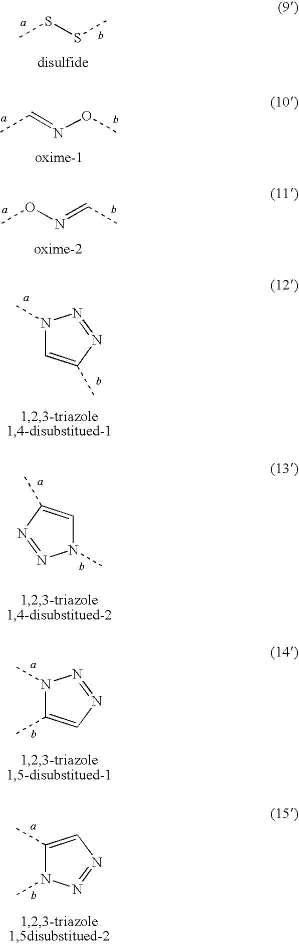

a designating the bond to the Y group and b designating the bond to the Z group,
Z denotes a bi, tri- or tetrafunctional spacer allowing dimerization, trimerization or tetramerization of

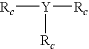

via formation of an X type bond, the Z spacers having one of the following formulae:

if j=k=0
if X stands for a group of formulae (1′), (8′), (9′), (5′), (6′), (7′), (13′) and (15′), Z has one of the following formulae:

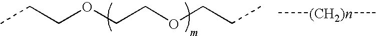

m being an integer ranging from 1 to 40,

n being an integer ranging from 1 to 10,
if X stands for a group of formulae (2′), (3′) and (4′), Z has one of the following formulae:—

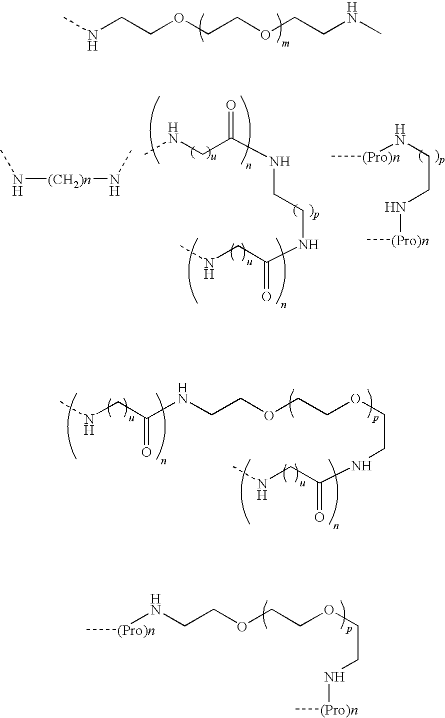

if X stands for a group of formulae (12′) and (14′), Z has one of the following formulae:—

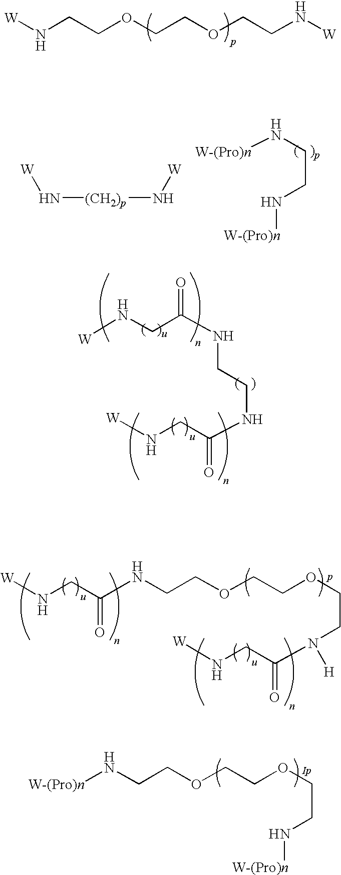

m and n being as defined above,

p being an integer ranging from 1 to 6,

u being an integer ranging from 1 to 4,

W designating a group of formula

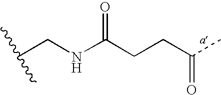

or a group of formula

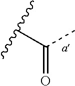

the W group being bound to the NH group through the dotted line bond a′,
if X stands for a group of formulae (1′), (2′), (8′), (9′), (5′), (6′), (7′), (10′), (11′), (13′) and (15′), Z has one of the following formulae:—

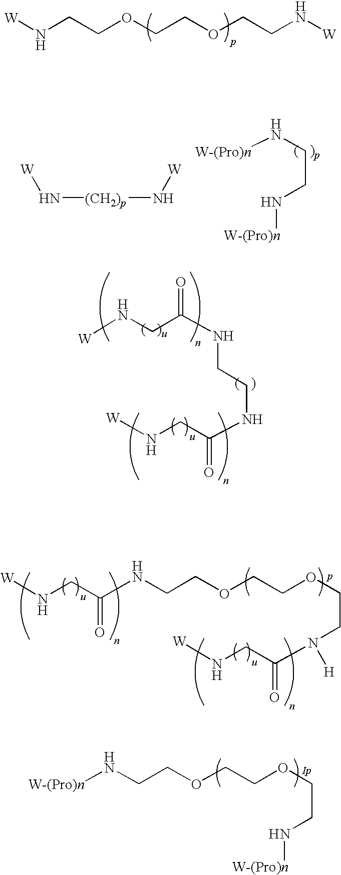

m and n being as defined above,

p being an integer ranging from 1 to 6,

u being an integer ranging from 1 to 4,

W designating a group of formula

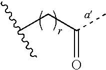

r being an integer ranging from 1 to 4,

the W group being bound to the NH group via the dotted line bond a′

if j=1 or k=1
if X stands for a group of formulae (12′) and (14′), Z has one of the following formulae:—

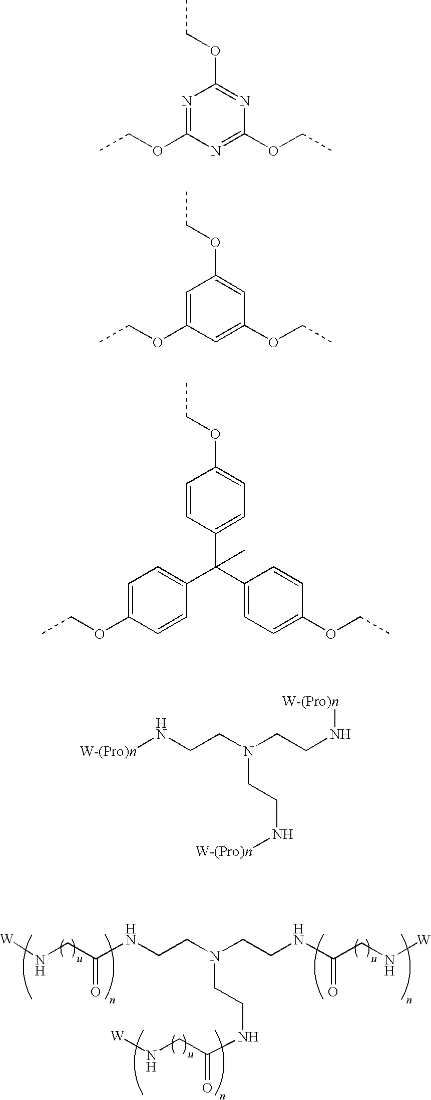

u being an integer ranging from 1 to 4,

n being an integer ranging from 1 to 10,

W designating a group of formula

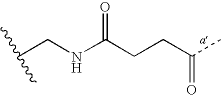

or a group of formula

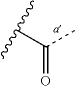

the W group being bound to the NH group through the dotted line bond a′,
if X denotes a group of formulae (1′), (2′), (8′), (9′), (5′), (6′), (7′), (10′), (11′), (13′) and (15′), Z has one of the following formulae:

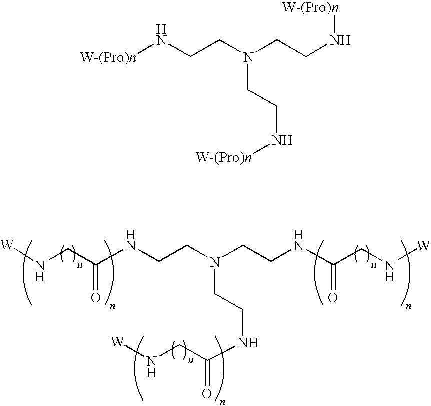

u being an integer ranging from 1 to 4,

n being an integer ranging from 1 to 10,

W designating a group of formula
r being an integer ranging from 1 to 4,

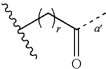

the W group being bound to the NH group via the dotted line bond a′
Z can also denote one of the following formulae:

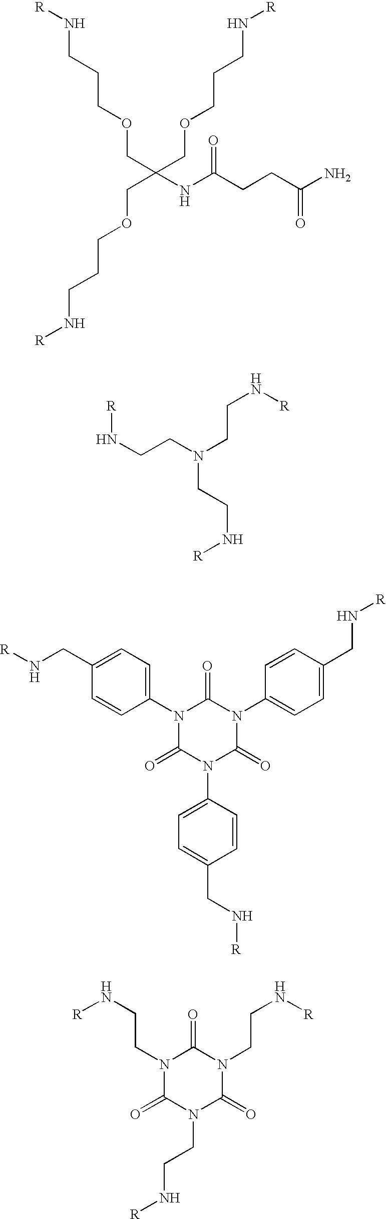

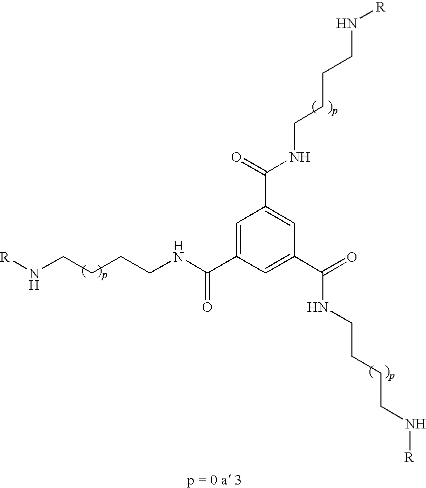

wherein:
   if X denotes a group of formulae (1′), (8′), (9′), (5′), (6′), (7′), (13′) and (15′):
   R stands for one of the following groups:

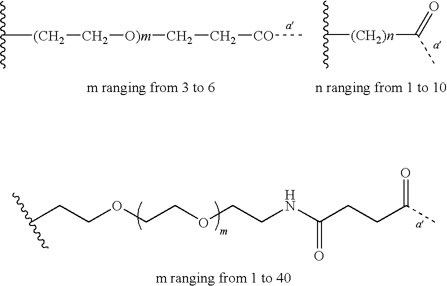

the R group being bound to the NH group via the dotted line bond a′,
if X denotes a group of formulae (2′), (3′) and (4′):
R stands for one of the following groups:

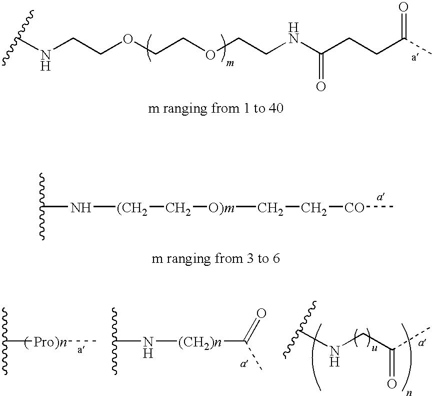

n being an integer ranging from 1 to 10,

u being an integer ranging from 1 to 4,

the R group being bound to the NH group through the dotted line bond a′,
where X denotes a group of formulae (12′) and (14′):
R stands for one of the following groups:

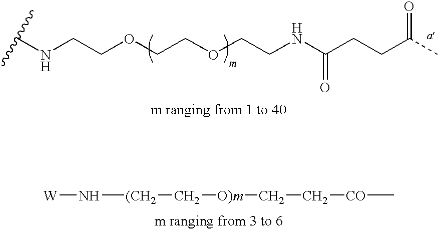

the R group being bound to the NH group through the dotted line bond a′,

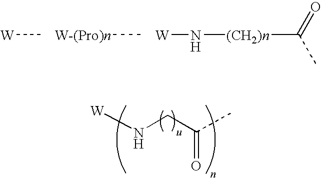

n designating an integer ranging from 1 to 10,

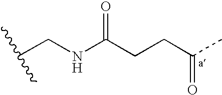

W designating a group of formula

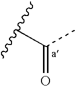

or a group of formula

the W group being bound to the NH group through the dotted line bond a′,
if X denotes a group of formulae (1′), (2′), (8′), (9′), (5′), (6′), (7′), (10′), (11′), (13′) and (15′):

R designates one of the following groups:

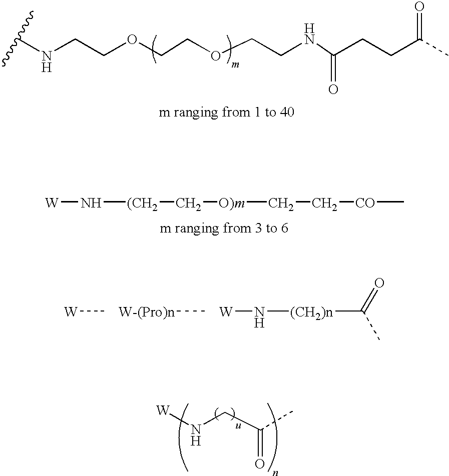

u and n being as specified above,

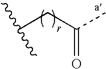

W designating a group of formula
r being an integer ranging from 1 to 4,

the W group being bound to the NH group through the dotted line bond a′.

Hence, the present invention deals with dimers (when j=k=0), trimers (when j=1 or k=1) and tetramers (when j=k=1) of the

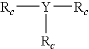

motif.

According to an advantageous mode of execution, the compounds of the invention are characterized in that R_{c}, stands for a human or murine CD40 receptor ligand (CD40L)-derived peptide, with said peptide belonging to the primary sequence of the CD40L ligand of CD40 the amino acid number of which is included between 3 and 10.

According to an advantageous mode of execution, the compounds of the invention are characterized in that R_{c} stands for a human or murine CD40 receptor ligand (CD40L)-derived peptide, with said peptide belonging to the primary sequence of the CD40L ligand the amino acid number of which is included between 3 and 10 selected among the following sequences:

| | |
|---|---|
| LQWAEKGYYTMSNN; | (human sequence SEQ ID NO: 1) |
| LQWAKKGYYTMKSN; | (murine sequence SEQ ID NO: 2) |
| PGRFERILLRAANTH; | (human sequence SEQ ID NO: 3) |
| SIGSERILLKAANTH. | (murine sequence SEQ ID NO: 4) |

The instant invention relates to compounds as specified hereinabove, characterized in that R_{c} stands for a group of formula H—Xₐ—(X_{b})ₗ—X_{c}—X_{d}—Xₑ—(X_{f})ᵢ— or H—X′ₐ-L-X′_{b}—X_{c}—X_{d}—Xₑ—(X_{f})ᵢ—; wherein
l denotes 0 or 1,
i denotes 0 or 1,
Xₐ is selected among the following amino acid residues:
lysine;
arginine;
ornithine;
β-amino acids corresponding to lysine, arginine or ornithine bearing the substitution in positions α or β;
tranexamic acid;
N-methyl-tranexamic acid;
8-amino-3,6-dioxaoctanoic acid;
4(piperidin-4-yl)butanoic acid;
3(piperidin-4-yl)propionic acid;
N-(4-aminobutyl)-glycine;
NH₂—(CH₂)ₙ—COON, n ranging from 1 to 10;
NH₂—(CH₂—CH₂—O)ₘ—CH₂CH₂COOH, with m ranging from 3 to 6;
4-carboxymethyl-piperazine;
4-(4-aminophenyl)butanoic acid;
3-(4-aminophenyl)propanoic acid;
4-aminophenylacetic acid;
4-(2-aminoethyl)-1-carboxymethyl-piperazine;
trans-4-aminocyclohexanecarboxylic acid;
cis-4-aminocyclohexanecarboxylic acid;
cis-4-aminocyclohexane acetic acid;
trans-4-aminocyclohexane acetic acid;
4-amino-1-carboxymethyl piperidine;
4-aminobenzoic acid;
4(2-aminoethoxy)benzoic acid;
X_{b} is selected among the following amino acid residues:
glycine;
isoleucine;
leucine;
valine;
asparagine;
D-alanine;
D-valine;
L-proline optionally substituted in position β, γ or δ;
D-proline optionally substituted in position β, γ or δ;
N-alkyl-natural amino acids, the alkyl group being a methyl, ethyl or benzyl group;
dialkyl-acyclic amino acids of the following formula:

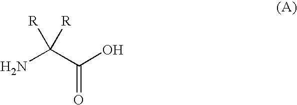

R designating H, Me, Et, Pr or Bu;
dialkyl-cyclic amino acids of the following formula:

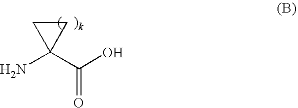

k designating 1, 2, 3 or 4;
X_{c} is selected among the following amino acid residues:
tyrosine;
isoleucine;
leucine;
valine;

phenylalanine;
3-nitro-tyrosine;
4-hydroxymethyl-phenylalanine;
3,5-dihydroxy-phenylalanine;
2,6-dimethyl-tyrosine;
3,4-dihydroxy-phenylalanine (DOPA);
X_{d} is selected among the following amino acid residues:
tyrosine;
isoleucine;
leucine;
valine;
phenylalanine;
3-nitro-tyrosine;
4-hydroxymethyl-phenylalanine;
3,5-dihydroxy-phenylalanine;
2,6-dimethyl-tyrosine;
3,4-dihydroxy-phenylalanine;
Xₑ is selected among the following amino acid residues:
arginine;
-(Gly)ₙ-, n ranging from 1 to 10;
-(Pro)ₙ-, n ranging from 1 to 10;
NH₂—(CH₂)ₙ—COON, n ranging from 1 to 10;
NH₂—(CH₂—CH₂—O)ₘ—CH₂CH₂COOH, m ranging from 3 to 6;
8-amino-3,6-dioxaoctanoic acid;
tranexamic acid;
N-methyl-tranexamic acid;
4(piperidin-4-yl)butanoic acid;
3(piperidin-4-yl)propionic acid;
N-(4-aminobutyl)-glycine;
4-carboxymethyl-piperazine;
4-(4-aminophenyl)butanoic acid;
3-(4-aminophenyl)propanoic acid;
4-aminophenylacetic acid;
4-(2-aminoethyl)-1-carboxymethyl-piperazine;
trans-4-aminocyclohexanecarboxylic acid;
cis-4-aminocyclohexanecarboxylic acid;
cis-4-aminocyclohexane acetic acid;
trans-4-aminocyclohexane acetic acid;
4-amino-1-carboxymethyl piperidine;
4-aminobenzoic acid;
4(2-aminoethoxy)benzoic acid;
X_{f} is selected among the following amino acid residues:
-(Gly)ₙ-, n ranging from 1 to 10;
-(Pro)ₙ-, n ranging from 1 to 10;
NH₂—(CH₂)ₙ—COON, n ranging from 1 to 10;
NH₂—(CH₂—CH₂—O)ₘ—CH₂CH₂COOH, m ranging from 3 to 6;
8-amino-3,6-dioxaoctanoic acid;
tranexamic acid;
N-methyl-tranexamic acid;
4(piperidin-4-yl)butanoic acid;
3(piperidin-4-yl)propionic acid;
N-(4-aminobutyl)-glycine;
4-carboxymethyl-piperazine;
4-(4-aminophenyl)butanoic acid;
3-(4-aminophenyl)propanoic acid;
4-aminophenylacetic acid;
4-(2-aminoethyl)-1-carboxymethyl-piperazine;
trans-4-aminocyclohexanecarboxylic acid;
cis-4-aminocyclohexanecarboxylic acid;
cis-4-aminocyclohexane acetic acid;
trans-4-aminocyclohexane acetic acid;
4-amino-1-carboxymethyl piperidine;
4-aminobenzoic acid;
4(2-aminoethoxy)benzoic acid;
-L- denotes a peptide-like type bond between residues X′ₐ and X′_{b} selected in particular from the list given below:

-L-=-ψ(CH₂CH₂)—; -ψ(CH(Fₖ)═CH(Fₖ′))-; -ψ(CH₂NH)—; -ψ(NHCO)—;

-ψ(NHCONH)—; -ψ(CO—O)—; -ψ(CS—NH)—; -ψ(CH(OH)—CH(OH))—; -ψ(S—CH₂)—;

-ψ(CH₂—S)—; -ψ(CH(CN)—CH₂)—; -ψ(CH(OH))—; -ψ(COCH₂)—; -ψ(CH(OH)CH₂)—;

-ψ(CH(OH)CH₂NH)—; -ψ(CH₂)—; -ψ(CH(Fₖ))—; -ψ(CH₂O)—; -ψ(CH₂—NHCONH)—; -ψ(CH(Fₖ)NHCONFₖ′)-; -ψ(CH₂—CONH)—;

-ψ(CH(Fₖ)CONH)—; -ψ(CH(Fₖ)CH(Fₖ′)CONH)—;

or -ψ(CH₂N)—; -ψ(NHCON)—; -ψ(CS—N)—; -ψ(CH(OH)CH₂N)—;

-ψ(CH₂—NHCON)—; -ψ(CH₂—CON)—; -ψ(CH(Fₖ)CON)—;

-ψ(CH(Fₖ)CH(Fₖ′)CON)—;

if X′_{b} stands for a proline side chain,

Fₖ and Fₖ' denote, independently from each other, a hydrogen, a halogen, an alkyl group of 1 to 20 carbon atoms, or an aryl group the ring structure of which includes from 5 to 20 carbon atoms,
X′ₐ designates the side chain of lysine, arginine or ornithine; and
X′_{b} designates the side chain of one of the following amino acid residues:
glycine;
isoleucine;
leucine;
valine;
asparagine;
D-alanine;
D-valine;
L-proline optionally substituted in position β, γ or δ;
D-proline optionally substituted in position β, γ or δ;
N-alkyl natural amino acids, the alkyl group of which is a methyl, ethyl or benzyl group;
dialkyl-acyclic amino acids of the following formula:

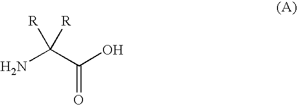

R designating H, Me, Et, Pr or Bu;
dialkyl-cyclic amino acids of the following formula:

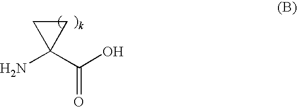

k designating 1, 2, 3 or 4.

Hence, Xₐ is chosen in particular among the following groups:

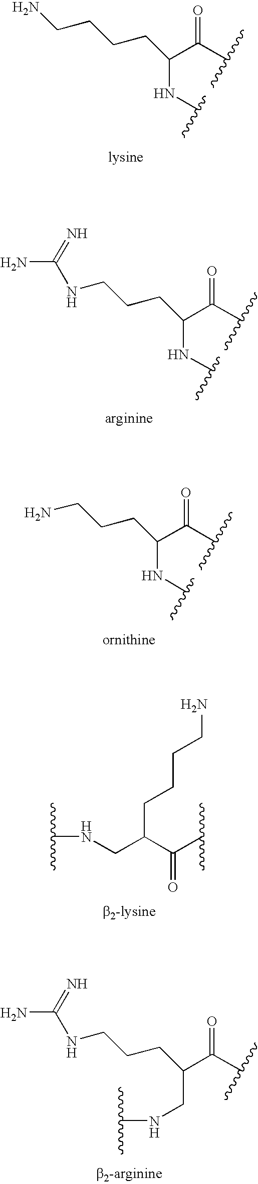

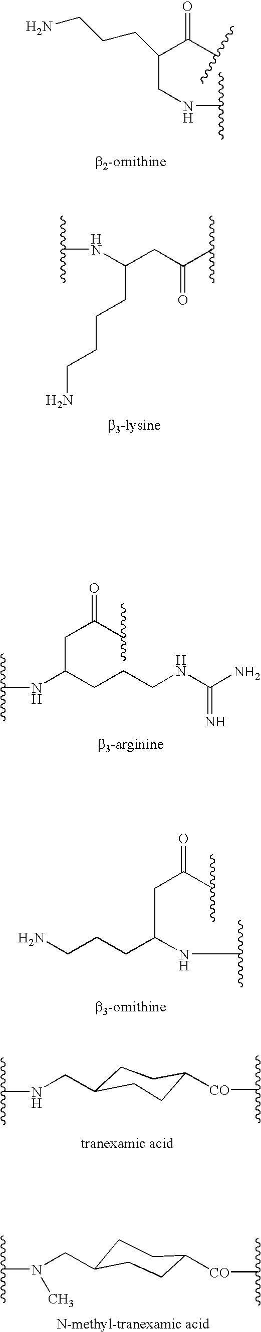

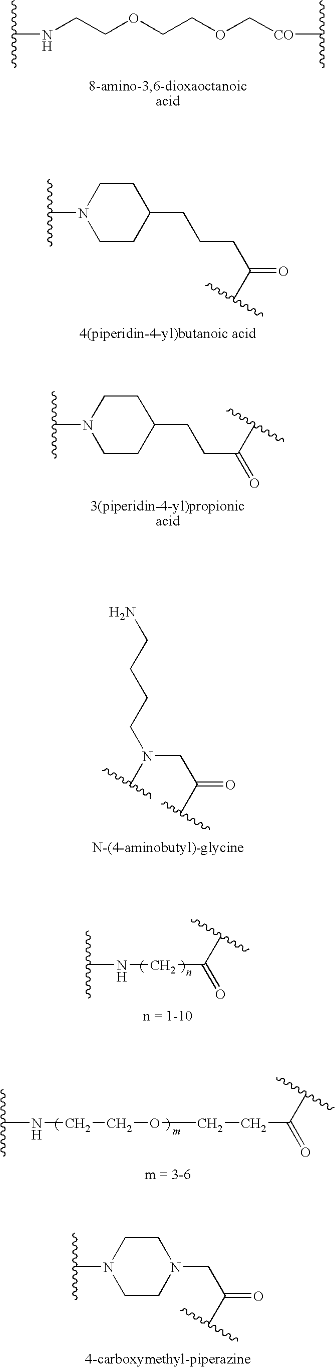

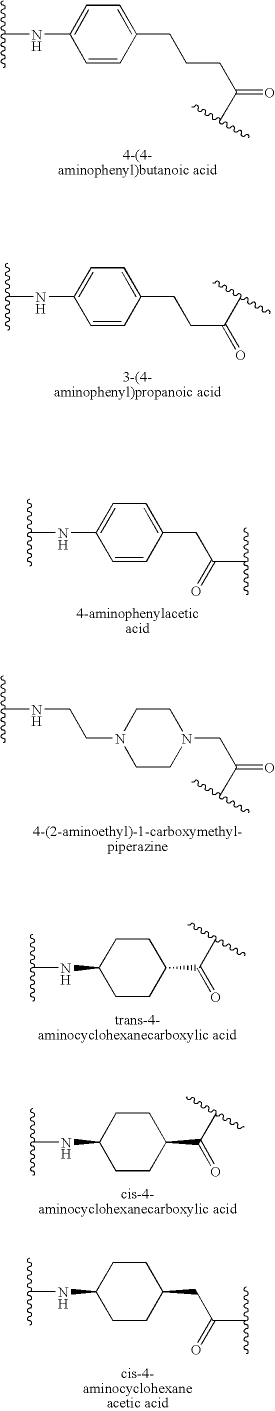

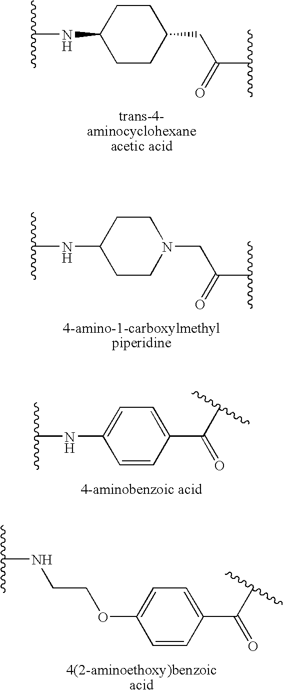

X_{b} is chosen in particular among the following groups:

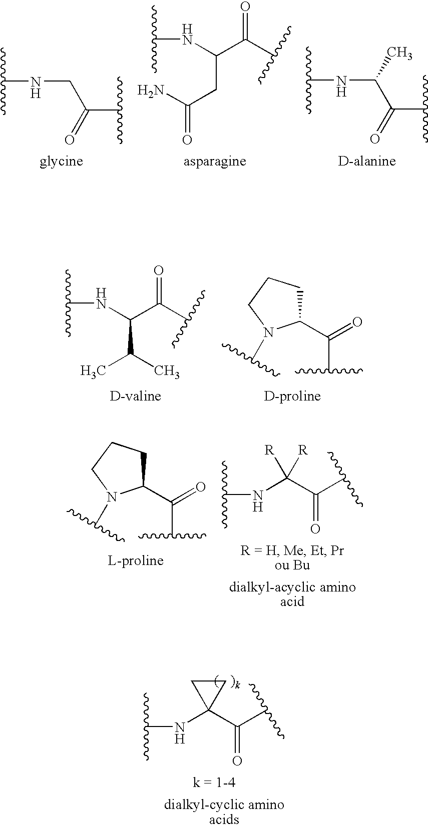

X_{c} and X_{d} are chosen in particular among the following groups:

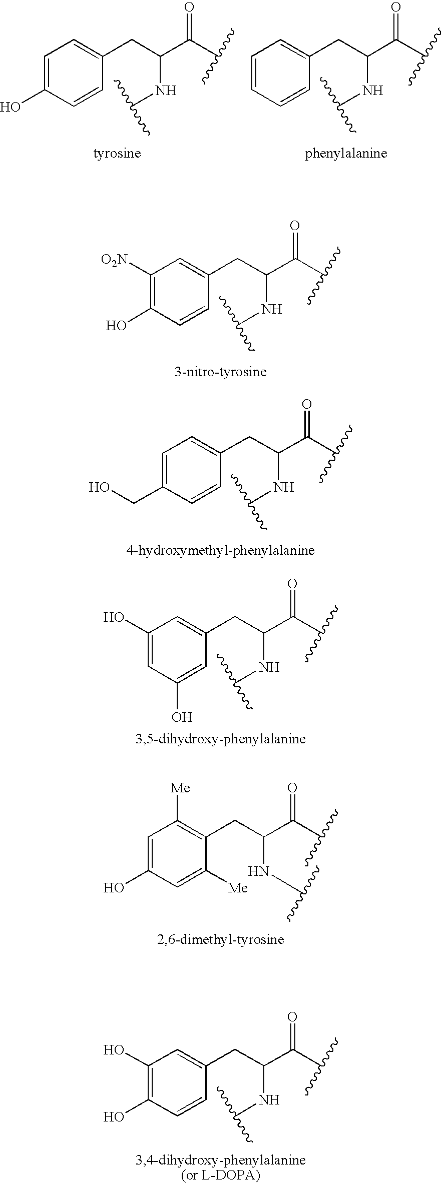

Xₑ and X_{f} are chosen in particular among the following groups:

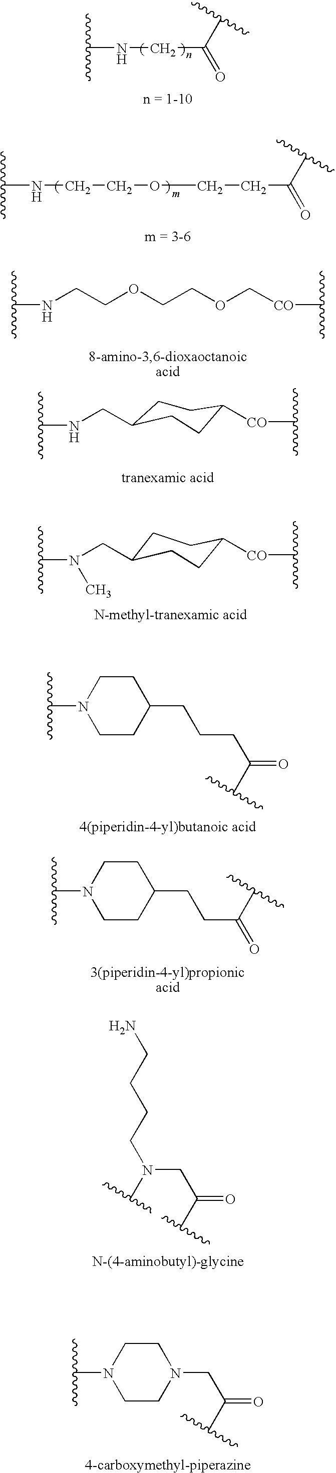

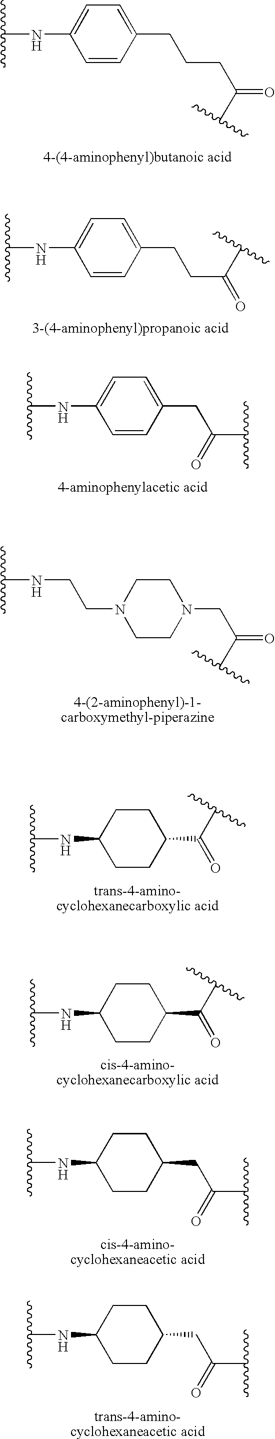

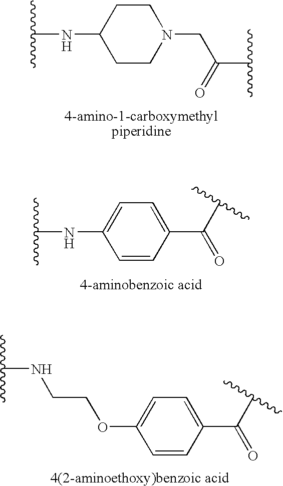

The instant invention also relates to compounds of formula (I) as specified hereinabove, characterized in that Y has the following formula (II):

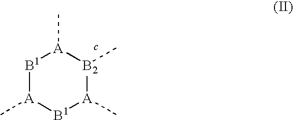

wherein:
A designates an amino acid residue or an amino acid derivative randomly selected among:

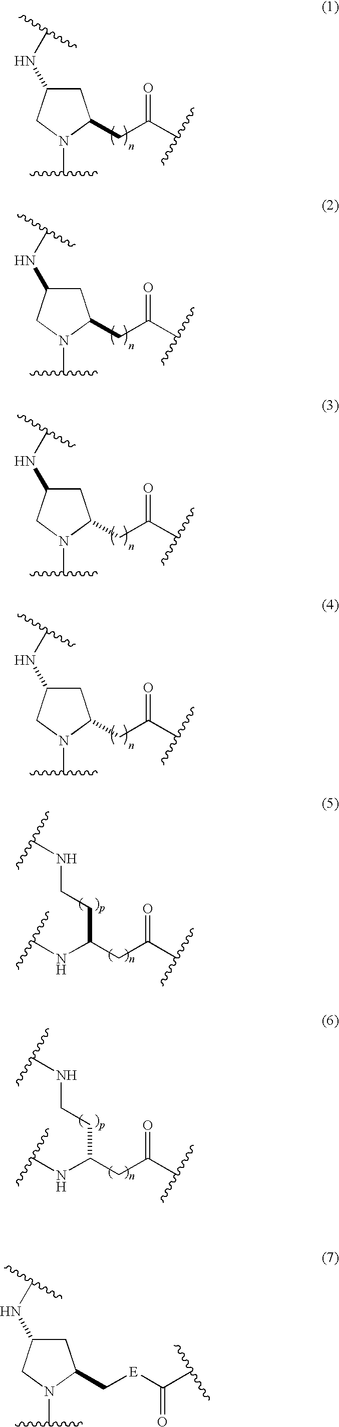

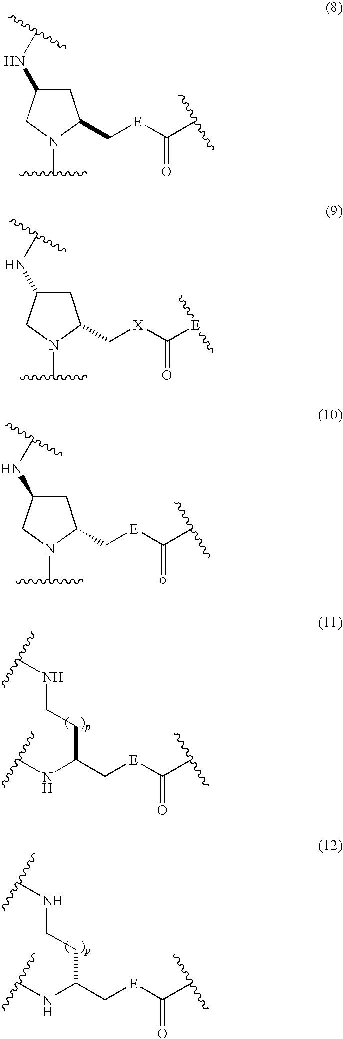

n designating 0, 1, 2 or 3;
p designating 0, 1, 2 or 3;
E designating NH or 0;
B¹ designates an amino acid residue or an amino acid derivative independently selected among:

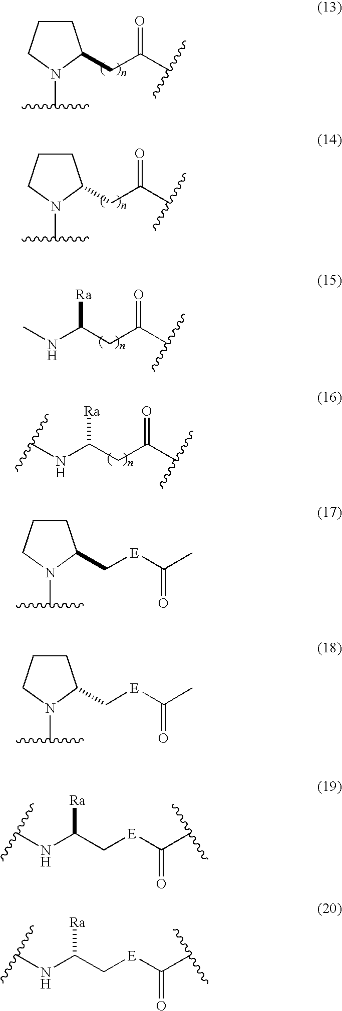

n being equal to 0, 1, 2 or 3;
p being equal to 0, 1, 2 or 3;
E designating NH or O;
   Rₐ stands for the carbon chain of a C1-C8 alkylated proteinogenic amino acid,
B² is independently selected among the following groups: the c bond designating the bond to the X group,

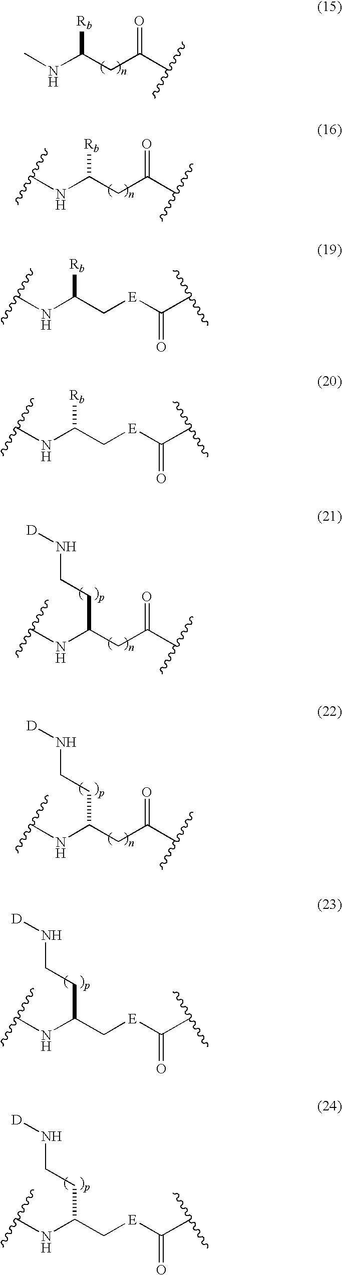

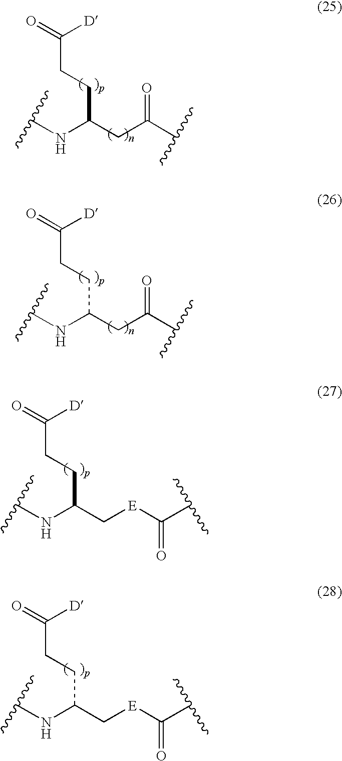

n being equal to 0, 1, 2 or 3;
p being equal to 0, 1, 2 or 3;
E designating NH or O ;
   R_{b} stands for an alkyl chain including from 1 to 6 carbon atoms;
   D′ designating one of the following groups:

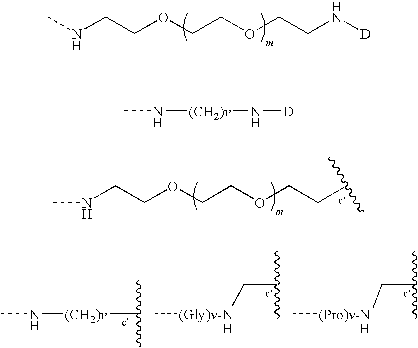

m being an integer ranging from 1 to 40;
v being an integer ranging from 1 to 10;
the bond c′ designating the bond to the X group,
D designating the following groups:
   a group of formula

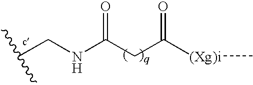

where X stands for a group of formulae (13′) and (15′)
or a group of formula

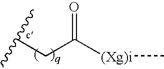

or of formula

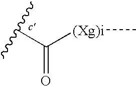

where X stands for a group of formulae (1′), (2′), (8′), (9′), (5′), (6′), (7′), (10′), (11′), (12′) and (14′),
the c′ bond designating the bond to the X group,
q designating an integer ranging from 2 to 6;
X_{g} corresponding to a residue of one of the following groups:
   -(Gly)ₙ-, n ranging from 1 to 10;
   -(Pro)ₙ-, n ranging from 1 to 10;
   NH₂—(CH₂)ₙ—COON, n ranging from 1 to 10;
   NH₂—(CH₂—CH₂—O)ₘ—CH₂CH₂COOH, m ranging from 3 to 6;
   8-amino-3,6-dioxaoctanoic acid;
   tranexamic acid;
   N-methyl-tranexamic acid;
   4(piperidin-4-yl)butanoic acid;
   3(piperidin-4-yl)propionic acid;
   N-(4-aminobutyl)-glycine;
   4-carboxymethyl-piperazine;
   4-(4-aminophenyl)butanoic acid;
   3-(4-aminophenyl)propanoic acid;
   4-aminophenylacetic acid;
   4-(2-aminoethyl)-1-carboxymethyl-piperazine;
   trans-4-aminocyclohexanecarboxylic acid;
   cis-4-aminocyclohexanecarboxylic acid;
   cis-4-aminocyclohexane acetic acid;
   trans-4-aminocyclohexane acetic acid;
   4-amino-1-carboxymethyl piperidine;
   4-aminobenzoic acid;
   4(2-aminoethoxy)benzoic acid.

In accordance with an advantageous embodiment, the compounds of the invention are compounds of formula (I) as specified above wherein Y has one of the following formulae:

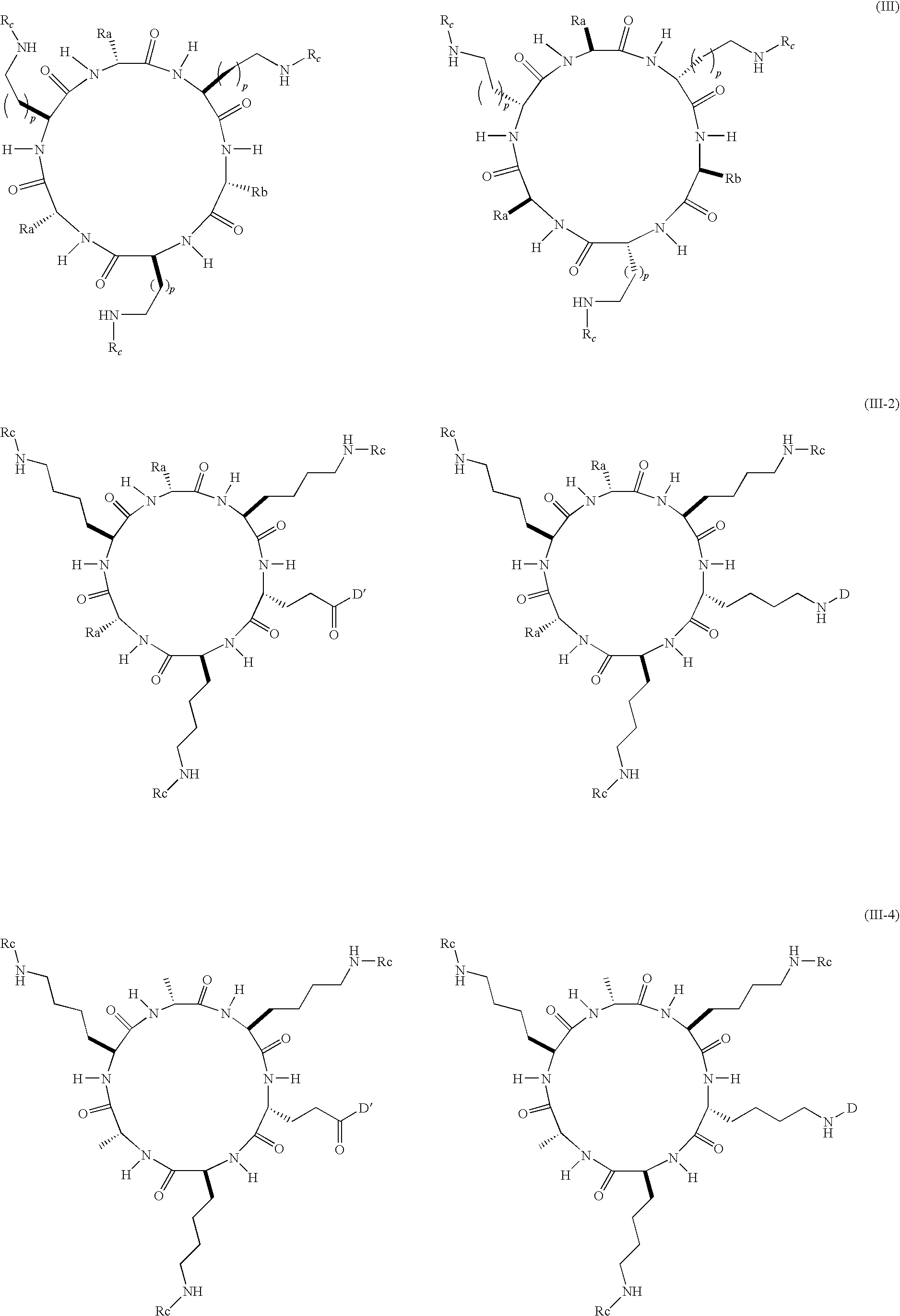

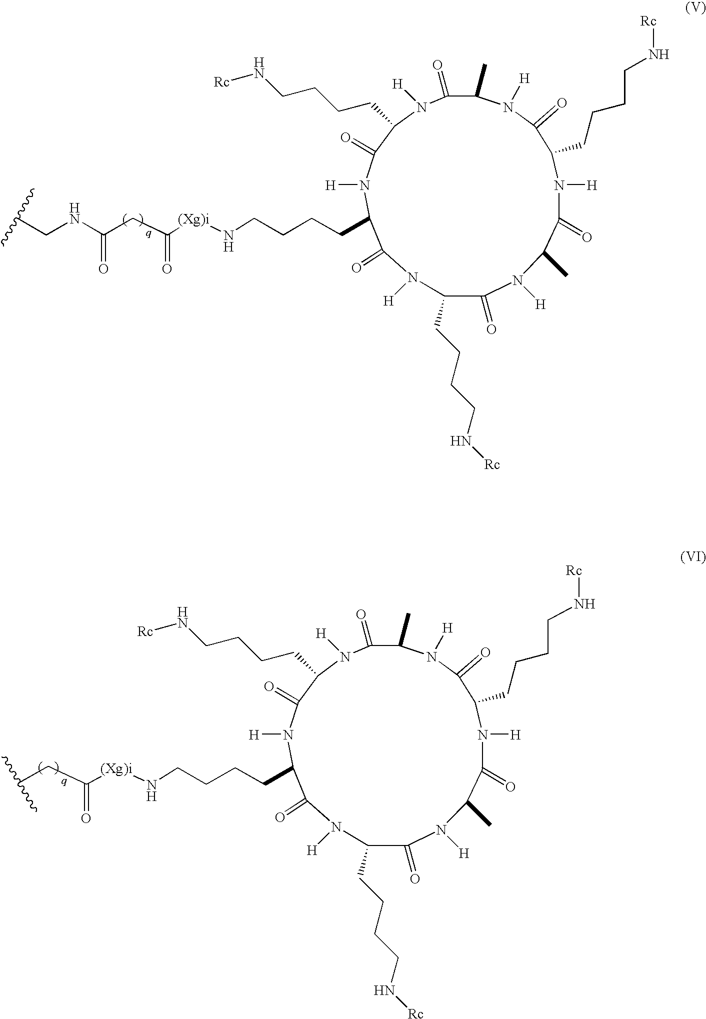

Rₐ, R_{c}, D, D′, X_{g}, q, i and p being as specified above.

Preferred compounds in accordance with the invention are compounds having one of the following formulae (III-a) or (III-b):

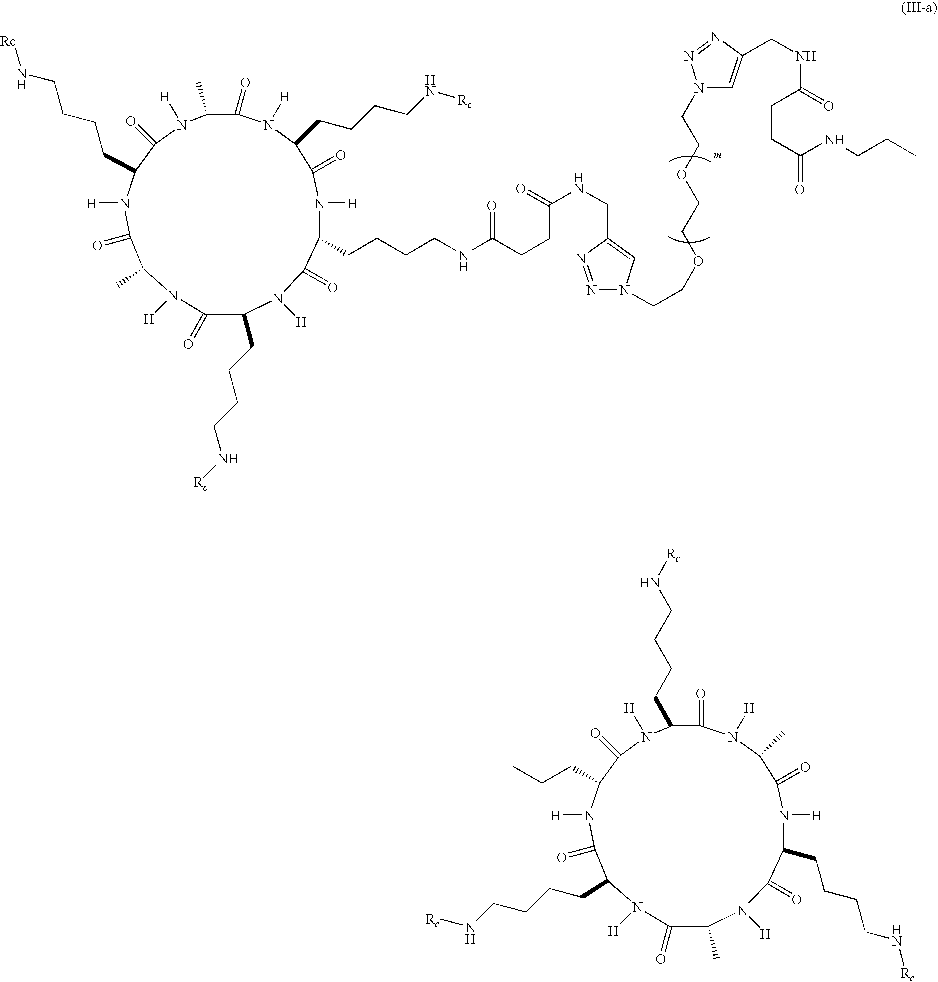

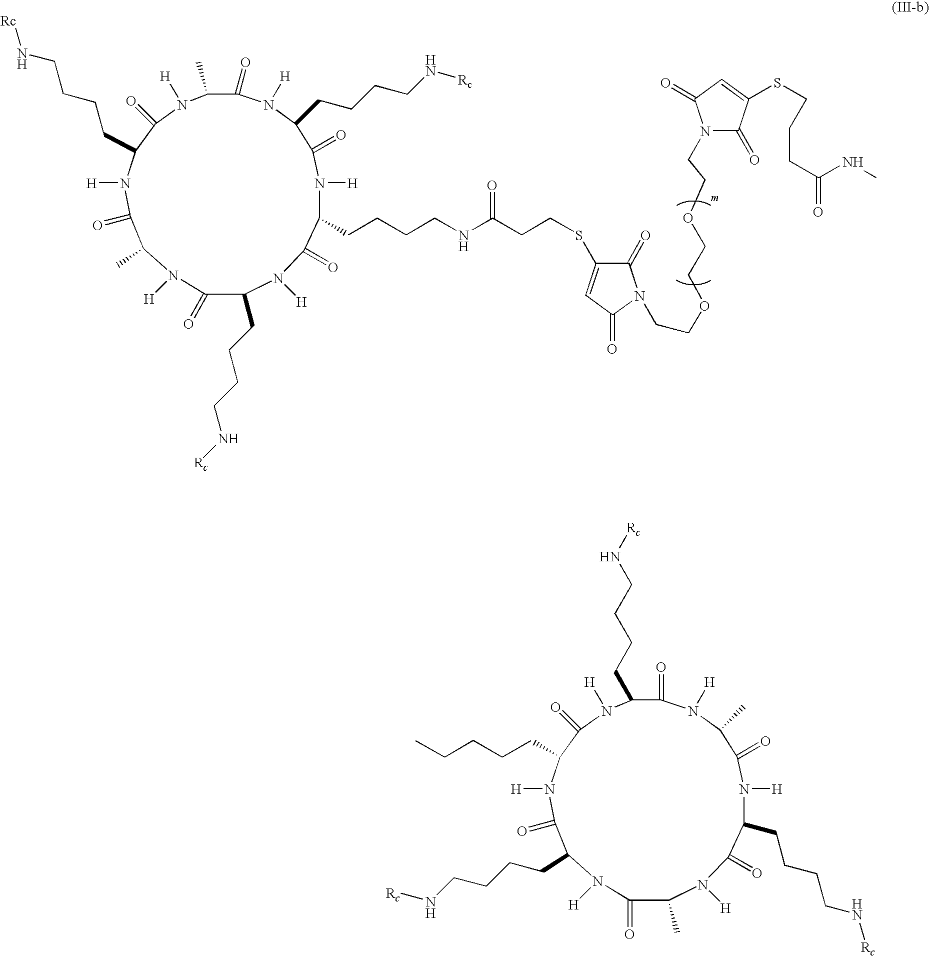

R_{c} and m being as specified above.

According to an advantageous embodiment, the compounds of the invention are characterized in that R_{c} is selected among one of the following groups:
H-Lys-Gly-Tyr-Tyr-NH—(CH₂)₅—CO—
H-Lys-(D)Pro-Tyr-Tyr-NH—(CH₂)₅—CO—
Ahx-(D)Pro-Tyr-Tyr-NH—(CH₂)₅—CO—
H-Lys-ψ(CH₂N)-(D)Pro-Tyr-Tyr-NH—(CH₂)₅—CO—, -ψ(CH₂N)— corresponding to a peptide-like bond of a methylene-amino type,
H-Lys-ψ(CH₂)-Gly-Tyr-Tyr-NH—(CH₂)₅CO—, -ψ(CH₂)— corresponding to a peptide-like bond of a methylene type,
H-Lys-ψ(CH₂—CH₂)-Gly-Tyr-Tyr-NH—(CH₂)₅CO—, -ψ(CH₂CH₂ corresponding to a peptide-like bond of an ethylene type,
H-Lys-Gly-DOPA-Tyr-NH—(CH₂)₅—CO—, or
H-Arg-Ile-Ile-Leu-Arg-NH—(CH₂)₅—CO—.

The present invention is further directed to a compound as specified above of the following formula (III-a):

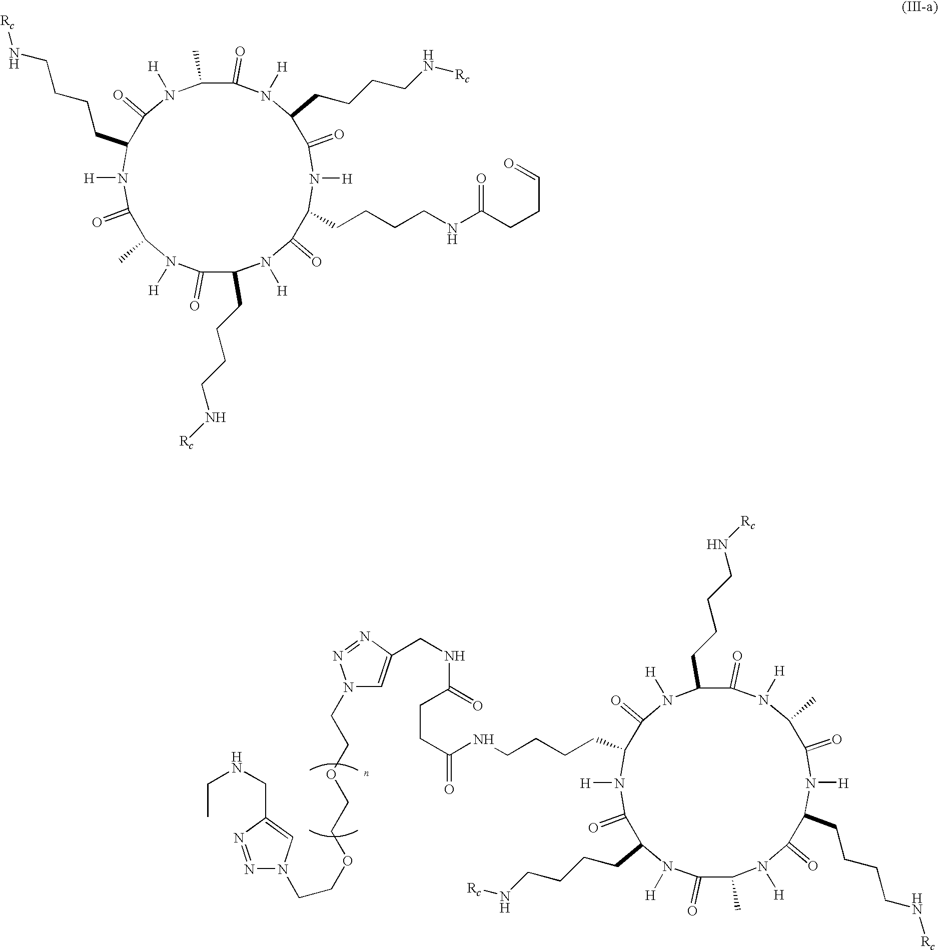

wherein:
n is equal to 1, 2 or 6;
R_{c} stands for a H-Lys-Gly-Tyr-Tyr-NH—(CH₂)₅—CO— group.

The present invention also deals with a pharmaceutical composition is characterized in that it comprises, as an active ingredient, a compound of formula (I) as specified hereinabove, in combination with a pharmaceutically acceptable carrier .

The present invention also deals with a vaccine composition, characterized in that it includes, as an active ingredient, a compound of formula (I) as specified hereinabove, in combination with a pharmaceutically acceptable adjuvant.

The present invention is further directed to the use of compounds as specified above, for preparing a medicinal drug intended for the treatment of pathologies involving the inhibition or activation of the immune response.

The immune response should be inhibited in the course of inflammatory disease (inflammatory rhumatoid affections), autoimmune diseases, hypersensitivity reactions in general and allergic disorders in particular, graft rejection, graft versus host reactions.

The immune response should be activated in general vaccination procedures, in cancer immunotherapy, in bacterial or viral infections inducing an immunodepression (measles, AIDS, herpes virus, cytomegalovirus . . . ), in treating bacterial or viral infections or disease involving non conventional infectious agents (prions) in subjects suffering from primary or secondary immunodeficiency.

The present invention is further directed to the use of compounds as specified above, for preparing a medicinal drug intended for treating pathologies involving inhibition of the immune reponse, such as graft rejection, allergic disorders and autoimmune diseases.

The present invention is further directed to the use as specified above, for preparing a medicinal drug intended for treating pathologies involving an increase of the immune response, such as cancers or parasitic, bacterial or viral infections or else involving non conventional infectious agents such as prions.

Diseases involving the inhibition of the immune response include autoimmune diseases such as diabetes, multiple sclerosis, disseminated lupus erythrormatosis or rhumatoid arthritis, graft rejection, especially in dealing with allografts, xenografts or graft versus host reactions, as well as hypersensitivity reactions such as allergic conditions, especially hay fever and atopic dermatitis or granulomas.

Compounds in accordance with the present invention, used in inhibiting the immune response, can be administered by intravenous, intramucosal (by oral, intranasal, intravaginal means, by inhalation), subcutaneous, intradermal or epidermal routes.

The present invention also applies to a pharmaceutical composition, characterized in that it includes a compound according to the invention, for treating pathologies involving the inhibition of the immune response, which compound is present in the pharmaceutical composition in such quantities as to be administered at a rate of about 100 ng to about 5 mg by subject daily.

The present invention also pertains to the use as mentioned above, for preparing a medicinal drug intended for the treatment of pathologies involving an increase of the immune response, such as cancers or parasitic, bacterial or viral infections or else involving non conventional infectious agents such as prions.

Cases involving activation of the immune response include vaccination procedures in general, especially vaccines against influenza or against paediatric diseases, cancer immunotherapy, especially in the management of melanomas or metastatic cancer, or bacterial or viral disease inducing immunosuppression in particular in the management of measles, AIDS, herpes virus or cytomegalovirus, or vaccines intended for subjects suffering from a primary or secondary immunodeficiency.

Compounds according to the present invention, used in activating the immune response, can be administered by intraveous, intramucosal (by oral, intranasal, intravaginal means, by inhalation), subcutaneous, intradermal or epidermal routes.

The present invention is also concerned with a pharmaceutical composition, characterized in that it includes a compound according to the present invention, for treating pathologies involving an activation of the immune response, which compound is present in the pharmaceutical composition in such quantities as to be administered at a rate of about 100 ng to about 5 mg by subject daily.

The present invention also deals with a process for preparing on a solid support a compound of formula (I), as specified above, with said process being is characterized in that it comprises the following steps:
forming a linear precursor of Y as specified above, which precursor is constituted of an amino acid sequence forming a growing peptide chain, being synthesized by successive coupling cycles of N-protected amino acid residues, three of which bear an amine type group, and the amine functionality of the growing peptide chain, and deprotecting, the first amino acid residue being bound to a solid support, said linear precursor of Y comprising at least one D-alanine residue, said D-alanine residue being substituted by a D-lysine residue the ε-NH amine of which has been acylated by a carboxylic acid bearing the desired functionality corresponding to an X group as specified above,
cyclizing the linear precursor of Y in a protected state mentioned above, in order to obtain a protected functionally substituted ring,
reacting said protected functionally substituted ring with a Z-derived spacer as specified above, in order to obtain a dimerized protected functionally substituted ring,
cleaving said previously mentioned protecting groups, to free said previously mentioned protected amine functionalities and obtain a dimerized deprotected functionally substituted ring,
coupling said previously mentioned freed amine functionalities with an in situ formed or preformed peptide by successive assembly of amino acid residues corresponding to the R_{c} group as specified above, and
cleaving the molecule from the solid support, after removal of all protecting groups present on functionally substituted side chains of the R_{c} group, in order to obtain the compound as specified above.

The present invention also deals with a process as specified above for preparing a compound of formula (III-a) or (III-b), said process being characterized in that it includes the following steps:
reacting a protected functionally substituted ring of the following formula:

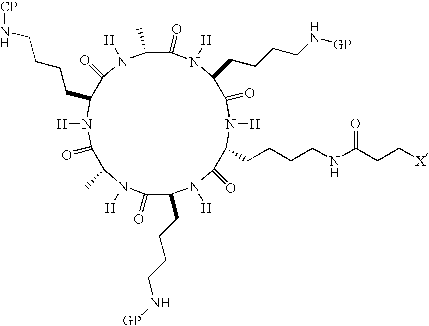

GP denoting a protecting group chosen in particular among: Boc, Z or Alloc,

X′ denoting a —SH group or

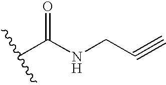

with a spacer group of formula

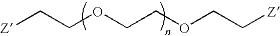

n denoting an integer ranging from 1 to 10

Z denoting a N₃ group or a group

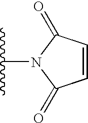

it being understood that when X′ denotes a group

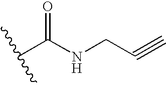

Z′ denotes a N₃ group,

and when X′ denotes a —SH group, Z′ denotes a group

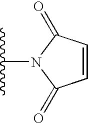

in order to obtain a compound of the following formula (VIII):

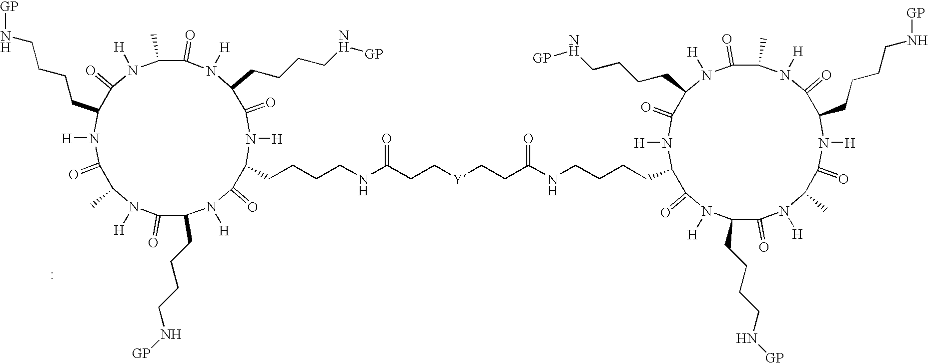

GP being as specified above,

Y′ having one of the formulae given below:

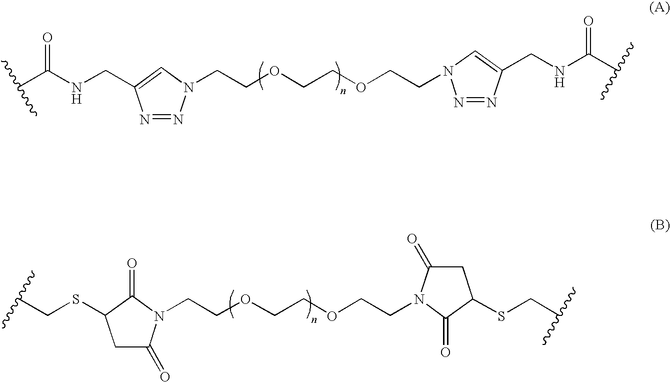

it being understood that Y′ has formula (A) if Z′ denotes a N₃ group

and that Y′ has formula (B) if Z′ denotes a group

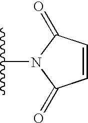

deprotecting said previously mentioned protecting groups GP, to free protected amine functionalities and coupling said previously mentioned freed amine functionalities to an in situ formed or preformed peptide by successive assembly of corresponding amino acid residues to the R_{c} group as specified above, and
cleaving the molecule from its solid support, following removal of all protecting groups present on functionally substituted side chains of the R_{c} group, in order to obtain the compound of formula (III-a) or (IIIb) as specified above.

### DESCRIPTION OF THE FIGURES

FIG. 1 shows the synergic effect between L1 (see formula hereinafter) and the anti-CD40 antibody agonistic agent 3/23. Uptake of ³H-thymidine in cpm is shown on the ordinate axis.

FIG. 2 shows the HPLC elution profile of the 1,3-diplolar reaction between the macrocycle IV.23 and the IV.15 diazide under conditions specified by entry 6 and following treatment with TFA (HPLC: linear gradient, 5±65% B, 20 min.)

FIG. 3 shows the HPLC elution profile of 1,3-dipolar reaction between the macrocycle IV.23 and the IV.14 diazide under conditions specified by entry 5 and following treatment with TFA (HPLC: linear gradient, 5±65% B, 20 min.)

FIG. 4A shows the HPLC elution profile of the crude coupling reaction medium between the dimerized macrocycle IV.25 and the protected pentapeptide P1 following treatment with TFA. FIG. 4B shows the HPLC elution profile of the L41 ligand following purification by preparative HPLC (HPLC: linear gradient, 5-65% B, 20 min.). FIG. 4C shows the MALDI-TOF spectrum profile of the L41 ligand (expected mass: 5529).

FIGS. 5A and 5B show apoptosis induction on Burkitt's lymphoma RAJI cells and B cell proliferation induction by L1 and L41. The white bars stand for the L41 ligand while the black bars stand for the L1 ligand.

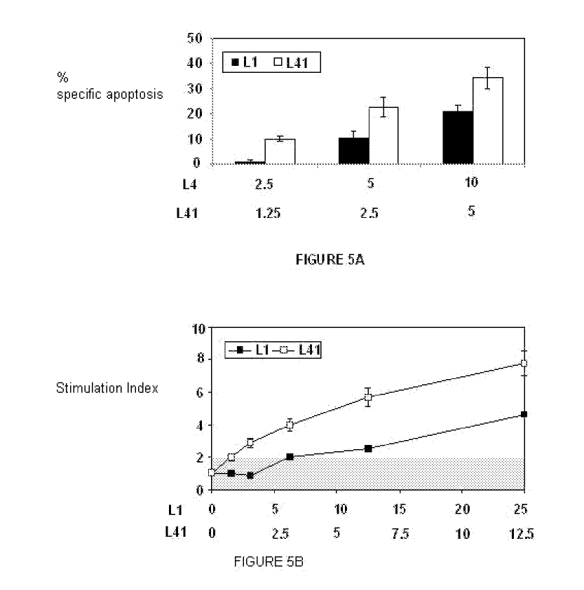

In FIG. 5A, the percentage of specific apoptosis is plotted on the ordinate axis while the ligand concentration in μM is plotted on the abscissa axis. Results stand for the arithmatic mean of three independent experiments

+/−SD.

In FIG. 5B, the ordinate axis shows the stimulation index corresponding to cpm values obtained in cultures with CD40L analogs divided by cpm values obtained in cultures without CD40L analogs, whereas the abscissa axis shows the concentration of each analog of CD40L (L1 and L41). The white squares stand for the L41 ligand while the black squares stand for the L1 ligand.

By interacting with their respective ligands, the receptors of the TNF superfamily transmit a signal which enables cell survival, proliferation and differentiation and/or apoptosis (programmes cell death). Based on the particular receptor of the TNF superfamily being considered, and/or cellular type, the extent of oligomerization required for signal transduction differs: some are activated by the homotrimeric ligand in soluble form whereas others are only activated by the membrane bound homotrimeric ligand. Thus, the TWEAK, TNF and BAFF ligands are active in the soluble form. By contrast, activation by FasL, TRAIL, CD40L or CD30L requires one or more spatially close homotrimers to be recruited (Holler, N.; Tardivel, A.; Kovacsovics-Bankowski, M.; Hertig, S.; Gaide, O.; Martinon, F.; Tinel, A.; Deperthes, D.; Calderara, S.; Schulthess, T.; Engel, J.; Schneider, P.; Tschopp, J. Molec. Cell. Biol. 2003, 23, 1428-1440; Schneider, P.; Holler N.; Bodmer, J. L.; Hahne, M.; Frei, M.; Fontana, A.; Tschopp, J. J. Exp. Med. 1998, 187, 1205-1213).

In case of CD40 signalling, it has been demonstrated that for certain cellular types and especially B cells, the formation of a hexameric complex 3:3 alone was not enough to induce signal transduction. In this case, the minimal signalling unit or motif amounts to not one but several hexameric complexes which are close to each other.

This observed fact has driven biochemists to develop tools to increase receptor oligomerization induced by the soluble ligand. This approach makes it possible on one hand, to increase the biological effect triggered by receptor engagement, and on the other, to study mechanisms by which multimerization brings about such an increase.

So far, only biochemical approaches have been described to amplify oligomerization of CD40. Basically, two strategies have been reported. The first one is based on using two partners: the ligand and an oligomerization amplifying agent. The second relies on constructing multimeric fusion protein. Both approaches have been used to amplify the biological effect of CD40L and FasL.

When considering the first strategy, the amplifying agent may be an anti-receptor monoclonal antibody (e.g. an anti-CD40 mAb) acting synergically with the soluble ligand (Pound, J. D.; Challa, A.; Holder, M. J.; Armitage, R. J.; Dower, S. K.; Fanslow, W. C.; Kikutani, H.; Paulie, S.; Gregory, C. D.; Gordon, J. Int. Immunol. 1999, 11, 11-20; Schneider, P.; Holler, N.; Bodmer, J. L.; Hahne, M.; Frei, K.; Fontana, A.; Tschopp, J. J. Exp. Med. 1998, 187, 1205-1213) or else an anti-Flag monoclonal antibody which effects oligomerization of the recombinant ligand fused to a Flag peptide (Haswell, L. E.; Glennie, M. J.; Al-Shamkhani, A. Eur. J. Immunol. 2001, 31, 3094-3100).

The second strategy consists in building a fusion protein comprised of several copies of the ligand. To this end, Jörg Tschopp's team at the University of Lausanne has built a molecule wherein FasL or CD40L is fused to the collagen domain of the ACRP30 protein. This protein, a member of the C1 q superfamily, has a particular 3D configuration and two homotrimeric heads which combine two homotrimeric domains through fusion on a single molecule. In a similar strategy, Hasxell et al. has described a molecule able to present four CD40L homotrimers. This was achieved by replacing the lectin domain of the <<lung surfactant protein-D>> (SP-D: a member of the lectin superfamily) by the C-terminal extracellular domain of CD40L (Haswell, L. E.; Glennie, M. J.; Al-Shamkhani, A. Eur. J. Immunol. 2001, 31, 3094-3100).

The use of these multimeric proteins ACRP30 and SP-D fused to homotrimeric CD40L has demonstrated that the smallest active unit capable of inducing a strong proliferation of mice spleen B lymphocytes is a dimer of homotrimeric CD40L. Combining both strategies namely <<crosslinking>> the fusion protein CD40L: ACRP30 containing a Flag moiety with an anti-Flag antibody significantly increases the effect of CD40L: ACRP30 on B cells, hence demonstrating the need to amplify the degree of receptor oligomerization for effective signalling.

### EXPERIMENTAL SECTION

### A) Chemistry

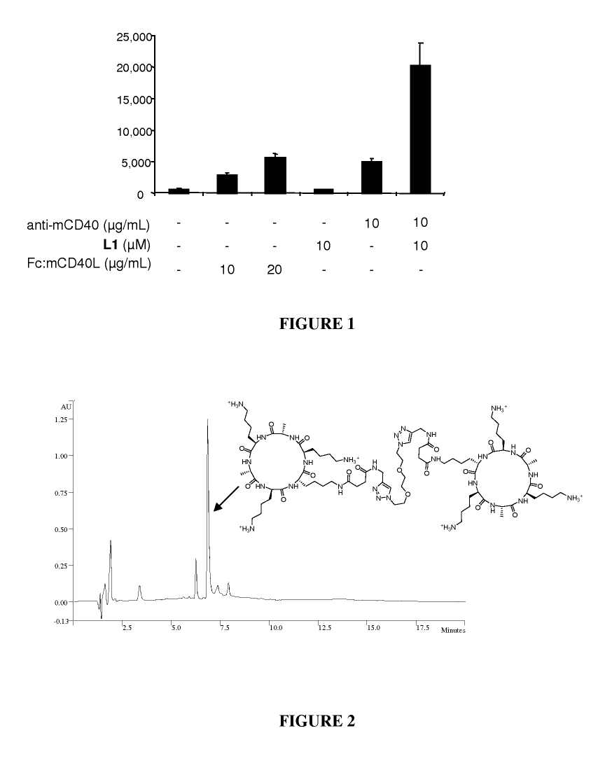

Like the soluble CD40L, synthetic mimetics (e.g. L1 as specified in international application WO03/102207) do not induce the proliferation of murine spleen B cells. Results reported in the literature already discussed here suggest that strategies aimed at further increasing the oligomerization capacity of our ligands are of great relevance. This is corroborated by lab results with the monoclonal antibody 3/23. Indeed, we have successfully shown that the effect of L1 on spleen B cells, in the same way as the effect of soluble homotrimeric CD40L, is boosted by the anti-C40 antibody agonistic agent. Combining these 2 molecules induces B cell proliferation (FIG. 1).

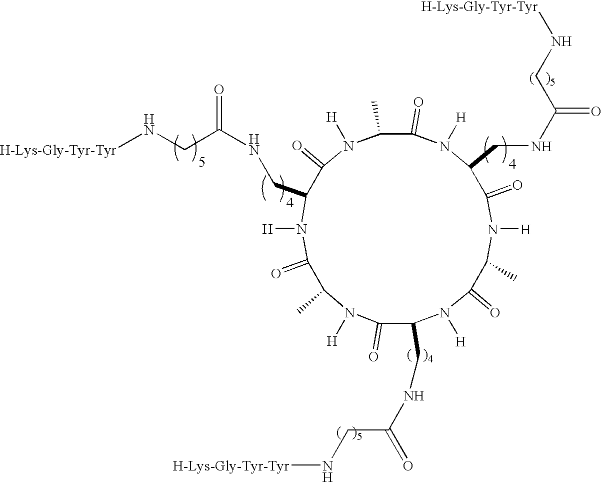

In parallel, the inventors have carried out extensive research and development to design molecules capable of presenting at least two copies of the synthetic ligand simulating homotrimeric CD40L. The underlying concept was to demonstrate an amplifying effect resulting from oligomerization of the ligand and to identify the minimal entity capable of inducing B cell proliferation. To achieve this, one option was to synthesize ligand dimers.

### Synthetic Ligand Dimerization

1) General Retrosynthetic Approach

Dimerized molecules are synthesized starting from two trimeric structures of the cyclo-D, L-α-peptide linked to each other by means of an n-alkyl or polyethylene glycol type spacer. To establish this link, two approaches were contemplated: one uses thiol chemistry (Michael's addition reaction of a thiol to a maleimide) while the other is based on <<click chemistry>>, a dipolar cycloaddition [3+2] reaction (or Huisgen's reaction) between an alkyne and an azide. While in the first approach, the link being introduced is a thioether bond, the <<click chemistry>> approach generates a 1,4-bisubstituted 1,2,3-triazole type bond.

The general strategy used relies on the synthesis of a macrocycle-D, L-α-peptide conjugate to which is attached a functionally substituted group: either a formal alkyne in the case of the <<click chemistry>> approach, or a thiol group in the case of the second approach according to the following scheme:

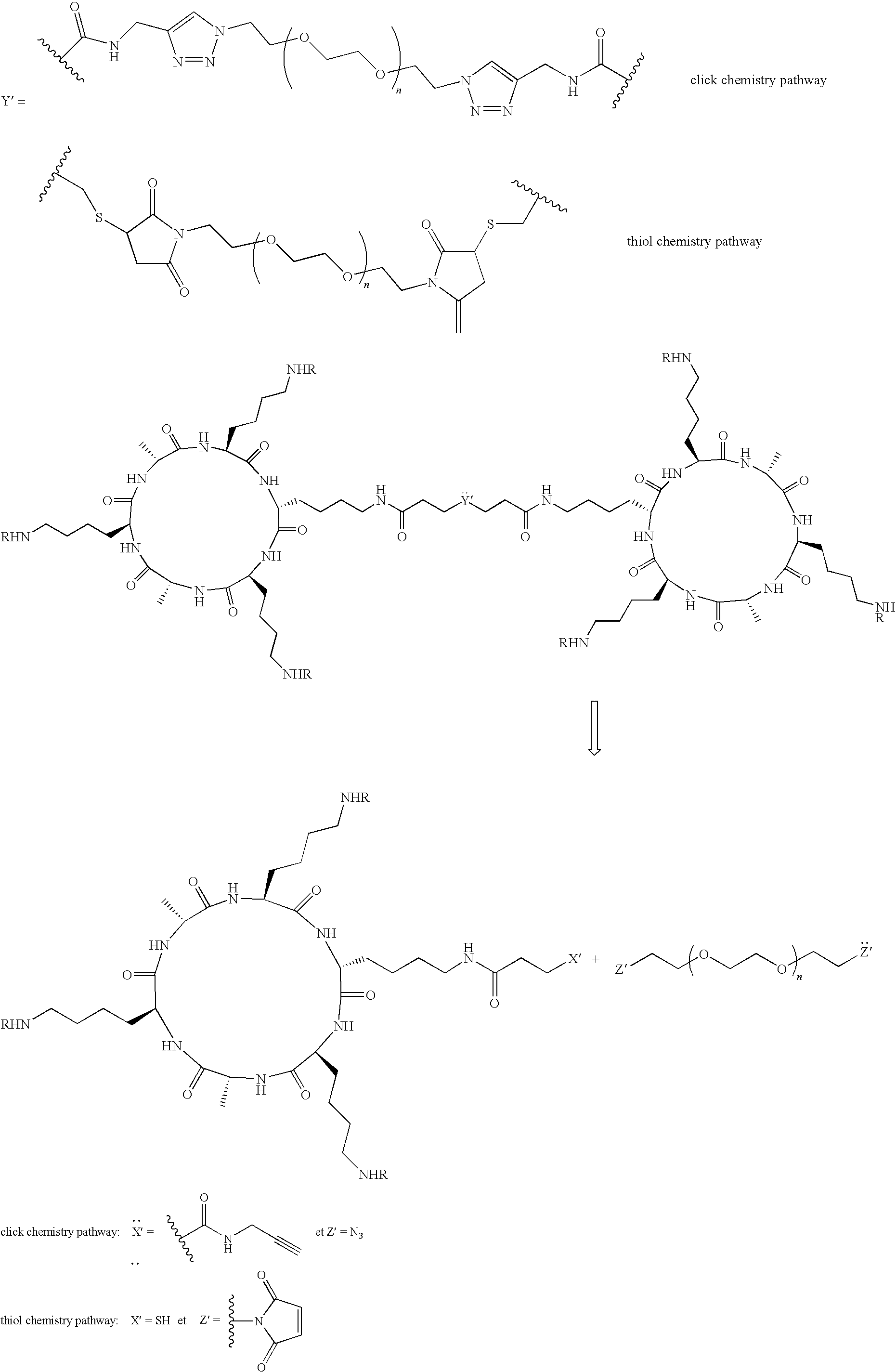

This functionally substituted cycle is prepared according to the following strategy: one of the three D-alanine residues is substituted, in the course of the peptide precursor preparation, by a D-lysine residue the ^{ε}NH amine of which has been acylated beforehand by a carboxylic acid bearing the desired functionality.

2) Dimerization by Michael's Addition Reaction Involving a Thiol and a Maleimide

Initially, it was envisioned to dimerize the functional mimetics of CD40L by Michael's addition reaction between a ligand exocyclicly functionally substituted by a thiol group and a bismaleimide spacer having 6 carbon atoms (IV.1).

This spacer is prepared starting from the corresponding diamine, by reacting the same with N-(Methylcarbonyl)maleimide in a mixture of THF and a saturated solution of sodium hydrogencarbonate ((a) Bodanszky, M.; Bodanszky, A. in the Practice of Peptide Synthesis; Springer-Verlag: New York, 1984, 29-31. (b) Cheronis, J. C.; Whalley, E. T.; Nguyen, K. T.; Eubanks, S. R.; Allen, L. G.; Duggan, M. J.; Loy, S. D.; Bonham, K. A.; Blodgett, K. J. Med. Chem. 1992, 35, 1563-1572). The desired product is obtained by mere washing with no further purification.

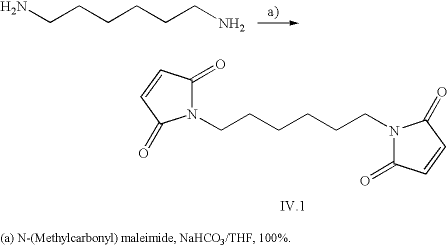

a. Synthesis of the Functionally Substituted Macrocycle

One of the key steps of this synthesis pathway is the preparation of a macrocycle molecule functionally substituted by a thiol group. In a first stage, is there was a need to find a convenient protecting group for this functionality, orthogonal to the Boc group but being stable in typical peptide synthesis conditions. The acetamide group (Acm) was used for this purpose. In fact, this protecting group is stable in Fmoc group deprotection conditions (20% piperidine in DMF) but also in the presence of trifluoroacetic acid.

The synthesis of the intermediate product 3-(Acetylamino-methylsulphanyl) propionic acid (IV.2) required for functionally substituting the macrocycle moiety is outlined in the following scheme:

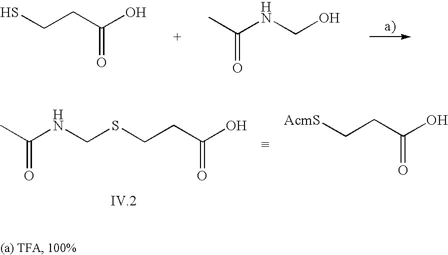

The protection of the 3-mercaptopropanoic acid thiol derivative by the acetamide protecting group is performed in trifluoroacetic acid in the presence of N-(hydroxymethyl)acetamide (Phelan, J. C.; Skelton, N. J.; Braisted, A. C.; McDowell, R. S. J. Am. Chem. Soc. 1997, 119, 455-460).

Acid IV.2 is then coupled to the ^{ε}NH amine of the adequately protected IV.4 D-lysine derivative. The IV.5 compound thus obtained is subsequently deprotected by removing such Fmoc and benzyl ester groups. Next, the N-terminal portion of the zwitterion thus formed is again protected by a Fmoc group in order to obtain the IV.7 precursor required for assembling a straight chain peptide intended for subsequent macrocyclization.

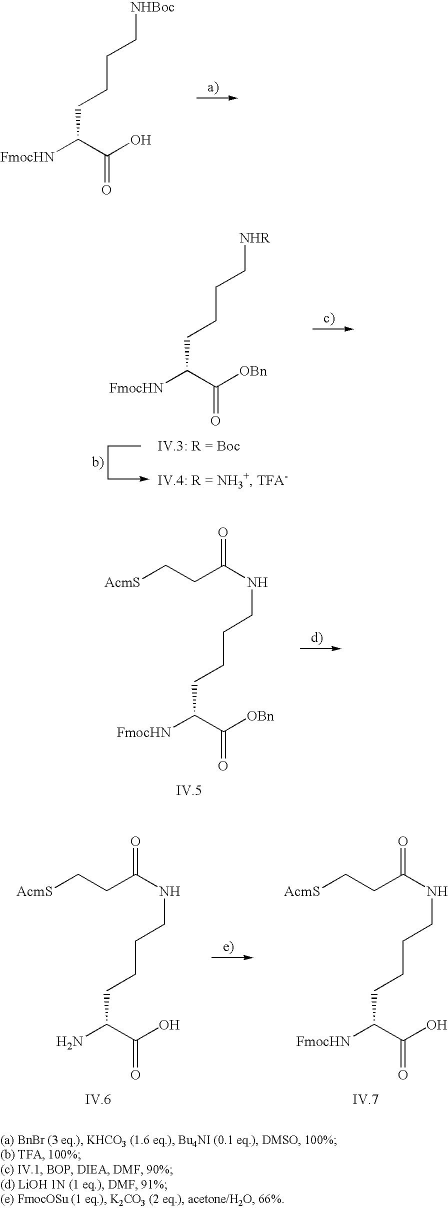

This monomer containing a protected thiol group is then incorporated into the straight chain hexapeptide IV.8 in the course of synthesis on a solid support.

The synthesis is done starting from the 2-chlorotrityl chloride resin according to the Fmoc strategy as illustrated hereinafter:

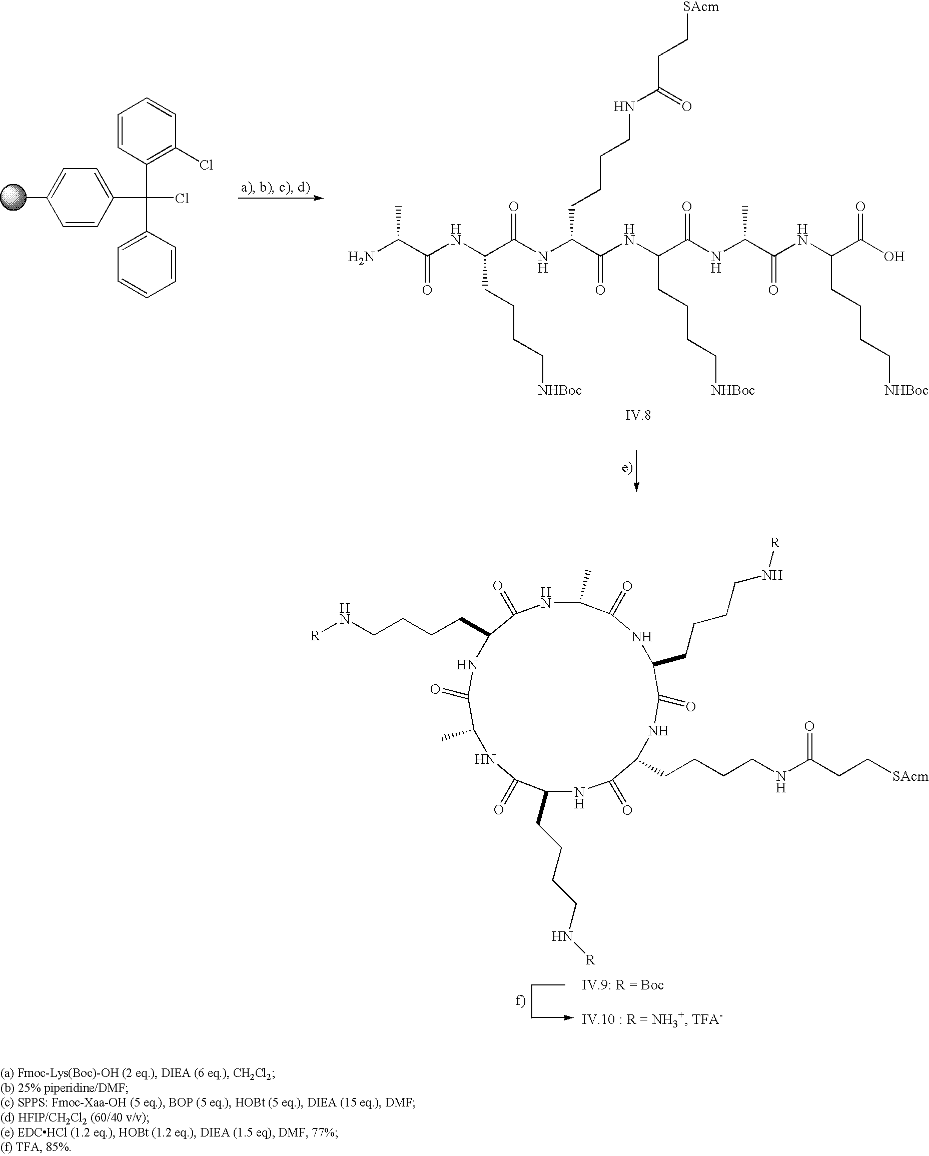

Cyclization of the straight chain precursor IV.8 was found to be problematic. Early attempts of II.2 macrocycle synthesis made under standard conditions have led to HPLC elution profiles of the crude macrocyclized product following deprotection associated with substantial quantities of polymerization products. A closer investigation of the macrocyclization conditions was hence required. To achieve this, we varied the type of coupling reagents, the concentration of the reaction medium as well as the reaction time interval. Results of this study are summarized into Table 1. In this table, the percentage of purity stands for the purity as calculated from the HPLC elution profile of crude Compound IV.10 obtained following treatment of IV.9 with TFA. In fact, it is not feasible to conduct an HPLC analysis of the fully protected cycle IV.9 since this compound is sparingly soluble into solvents typically used in HPLC.

TABLE 1
Various conditions used for the macrocyclization reaction

| | | Peptide | | Gross purity |
|---|---|---|---|---|
| | | concentration in | | level of |
| | Coupling reagents | the reaction | Reaction | Compound |
| Entry | used | mixture | time | IV.10 |
| 1 | EDC•HCl (1.2 eq), | 1 · 10⁻² M | 48 hours | 8% |
| | HOBt (1.2 eq), | | | |
| | DIEA (1.5 eq) | | | |
| 2 | EDC•HCl (1.2 eq), | 1.5 · 10⁻³ M | 72 hours | 17% |
| | HOBt (1.2 eq), | | | |
| | DIEA (1.5 eq) | | | |
| 3 | EDC•HCl (1.2 eq), | 4 · 10⁻³ M | 48 hours | 8% |
| | HOBt (1.2 eq), | | | |
| | DIEA (1.5 eq) | | | |
| 4 | EDC•HCl (1.2 eq), | 4 · 10⁻³ M | 72 hours | 63% |
| | HOBt (1.2 eq), | | | |
| | DIEA (1.5 eq) | | | |
| 5 | BOP (1.2 eq), | 4 · 10⁻³ M | 48 hours | 60% |
| | DIEA (1.5 eq) | | | |

It turns out that this reaction is very sensitive to the experimental conditions being used. In a first stage, it was observed that the concentration at which the reaction is run is a very critical factor. In fact, based on the dilution of the reaction medium, the purity of the macrocycle IV.10 compound varies to a great extent (entries #1, 2 and 4 of Table 1). A concentration of 4.10⁻³ M seems to be adapted for the formation of macrocycle IV.10 to occur with an acceptable purity. Conversely, the outcome of the reaction is greatly influenced by the particular coupling reagent being used as well as by the pH at which the reaction takes place (pH˜7 for EDC.HCl coupling, pH˜8-9 for BOP coupling). In fact, the BOP coupling reagent yields clearly poorer results as compared to EDC.HCl (entries #4 and 5). Finally, the reaction time interval was evaluated. In the case of EDC.HCl. coupling, 72 hours are required for full macrocyclization to occur (entries #3 and 4).

Differences in reactivity were also observed depending on the temperature at which the reaction occurs. At a temperature of 40° C., polymerization products are highly predominant .

The IV.10 macrocycle compound thus obtained is coupled to a protected pentapeptide Boc-Lys(Boc)-Gly-Tyr(OtBu)-Tyr(OtBu)-Ahx-OH P1 in the presence of BOP with no further purification resulting into a fully protected trimeric ligand. Boc and tBu as well as Acm protecting groups are removed in one single step and the L38 ligand having a thiol functional group is purified by C₁₈ preparative HPLC.

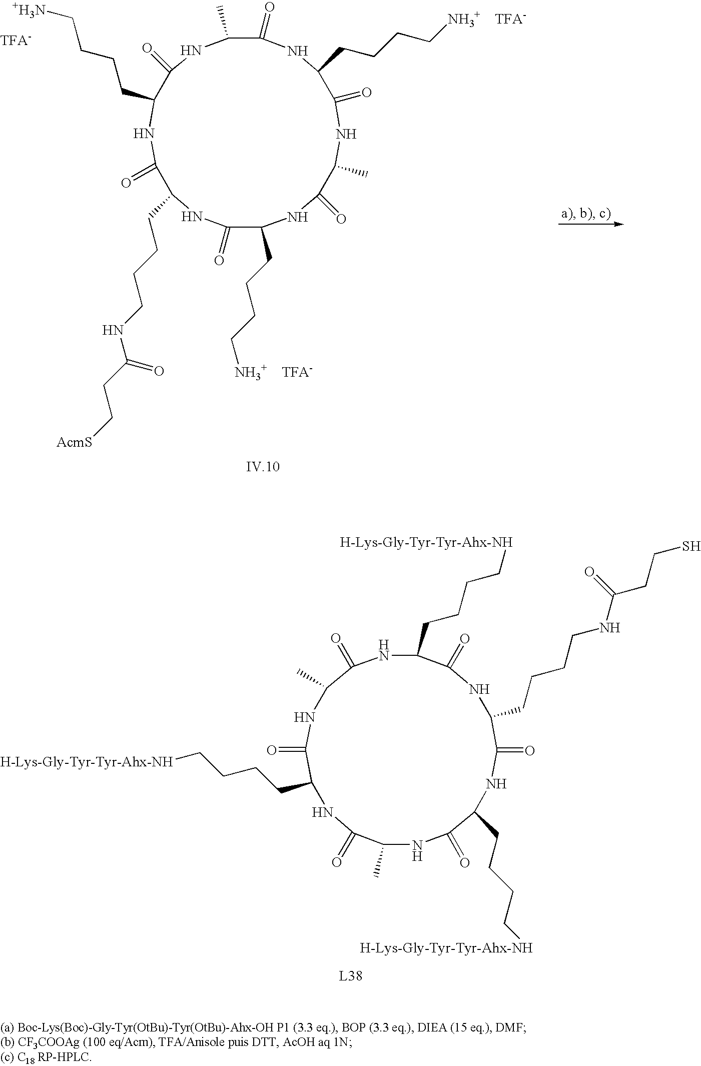

Basically, the acetamide group can be removed in various conditions some of which are orthogonal to the removal conditions of the Boc/tBu protecting groups. Mention should especially be made of the peptide treatment with mercury salts in an acid environment (Hg[OAc]₂ in a buffer solution at pH 4) (Veber, D.; Milkowski, J. D.; Varga, Varga, S. L.; Denkewalter, R. G.; Hirschmann, R. J. Am. Chem. Soc. 1972, 94, 5456-5461), the use of silver salts (e.g. CF₃SO₃Ag in the presence of anisole in trifluoroacetic acid) (Fujii, N.; Otaka, A.; Watanabe, T.; Okamachi, A.; Tamamura, H.; Yajima, H.; Inagaki, Y.; Nomizu, M.; Asano, K. J. Chem. Soc., Chem. Comm. 1989, 283-284), a treatment by thallium salts (TI(CF₃CO₂)₂) (Fujii, N. et al. Chem. Pharm. Bull. 1987, 36, 2339), or by iodine (Kamber, B. et al. Helv. Chim. Acta 1980, 63, 899) (in the last two cases a disulfide bond is formed).

It was decided to use silver trifluoroacetate CF₃CO₂Ag in trifluoroacetic acid in the presence of anisole. Such deprotection conditions allow the removal of both the acetamide protecting group and the tert-Butyloxycarbonyl (Boc) and tert-butyl ether (tBu) groups present on the peptide ((a) Albericio, F. et al. in Fmoc solid phase peptide synthesis: a practical approach, W. C. Chan and P. D. White (Eds.), Oxford University Press, Oxford, 2000, pp. 104; (b) Novabiochem catalog 2004/5 pp 3.21). The ligand is subsequently treated by a solution of 1,4-dithiothreitol (DTT) in acetic acid in order to reduce possible previously formed disulfide bonds

Following purification by preparative HPLC on a C₁₈ column, the L38 ligand is quickly submitted to a dimerization reaction.

b. Michael's Addition Reaction Between the Thiol Bearing Ligand and the Bismaleimide Spacer

Michael's addition reaction is done in a water/acetonitrile mixture. A solution of bismaleimide IV.1 (1 eq). in acetonitrile is added to a solution of the L38 ligand (3 eq.) in water. Even though Michael's addition reaction is fast, it is was decided to rather perform the reaction into a relatively dilute medium (2,5.10⁻³ M) in order to prevent the formation of possible disulfide bonds between two L38 ligands.

### Dimerization Driven by Michael's Addition Reaction of L38 to IV.1

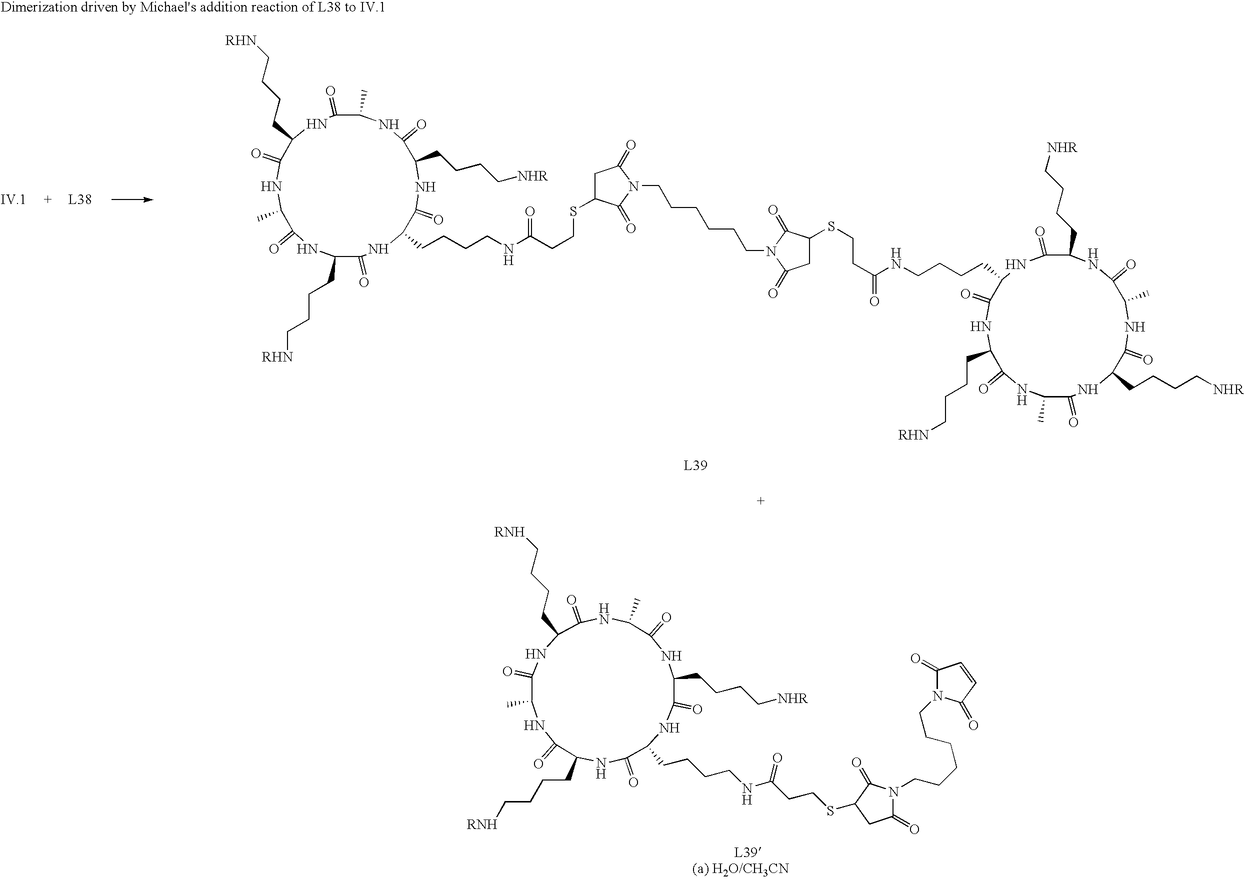

The reaction was monitored by analytical HPLC and it was noted after 15 minutes through the reaction, that a net proportion of monoadduct L39′ ligand was already formed. However, there was left some bismaleimide IV.1 and some thiol bearing ligand L38 added in excess. 24 hours later, the formation of a L39 dimerization product was detected. However, the reaction comes to completion only ten days later. It is noted that the entire portion of monoadduct compound has reacted with the L38 ligand added in excess to thereby yield the desired L39 dimer.

c. Conclusions

The key step of this synthesis strategy is to prepare a functionally substituted macrocycle. The outcome of this macrocyclization reaction has proven to be very sensitive to the choice of experimental conditions. Optimization of such conditions has somewhat led to a macrocycle with a fairly is acceptable purity so as to be used in subsequent synthesis steps without further purification.

Michael's addition reaction is simple to implement since it only requires that the ligand functionally substituted by a thiol group and the bismaleimide spacer be dissolved into a water/acetonitrile mixture. Adjusting the pH (pH˜7) can speed up the reaction and shorten the time of dimer formation. Purification of the crude reaction medium by preparative HPLC yields the dimerized ligand. However, some trouble in purifying the dimerization product was experienced when other bismaleimide compounds were used. Actually, this reaction was equally conducted starting from a polyethylene glycol IV.11 type bismaleimide spacer.

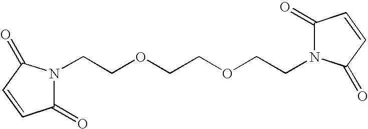

3) Ligand Dimerization Via the Click Chemistry Pathway

In parallel to the approach using Michael's reaction, dimerization of our ligands via a <<click chemistry>> strategy was contemplated.

a. <<click chemistry>> reaction

The 1,3-dipolar cycloaddition reaction between an alkyne and an azide was discovered in the 60′ s by Huisgen et al. (Huisgen, R.; Szeimies, G.; Moebius, L. Chem. Ber. 1967, 100, 2494-2507; Huisgen, R. Pure Appl. Chem. 1989, 61, 613-628). This reaction allows formation of triazole motifs according to the scheme outlined herein below:

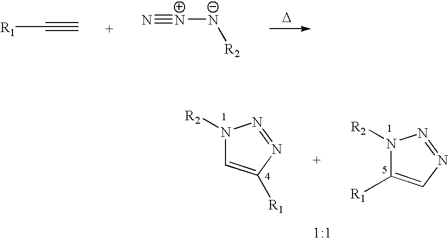

Recently, the team of Sharpless et al. has discovered that use of Copper I in catalytic amounts provides for milder reaction conditions (running the reaction at room temperature) and improves the reaction yield as well as selectivity (selective formation of 1,4-regioisomers) (Kolb, H. C.; Finn, M. G.; Sharpless, K. B. Angew. Chem. Int. Ed. 2001, 40, 2004-2021; Rostovtsev, V. V.; Green, L. G.; Fokin, V. V.; Sharpless, K. B. Angew. Chem. Int. Ed. 2002, 41, 2596-2599). This copper-catalyzed reaction allows a great flexibility in choosing the reaction conditions, whether at the solvent, pH or copper source level. Usually, the reaction is performed by means of Cu(SO₄).5H₂O which is reduced in situ by a reducing agent such as sodium ascorbate or ascorbic acid. However, in case such conditions fail to give the desired results, a source of copper I (CuI, CuBr, CuOTf.C₆H₆ . . . ) may be directly used. In the latter case, the reaction is more sensitive to oxidization and the formation of secondary products is sometimes observed. In order to prevent these unwanted side-reactions, the reaction should be carried out away from air and addition of a base such as 2,6-lutidine should be made.

In parallel to reports by Sharpless, the team of Meldal describes the use of copper I salts (CuI) for solid phase preparation of 1,4-triazole peptides starting from organic azide compounds and an alkyne bound to the endportion of a peptide-resin (Tornoe, C. W.; Christensen, C.; Meldal, M. J. Org. Chem. 2002, 67, 3057-3064). In a similar fashion to reactions in solution, this reaction accommodates all sorts of solvents (acetonitrile, N,N-dimethylformamide, toluene, dichloromethane) and gives readily access to the desired triazole peptide with an excellent purity.

Since this discovery, the 1,3-dipolar reaction of Huisgen is currently used in a number of application fields: whether in combinatory chemistry (Löber, S.; Rodriguez-Loaiza, P.; Gmeiner, P. Org. Lett. 2003, 5, 1753-1755; Kolb, H. C. et al. Preparation of 1,2,3-triazole carboxylic acids from azides and β-ketoesters in the presence of base. US2003135050), or in organic chemistry ((a) Fazio, F.; Bryan, M. C.; Blixt, O.; Paulson, J. C.; Wong, C.-H. J. Am. Chem. Soc. 2002, 124, 14397-14402; (b) Bodine, K. D.; Gin, D. Y.; Gin, M. S. J. Am. Chem. Soc. 2004, 126, 1638-1639; (c) Seo, T. S.; Li, Z.; Ruparel, H.; Ju, J. J. Org. Chem. 2003, 68, 609-612; (d) Zhou, Z.; Fahrni, C. J. J. Am. Chem. Soc. 2004, 126, 8862-8863; (e) Jin, T.; Kamijo, S.; Yamamoto, Y. Eur. J. Org. Chem. 2004, 3789-3791) or still in bioconjugate synthesis ((a) Wang, Q.; Chan, T. R.; Hilgraf, R.; Fokin, V. V.; Sharpless, K. B.; Finn , M. G. J. Am. Chem. Soc. 2003, 125, 3192-3193. (b) Link, A. J.; Tirell, D. A. J. Am. Chem. Soc. 2003, 125, 11164-11165. (c) Speers, A. E.; Adam, G. C.; Cravatt, B. F. J. Am. Chem. Soc. 2003, 125, 4686-4687. (d) Seo, T. S.; Li, Z.; Ruparel, H.; Ju, J. J. Org. Chem. 2003, 68, 609-612. (e) Speers, A. E.; Cravatt, B. F. Chem. Biol. 2004, 11, 535-546. (f) Lee, L. V.; Mitchell, M. L.; Huang, S.-J.; Fokin, V. V.; Sharpless, K. B.; Wong, C.-H. J. Am. Chem. Soc. 2003, 125, 9588-8589).

b. Preparation of Two Reagents Required for the <<Click Chemistry>> Reaction: a Diazide and a Macrocycle Functionally Substituted by a Formal Alkyne

Diazide spacers are prepared in two steps starting from corresponding polyethylene glycol chains as follows:

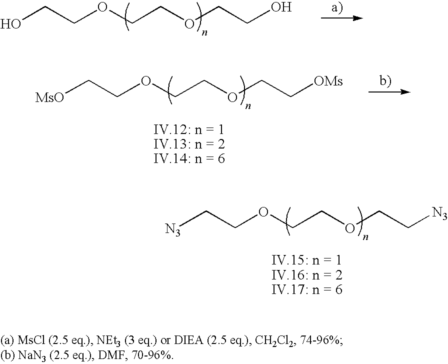

The diol is first converted into a dimesylate and the latter is next substituted by sodium azide addition. In order to assess the importance of the spacer's chain length, three diazide compounds of various sizes (IV.15 IV.17) were thus prepared (n=1, 2, 6).

### Preparation of a D,L-α-Peptide Macrocycle for the Synthesis of a D-Lysine IV.21 derivative including the formal alkyne functionality and adequately protected for use in solid support peptide synthesis

The formal alkyne derivative (IV.18) used for constructing IV.21 and generating the macrocycle functionality, stems from the reaction between succinic anhydride and mono-propargylamine as follows:

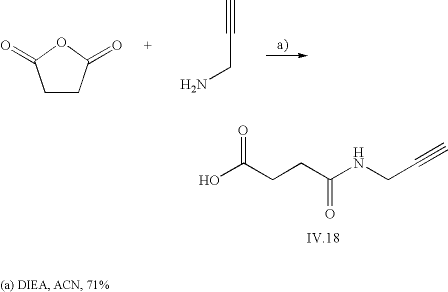

The N-prop-2-ynyl-succinamic acid (IV.18) thus obtained is then coupled to the N-Fmoc-protected D-lysine amino acid in order to form the IV.19 derivative. A series of protection and deprotection steps provide the IV.21 precursor in 5 steps with an overall yield of 23% .

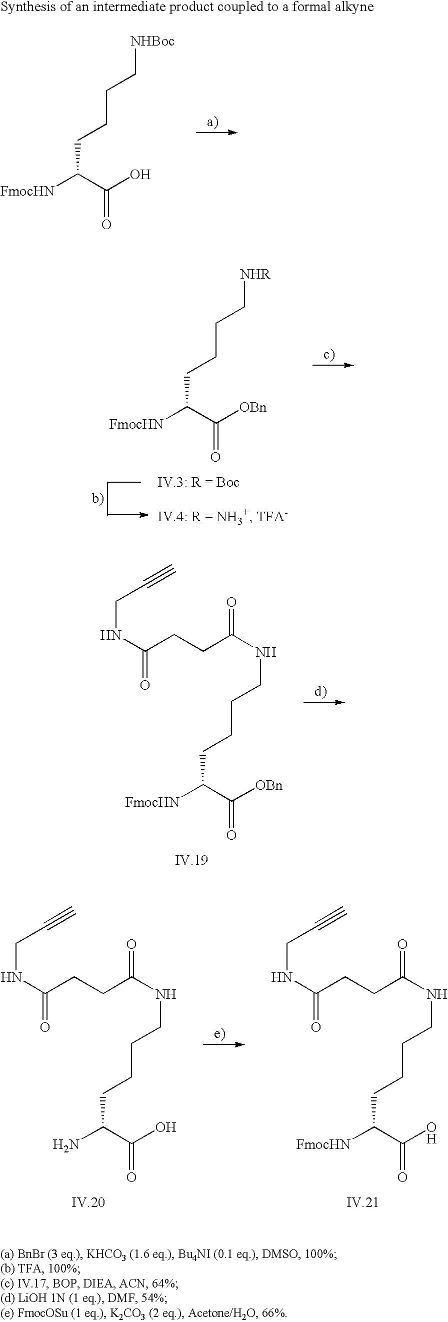

The functionally substituted precursor thus obtained is then incorporated into the straight chain IV.22 hexapeptide during solid phase synthesis. The peptide is subsequently cyclized in order to form the IV.23 macrocycle.

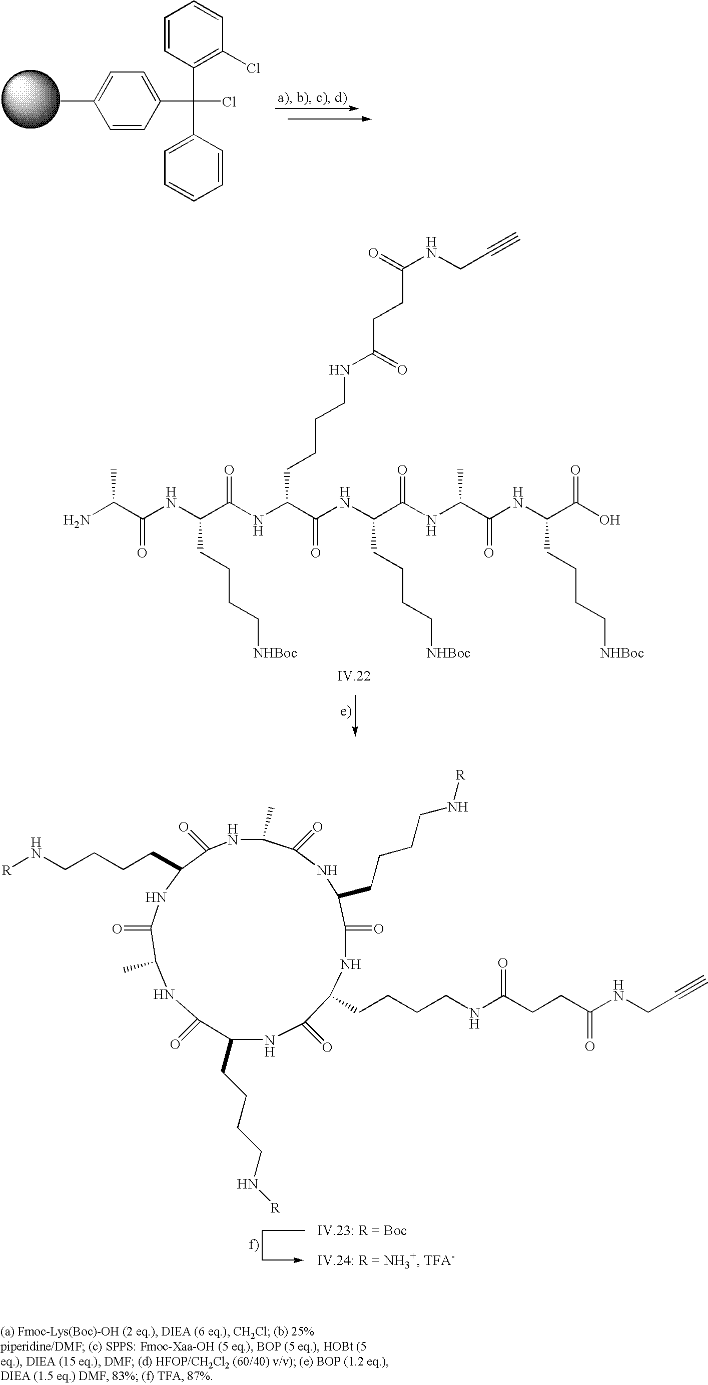

Again, the macrocyclization reaction had to be optimized. In fact, the HPLC elution profile of the cyclization reaction conducted by means of an EDC.HCl type coupling reagent shows a narrow peak corresponding to the deprotected macrocycle IV.24, but this is associated with a broad peak corresponding to polymerization products. Even though, the dilution of the reaction medium increased the purity of the macrocycle compound, the reaction yield with this coupling reagent is still very low.

As a result, it was contemplated to use the BOP reagent. This resulted into a substantial increase of the yield (83%) and the macrocycle purity (67%) following deprotection of the Boc groups.

By optimizing the cyclization reaction conditions, it was possible to attempt a peptide coupling reaction in order to form the corresponding L40 ligand without prior purification of the IV.24 macrocycle compound by preparative HPLC.

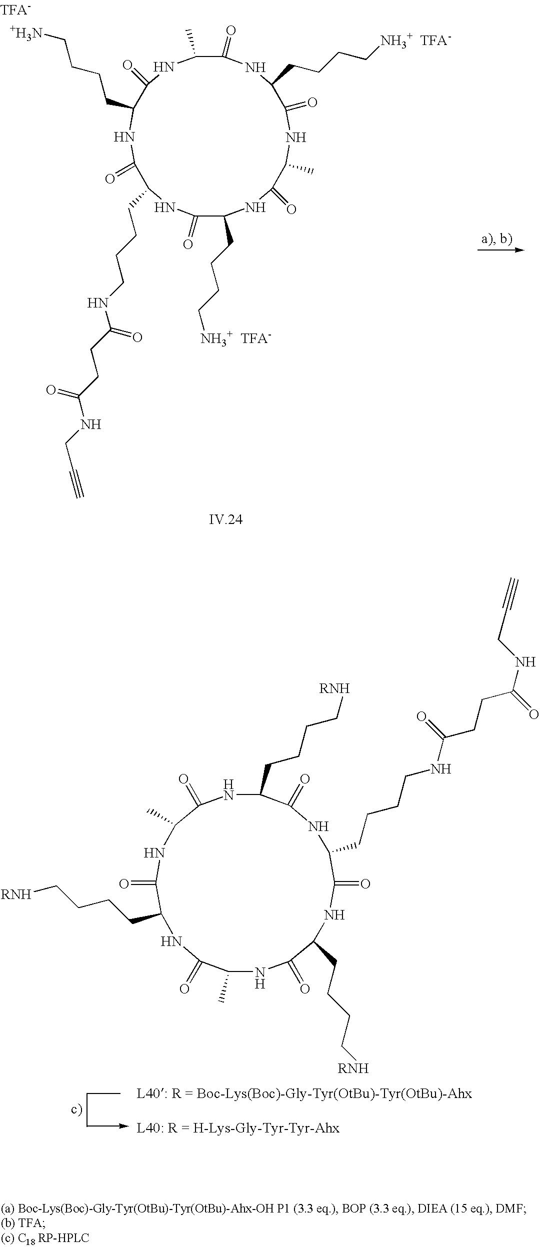

c. <<Click Chemistry>> Reaction

i. Refining the <<Click Chemistry>> Reaction

The <<click chemistry>> reaction was first considered starting from either the deprotected (L40) or non deprotected (L40′) ligand from these protecting groups. Reactivity testing was done with the IV.15 diazide .

Table 2 summarizes the experimental conditions used. The progress of the reaction is monitored by analytical HPLC either by direct injection of the crude reaction medium in the case of experiments made with the deprotected ligand (L40) or after prior treatment with trifluoroacetic acid in the case of reactions made with the protected ligand (L40′). As a matter of fact, the low solubility of L40′ ligand seriously complicates its analysis by reverse phase HPLC and direct monitoring of the dimerization reaction driven by <<click chemistry>>.

Initially, use was made of the standard conditions described by Sharpless is namely a source of copper II, Cu(SO₄).5H₂O, introduced in catalytic amounts and reduced in situ by a reducing agent, sodium ascorbate. The L40 ligand which is very hydrophilic was dissolved into a tBuOH/H₂O (1:1, v/v) mixture, typically used by Sharpless. Reactions performed with the protected L40′ ligand were conducted in N,N-dimethylformamide, the only solvent in which it is soluble .

After the reaction was run for four days, no change was noted by HPLC, whatever the particular starting ligand used (L40 or L40′) despite adding extra copper II and reducing agent. The reaction was also conducted at 80° C. and under micro-waving. However, no result was seen: Instead, breakdown of the starting product was observed .

TABLE 2
Experimental conditions used for the 1,3-dipolar reaction
starting from the L40 or L40′ ligands

| Entry | Ligand | Conditions | Solvent |
|---|---|---|---|
| 1 | L40′ | Cu(SO)₄•5H₂O (0.02 eq.) | DMF |
| | | sodium Ascorbate (0.2 eq.) | |
| 2 | L40′ | Cul (13 eq.), ascorbic acid (7 eq.), | DMF/Pyridine |
| | | DIEA (17 eq.) | |
| 3 | L40′ | Cul (2 eq.), 2,6-lutidine (2 eq.), | DMF |
| | | DIEA (2 eq.) | |
| 4 | L40 | Cu(SO)₄•5H₂O (0.02 eq.) | tBuOH/H₂O |
| | | Ascorbate de sodium (0.2 eq.) | |
| 5 | L40 | Cul (13 eq.), ascorbic acid (7 eq.), | DMF/Pyridine |
| | | DIEA (17 eq) | |
| 6 | L40 | Cul (2 eq.), 2,6-lutidine (2 eq.), | DMF |
| | | DIEA (2 eq.) | |

Direct use of a copper I source was hence contemplated based on the work of Kirschenbaum's (Jang, H.; Fafarman, A.; Holub, J. M.; Kirschenbaum, K. Org. Lett. 2005, 7, 1951-1954) and Ghadiri's teams (Van Maarseveen, J. H.; Home, W. S., Ghadiri M. R. Org. Lett. 2005, 7, 4503-4506), who conducted 1,3-dipolar reactions starting from peptides either in solution or in solid phase, by means of copper iodide (CuI). Use of a copper I source required lab work to be conducted under an inert atmosphere and flushing of the solvent with argon following solubilization of the product. Despite these precautions, no change was observed when running the reaction starting from ligands L40 and L40′.

One likely possibility for this lack of reactivity is the high proportion of free amine groups present on the L40 ligand. In fact, one possibility is that copper might be chelated around these functional groups thus preventing it from chelating the alkyne functionality present on the macrocycle. This explanation has already been suggested by Nolte et al. in the case of reactions conducted with peptides containing arginine residues (Dirks, A. J.; Van Berkel, S. S.; Hatzakis, N. S.; Opsteen, J. A.; Van Delft, F. L.; Cornelissen, J. J. L. M.; Rowan, A. E.; Van Hest, J. C. M.; Rutjes, F. P. J. T.; Nolte, R. J. M.; Chem. Commun. 2005, 4172-4174). Alternatively, the severe steric hindrance related to the presence of the pentapeptide on the macrocycle's spacer might prevent or hinder copper chelation.

As a result of these problems, it was decided to realize the <<click chemistry>> reaction starting from the protected (IV.23) or non protected (IV.24) macrocycle. Table 3 gives the experimental conditions used in the course of reacting macrocycles with diazide compounds IV.15. As mentioned previously, the progress of the reaction is monitored by analytical HPLC either by direct injection of the reaction mixture in the case of experiments performed on the deprotected macrocycle (IV.24) or following prior treatment with trifluoroacetic acid in the case of a reaction performed starting from the protected macrocycle (IV.23).

When working according to conditions of Kirschenbaum, no change was observed by analytical HPLC (entry #1). By contrast, when working according to conditions of Ghadiri, (entries #2, 3 and 4), there was observed a substantial decrease of the starting product coupled with the formation of a new product, However, this product could not be characterized at this stage. These reactions is were conducted with acetonitrile (entries #2 and 4) or into a water/acetonitrile mixture (entry #3). This solvent was chosen because it is recommended by Sharpless when using a copper I source (Rostovtsev, V. V.; Green, L. G.; Fokin, V. V.; Sharpless, K. B. Angew. Chem. Int. Ed. 2002, 41, 2596-2599).

TABLE 3
Experimental conditions used for the 1,3-dipolar reaction
starting from the protected or non protected macrocycle

| Entry | Macrocycle | Conditions | Solvent |
|---|---|---|---|
| 1 | IV.24 | Cul (13 eq.), ascorbic acid (7 eq.), | DMF/ |
| | | DIEA (17 eq.) | Pyridine |
| 2 | IV.24 | Cul (2 eq.), 2,6-lutidine (2 eq.), | CH₃CN |
| | | DIEA (2 eq.) | |
| 3 | IV.24 | Cul (2 eq.), 2,6-lutidine (2 eq.), | H₂O/CH₃CN |
| | | DIEA (2 eq.) | |
| 4 | IV.23 | Cul (2 eq.), 2,6-lutidine (2 eq.), | CH₃CN |
| | | DIEA (2 eq.) | |
| 5 | IV.23 | CuBr (0.2 eq.), DBU (3 eq.), 80° C. | THF/CH₃CN |
| 6 | IV.23 | Cul (2 eq.), 2,6-lutidine (25 eq.), | DMF |
| | | DIEA (25 eq.) | |

Based on these results, it was decided to perform the reaction starting from the protected macrocycle IV.24 in N,N′-dimethylformamide to achieve better solubilization. In peptide-like compound synthesis, Meldal uses 2 equivalents of CuI and 50 equivalents of DIEA (Tornoe, C. W.; Christensen, C.; Meldal, M. J. Org. Chem. 2002, 67, 3057-3064). In this instance, the proportion of base was increased to 25 equivalents of DIEA and 25 equivalents of 2,6-lutidine (entry #6). After stirring for 3 days and treatment with TFA, HPLC analysis reveals the nearly total disappearance of the starting ring compound (converted into IV.24 following a TFA treatment, with about 2% still present) and formation of a major product. Purification of the crude reaction medium by preparative HPLC identified the major product as being the expected dimeric molecule IV.25 derived from the click chemistry>> reaction. According to HPLC analysis, the IV.24/IV.25 ratio is 3/97 (FIG. 2).

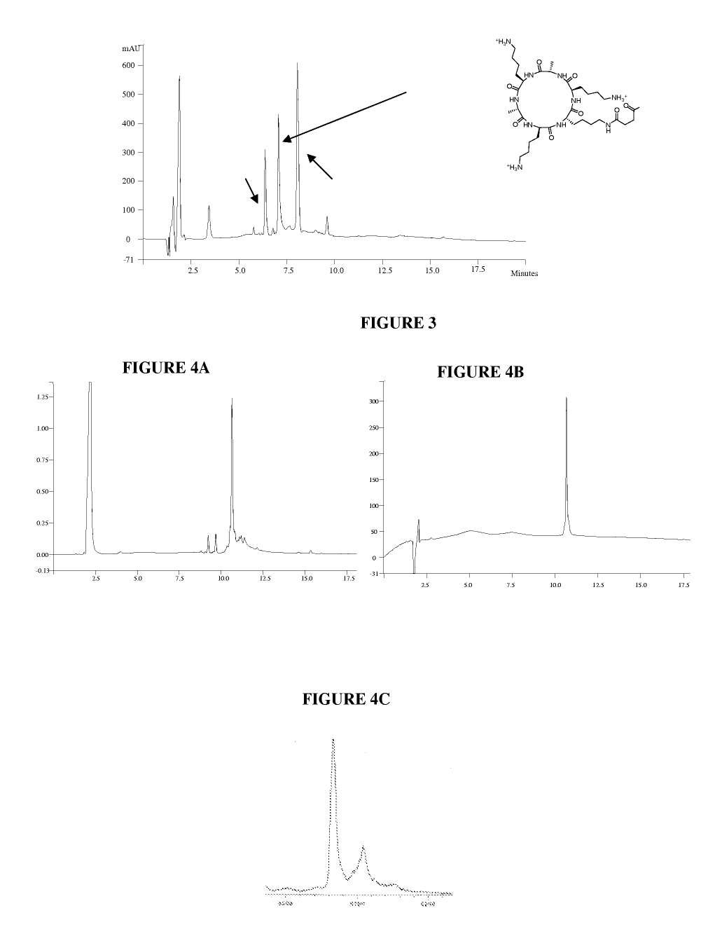

The use of CuBr (entry #5) was also investigated. After stirring for 3 days at 80° C., the reaction was stopped. As reported previously, HPLC analysis shows a nearly total conversion of the starting product (12% of IV.24 still present) but this time two products are formed in major proportions according to a 41/59 (IV.25:IV.45) ratio. This is the expected dimerized IV.25 compound and the IV.25′ derivative resulting from the formation of one single triazole motif (FIG. 3)

While these conditions give access to a dimerization product, they are less efficient than those involving copper iodide since the reaction ends up with a mixture of three products 3 days later. Consequently, conditions described in entry #6 of Table 2 were definitely adopted to generalize the 1,3-dipolar reaction to other spacers IV.16 and IV.17.

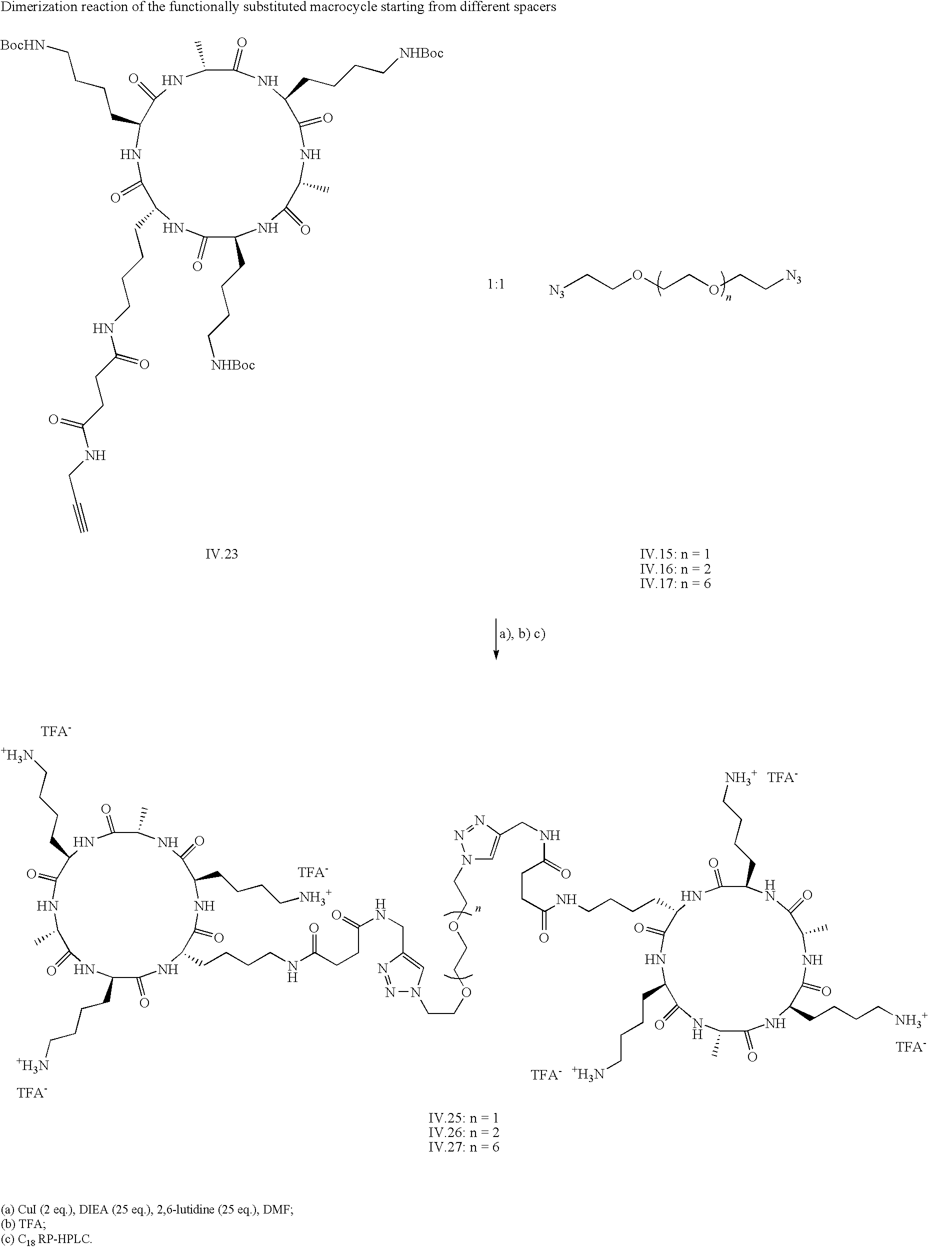

Compounds IV.25 to IV.27 were isolated in yields ranging from 12 to 20% following preparative HPLC purification.

Corresponding ligands L41 to L43 were then prepared by coupling dimerized macrocycles to the protected pentapeptide Boc-Lys(Boc)-Gly-Tyr(OtBu)-Tyr(OtBu)-Ahx-OH(P1), deprotection by removal of protecting groups and purification by preparative HPLC.

Usually, the coupling reaction is conducted within two days. In this instance, stirring for one week at room temperature is required for the reaction to come to completion. Stopping the reaction merely three days later shows the presence of several intermediate products resulting from partial coupling of the pentapeptide to available ^{ε}NH amines.

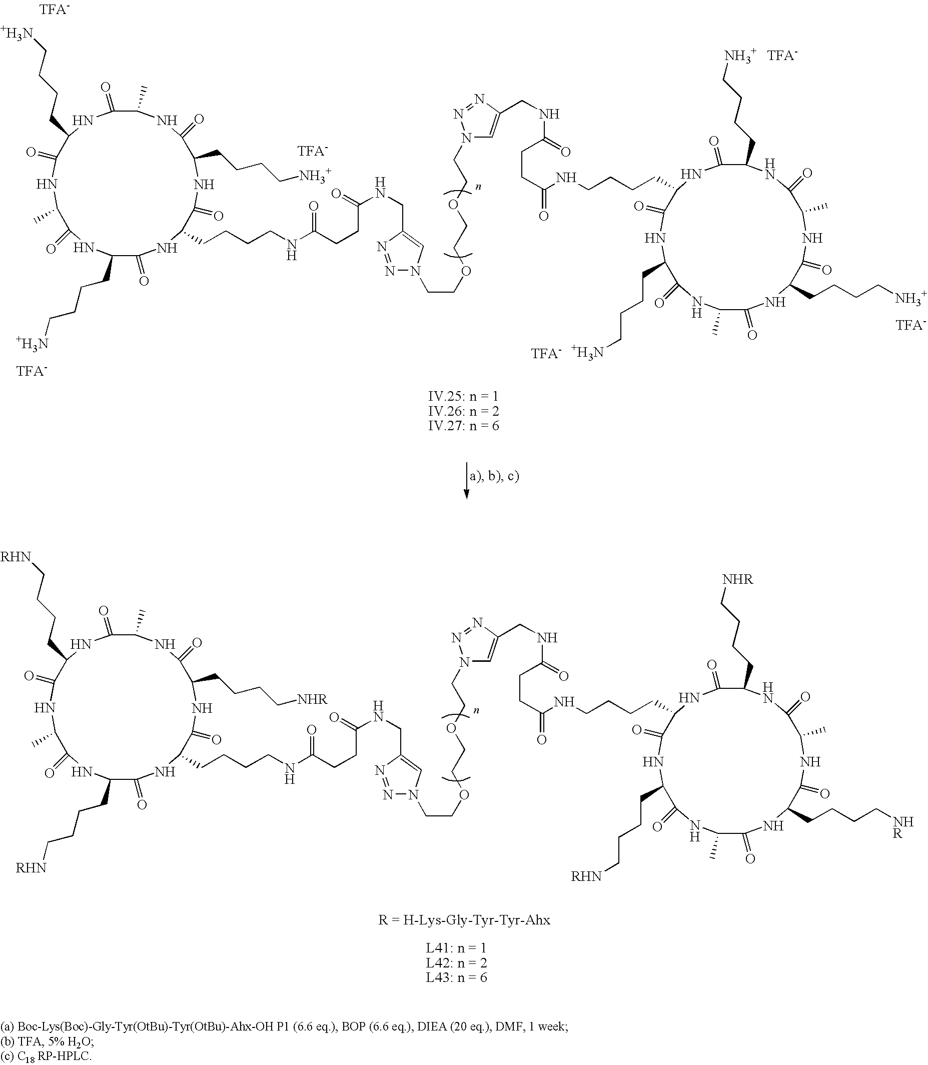

Each of these ligands was characterized by HPLC, mass spectrometry (MALDI-TOF) (FIGS. 4A, 4B and 4C).

TABLE 4
Analysis of ligand L41 by NMR. NMR experiments performed
in H₂O/tert-Butanol-d₉ at 300K with a 500 MHz spectrometer

| | NH | CHα | CHβ | CHγ | others |
|---|---|---|---|---|---|
| Residues | (ppm) | (ppm) | (ppm) | (ppm) | (ppm) |
| Lys¹⁴³ | — | 4.09 | 1.99 | 1.56 | ^{δ}CH 1.81; |
| | | | | | ^{ε}CH 3.08 |
| Gly¹⁴⁴ | — | 4.09 | — | — | — |
| Tyr¹⁴⁵ | 8.23 | 4.63 | 2.96 | — | — |
| Tyr¹⁴⁶ | 8.03 | 4.49 | 2.97 | — | — |
| Ahx | 7.47 | 2.23 | 1.55 | 1.14 | ^{δ}CH 1.37; ^{ε}CH |
| | | | | | 3.03/3.20 |
| Lysine | 8.33/8.23 | 4.31 | 1.78/1.74 | 1.42/1.4 | ^{δ}CH 1.56/1.54; |
| (core) | (α) | | | | ^{ε}CH 3.21 |
| | 7.95/7.91 (ε) | | | | |
| D-Alanine | 8.33 | 4.45 | 1.41 | — | — |
| (core) | | | | | |

ii. Notes on the <<Click Chemistry>> Reaction

The order in which the reagents are added seems to be important in the previously technically refined reaction. In fact, both bases should be added prior to addition of copper iodide. If the metal is added first, a decrease of the dimerized macrocycle purity is observed at the end of the reaction.

Furthermore, it is interesting to note that the use of two equivalents of a functionally substituted ring with respect to the diazide is not necessary for dimer formation. In fact, by preparing an equimolecular mixture, the dimerization reaction comes to completion too. It is hence believed, as already noted by Sharpless, that the use of a diazide favours the formation of the ditriazole and is that the formation of the first triazole promotes the formation of the second one (Rodionov, V. O.; Fokin, V. V.; Finn, M. G. Angew. Chem. Int. Ed. 2005, 44, 2210-2215).

Finally, the use of an additive such the tris-(benzyltriazolylmethyl) amine (TBTA) may be the right option to perform a <<click chemistry>> reaction directly from the L40 or L40′ ligand ((a) Wang, Q.; Chan, T. R.; Hilgraf, R.; Fokin, V. V.; Sharpless, K. B.; Finn , M. G. J. Am. Chem. Soc. 2003, 125, 3192-3193. (b) Chan, T. R.; Hilgraf R.; Sharpless K. B.; Fokin, V. V. Org. Lett. 2004, 6, 2853-2855). As pointed out by Sharpless, TBTA encloses copper I and prevents destabilizing interactions originating from free binding sites such as amines. Moreover, it speeds up the 1,3-dipolar cycloaddition reaction.

### B) Biology

1) Purification and Culturing of Spleen B Cells

Spleens were surgically removed from BALB/c mice aged from 5 to 12 weeks. Spleen B cells were prepared by positive selection using magnetic beads coated with monoclonal anti-CD19 antibodies (MACS, Milteny biotech, Germany). This fraction contains over 95% of B220⁺ cells. B cells (3×10⁶/ml) were next cultured in RPMI 1640 medium supplemented with 10% complement depleted FBS, gentamycin (10 μg/ml), 25 mM HEPES and 10 mM β-mercaptoethanol in presence of CD40L analogs. Cells were pulsed with 1 μCi/well of tritiated thymidine (ICN, Irvine, Calif.) for the last 20 hours of culturing and the uptake of [³H]-thymidine as expressed in cpm was measured after 72 hours using a Matrix 9600 model direct 13 counter (Packard, Meriden, Conn.). Results (FIG. 5B) are expressed as a stimulation index corresponding to cpm obtained for cultures with CD40L analogs divided by cpm obtained for cultures without CD40L analogs. Mice used in this study were raised in our animal housing facilities which are licensed by the french veterinary administration under number D-67-482-2.

2) Cell Culture and Induction of Apoptosis

Burkitt's lymphoma (RAJI) was cultured in RPMI 1640 medium (Cambrex Bioscience, Verviers, Belgium) supplemented with 10% decomplemented foetal calf serum (FBS) and gentamycin (10 μg/ml). For assaying apoptosis, cells (1×10⁶/ml) were incubated at 37° C. in 96-well plates, at the specified concentrations of CD40L analogs, in a final volume of 200 μl. After incubation for 16 hours, cell apoptosis was measured as described herein below.

3) Cell Apoptosis Measurement

Apoptosis was assayed by measuring the drop in mitochondrial transmembrane potential (Δψₘ) associated with a reduction in level of cationic colouring agent 3,3′-dihexyoxacarbocyanine iodide (DiOC₆(3)) capture, as demonstrated by flow cytometry (Zamzami et al., J. Exp. Med. 1995, 191:1661).

To this end, cells were pulsed with DiOC₆(3) (Interchim, Montluçon, France)) at a final concentration of 40 nM for the last 45 minutes of culturing.

Cells were subsequently washed, resuspended in 300 μl of PBS and analyzed by flow cytometry. Results (FIG. 5A) are expressed in percentage of specific apoptosis according the following formula: % of specific apoptosis=[(% of dead treated cells−% of dead control cells)×100]/(100−% de dead control cells).

Cells were analyzed by a FACSCalibur® instrument. At least 25000 pulse is events were acquired for each experiment using the CellQuest 3.3 software (Becton Dickinson, Pont de Claix, France) and data were analyzed using the WinMDI 2.8 software (Joseph Trotter, Scripps Research Institute, http://facs.scripps.edu/software.html).

4) Results

II was observed that the L41 ligand, a dimeric ligand as afforded by <<click chemistry>> synthesis, induces a Burkitt lymphoma (RAJI) specific apoptosis percentage at dose levels where no activity was seen for L1 (FIG. 5A). In addition, this ligand induces alone murine spleen B cell proliferation whereas no such an effect was noted for the monomeric ligand L1 in the absence of an anti-CD40 3/23 antibody potentiating effect (FIG. 5B).

### Detailed Experimental Section

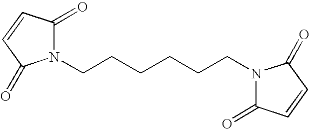

### Bismaleimidehexane (IV.1)

N-(methoxy-carbonyl)maleimide (320 mg; 2.06 mmol) is added at 0° C. to a stirred solution of N,N′-diaminohexane (100 mg; 0.86 mmol) in a saturated NaHCO₃/THF (tetrahydrofuran) mixture) (Vₜ=8 ml, 1:1 v/v). The reaction mixture is stirred at 0° C. for 10 minutes and thereafter stirred for 4 hours at room temperature. The product is next recovered by extraction with ethyl acetate. The pooled organic layers were washed with water and brine, dried over Na₂SO₄, filtered and concentrated under vacuum to thereby obtain Compound VI.1 (230 mg; yield=100%). HPLC t_{R} 15.78 min. (linear gradient, 30-100% B, 20 min); ¹H NMR (300 MHz, CDCl₃, 298 K) δ 6.67 (s; CH maleimide; 4H); 3.48 (t; NCH₂; J=7.2 Hz; 4H); 1.62-1.54 (m, NCH₂CH₂, 4H); 1.31-1.26 (m, NCH₂CH₂CH₂, 4H); ¹³C NMR (300 MHz, CDCl₃, 298 K) 170.84 (4 CO); 134.03 (4 CH); 37.67 (2 CH₂); 28.32 (2 CH₂); 26.15 (2 CH₂); HRMS (ESI): m/z: Calculated for C₁₄H₁₆N₂O₄Na: 277.11. Found: 277.1183; Calculated for C₁₄H₁₆N₂O₄Na: 299.11. Found: 299.1002.

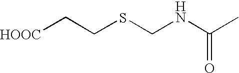

### 3-((acetamidomethyl)sulphanyl)propanoic acid (Acm) (IV.2)

3-mercaptopropanoic acid (1.64 ml; 19 mmol) is dissolved into trifluoroacetic acid (30 ml). N-(hydroxymethyl)acetamide (1.6 g; 19 mmol) is next added. After stirring for 30 minutes at room temperature, TFA is evaporated off to obtain IV.2: HPLC t_{R} 7.1 min. (linear gradient, 5-65% B, 20 min); ¹H NMR (300 MHz, CDCl₃, 298 K) δ 12.4 (s, COOH, 1H); 7.74 (d, NH, J=5.8 Hz, 1H); 4.34 (d, SCH₂NH, J=6 Hz, 2H); 2.81 (t, CH₂SCH₂NH, J=6.4 Hz, 2H); 2.67 (t, CH₂COOH, J=6.4 Hz, 2H); 2.1 (s, NHCOCH₃, 3H); ¹³C NMR (300 MHz, CDCl₃, 298 K) 177.45 (CO); 174.63 (CO); 41.60 (CH₂); 34.37 (CH₂); 25.67 (CH₂); 21.46 (CH₃).

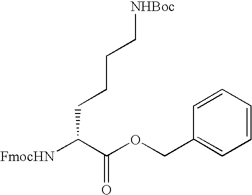

### Fmoc-D-Lys(Boc)-OBn (IV.3)

Fmoc-D-Lys(Boc)-OH (5 g; 10 mmol) is dissolved into DMSO (dimethylsulphoxide) (22 ml). KHCO₃ (1.6 g; 16 mmol) and Bu₄NI (390 mg; 1 mmol) are added in succession. After stirring for 30 min., benzyl bromide is added (3.8 ml; 30 mmol). The mixture is then stirred overnight. Water is added to the solution and the aqueous layer is extracted with ethyl acetate. The pooled organic layers are washed with a saturated solution of NaHCO₃, a saturated is solution of Na₂S₂O₃ and brine. Evaporation of the solvent and precipitation in cyclohexane at 78° C. yields Compound IV.3 (5.80 g; yield=98%): HPLC t_{R} 16.94 min. (linear gradient, 30-100% B, 20 min); ¹H NMR (300 MHz, CDCl₃, 298 K) δ7.76 (d, CH Arom Fmoc, J=7.4 Hz, 2H); 7.60 (d, CH Arom Fmoc, J=7.4 Hz, 2H); 7.42-7.25 (m, CH Arom Fmoc and Ph, 9H); 5.18 (s, CH₂Ph, 2H); 4.47-4.23 (m, CH₂ Fmoc and CH Fmoc, 3H); 4.21 (t, FmocNHCH, J=6.9 Hz, 1H); 3.09-3.01 (m, CH₂ Lysine, 2H); 1.95-1.64 (m, CH₂ Lysine, 2H); 1.51-1.22 (m, CH₂ Lysine and C(CH₃)₃, 13H); ¹³C NMR (300 MHz, CDCl₃, 298 K) 172.33 (CO); 156.08 (CO); 156.00 (CO); 143.93 (2 C); 141.3 (C); 135.27 (2 C); 128.65 (2 CH); 128.54 (2 CH); 128.37 (CH); 127.71 (2 CH); 127.08 (2 CH); 125.12 (2 CH); 119.98 (2 CH); 79.20 (C); 67.19 (CH₂); 67.03 (CH₂); 53.80 (CH); 47.16 (CH); 40.04 (CH₂); 32.11 (CH₂); 29.58 (CH₂); 28.43 (CH₃); 22.30 (C₂).

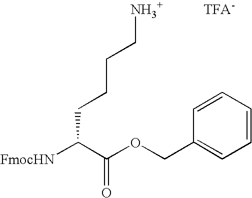

### Fmoc-D-Lys-OBn salt, TFA (IV.4)

Compound IV.3 (5.80 g; 10.34 mmol) is dissolved into TFA (16 ml). After stirring for 30 minutes at room temperature, TFA is evaporated off using ether and cyclohexane. Compound IV.4 is thereby obtained: HPLC t_{R} 10.91 min. (linear gradient, 30-100% B, 20 min); ¹H NMR (300 MHz, CDCl₃, 298 K) δ 7.71 (d, CH Arom Fmoc, J=7.5 Hz, 2H); 7.53 (d, CH Arom Fmoc, J=7.5 Hz, 2H); 7.37-7.21 (m, CH Arom Fmoc and Ph, 9H); 5.6 (d, NH, J=8.06 Hz, 1H); 5.12 (s, CH₂Ph, 2H); 4.33 (m, CH Fmoc and CH₂ Fmoc, 3H); 4.14 (t, FmocNHCH, J=6.94 Hz, 1H); 2.91-2.83 (m, CH₂ Lysine, 2H); 1.65-1.52 (m, CH₂ Lysine, 4H); 1.39-1.26 (m, CH₂ Lysine, 2H); NMR ¹³C (300 MHz, CDCl₃, 298 K) 172.12 (CO); 156.30 (CO); 143.66 2 (C); 141.26 (C); 135.08 (2 C); 128.63 (2 CH); 128.57 (2 CH); 128.33 (CH); 127.77 (CH); 127.10 (CH); 125.10 (CH); 120.01 (CH); 67.38 (CH₂); 67.21 (CH₂); 53.61 (CH); 47.01 (CH); 39.55 (CH₂); 31.76 (CH₂); 26.69 (CH₂); 21.98 (CH₂).

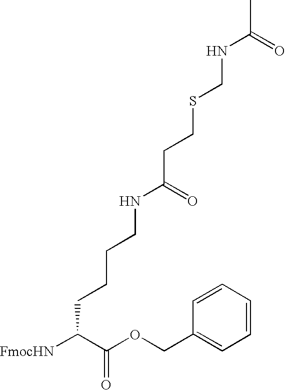

### (9H-fluoren-9-yl)methyl 1-((benzyloxy)carbonyl)-5-(3-((acetamidomethyl)sulphanyl)propanamido)pentylcarbamate (IV.5)

Compound IV.2 (2.2 g; 12.41 mmol) is dissolved into DMF (dimethylformamide) (40 ml). To a neutralized solution of TFA, addition is made of DIEA (N,N-diisopropylethyleneamine) (2 ml; 12.41 mmol). Next, there is added in succession compound IV.4 (10.34 mmol) dissolved into DMF (20 ml) with DIEA (2 ml; 12.41 mmol) and BOP (benzotriazol-1-yloxy-tris(dimethylamino)phosphonium hexafluorophosphate) (2.2 g; 12.41 mmol). The reaction mixture is adjusted to pH=8/9 with DIEA. After stirring for 6 hours, DMF is evaporated under reduced pressure and retaken up in CH₂Cl₂ previously washed several times with saturated NaHCO₃, water and 1N KHSO₄. Drying over Na₂SO₄ and evaporation provided an oil which was precipitated in CH₂Cl₂/IPE (diisopropyl ether) to yield the pure compound IV.5 (5.74 g; yield=90%): HPLC t_{R} 13.29 min. (linear gradient, 30-100% B, 20 min); HPLC t_{R} 13.29 min. (linear gradient, 30-100% B, 20 min.); ¹H NMR (300 MHz, DMSO-d₆, 298 K) δ8.46 (t, NH, J=6.11 Hz, 1H); 7.9 (d, CH Arom Fmoc, J=7.4 Hz, 2H); 7.87-7.81 (m, NH, 2H); 7.71 (d, CH Arom Fmoc, J=7.2 Hz, 2H); 7.45-7.26 (m, CH Arom Fmoc and Ph, 9H); 5.12 (s, CH₂Ph, 2H); 4.31 (d, CH Fmoc, J=6.3 Hz, 1H); 4.20 (d, CH₂ Fmoc, J=6.3 Hz, 2H); 4.06-4.01 (m, FmocNHCH, 1H); 3.0 (m, CH₂ Lysine, 2H); 2.72 (t, CH₂CONH, J=7.2 Hz, 2H); 2.53 (s, NHCOCH₃, 3H); 2.35 (t, CH₂SCH₂NHCOCH₃, J=7.2 Hz, 2H); 1.83 (s, SCH₂NH, 2H); 1.71-1.60 (m, CH₂ Lysine, 2H); 1.30-1.39 (m, CH₂ Lysine, 4H); ¹³C NMR (300 MHz, DMSO-d₆, 298 K) 172.78 (CO); 170.70 (CO); 169.68 (CO); 156.63 (CO); 144.25 (2 C); 141.17 (C); 136.40 (2 C); 128.66 (2 CH); 128.54 (2 CH); 128.33 (CH); 127.76 (2 CH); 127.11 (2 CH); 125.12 (2 CH); 120.11 (2 CH); 66.31 (CH₂); 66.12 (CH₂); 54.41 (CH); 47.07 (CH); 40.18 (CH₂); 38.66 (CH₂); 36.07 (CH₂); 30.72 (CH₂); 29.06 (CH₂); 26.62 (CH₂); 23.35 (CH₂); 23.01 (CH₃).

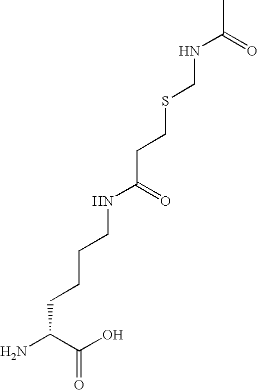

### 6-(3-((acetamidomethyl)sulphanyl)propanamido)-2-aminohexanoic acid (IV.6)

Compound IV.5 (4.6 g; 7.4 mmol) is dissolved into DMF (50 ml). A 1N solution of LiOH (22 ml) is gently added at 0° C. After stirring for 5 hours, DMF is evaporated under reduced pressure and precipitated in acetonitrile to provide compound IV.6 (1.91 g; yield=91%).

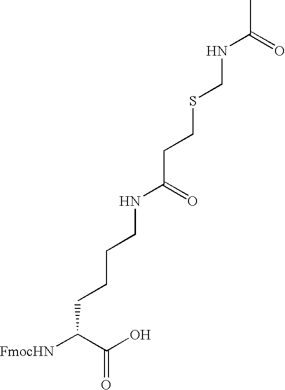

### Compound Fmoc-D-Lys(Acm)-OH (IV.7)

Compound IV.6 (2.21 g; 7.2 mmol) is dissolved into a mixed solution of H₂O (50 ml) and K₂CO₃ (2 g; 14.4 mmol). Next, Fmoc-OSu (9-fluorenylmethyloxycarbonyl-N-hydroxysuccinimide) (2.4 g; 7.2 mmol) in acetone (50 ml) is gently added. After stirring for 7 hours, water is added. The aqueous phase is quickly washed with ether and adjusted to pH=3 with 1N HCl and then extracted with CH₂Cl₂. Drying over Na₂SO₄ and evaporation of the filtrate provide an oil which is precipitated in CH₂Cl₂/IPE (diisopropyl ether) to afford the pure compound IV.7 (2.24 g; yield=60%): HPLC t_{R} 8.9 min (linear gradient, 30-100% B, 20 min) ¹H NMR (300 MHz, DMSO-d₆, 298 K) δ 8.47 (t, NH, J=6.07 Hz, 1H); 7.9 (d, CH Arom Fmoc, J=7.46 Hz, 2H); 7.85 (t, NH, J=5.92 Hz, 1H); 7.73 (d, CH Arom Fmoc, J=7.4 Hz, 2H); 7.58 (d, NH, J=8 Hz, 1H); 7.44-7.30 (m, CH Arom Fmoc, 4H); 4.26 (t, CH Fmoc, J=7.5 Hz, 1H); 4.20 (d, CH₂ Fmoc, J=6.3 Hz, 2H); 3.90 (m, FmocNHCH, 1H); 3.05-2.99 (m, CH₂ Lysine, 2H); 2.72 (t, CH₂CONH, J=7.3 Hz, 2H); 2.5 (s, NHCOCH₃, 3H); 2.36 (t, CH₂SCH₂NHCOCH₃, J=7.23 Hz, 2H); 1.67-1.57 (m, CH₂ Lysine, 2H); 1.42-1.33 (m, CH₂ Lysine, 4H); ¹³C NMR (300 MHz, DMSO-d₆, 298 K) 174.48 (CO); 170.70 (CO); 169.68 (CO); 156.58 (CO); 144.25 (2 C); 141.15 (2 C); 128.85 (CH); 128.08 (2 CH); 127.51 (2 CH); 125.73 (2 CH); 66.03 (CH₂); 54.26 (CH); 47.10 (CH); 40.21 (CH₂); 38.73 (CH₂); 36.09 (CH₂); 30.91 (CH₂); 29.12 (CH₂); 26.64 (CH₂); 23.50 (CH₂); 23.24 (CH₃); MS (MALDI-TOF) Calculated for O₂₇H₃₃N₃O₆S: 527.21. Found [M+Na⁺]=550.11; [M+K⁺]=566.20.

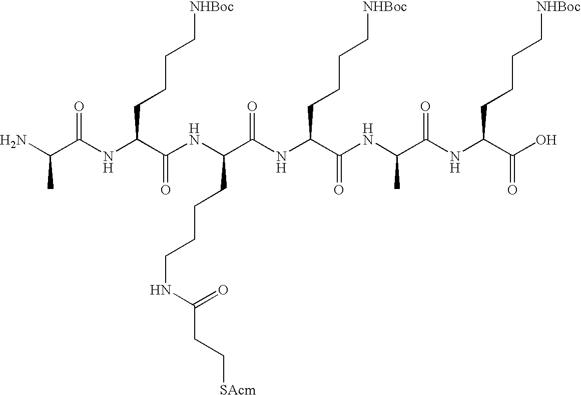

### H-(D)Ala-Lys(Boc)-(D)Lys(Acm)-Lys(Boc)-(D)Ala-Lys(Boc)-OH (IV.8)

800 mg of 2-chlorotrityl chloride resin (load=1.6 mmol/g) are washed twice with distilled CH₂Cl₂. To this resin, addition of a mixture of Fmoc-Lys(Boc)-OH (2 eq.) and DIEA (6 eq.) in CH₂Cl₂ is made. After stirring for 3 hours, the resin is filtered and washed with CH₂Cl₂. The resin is allowed to swell in methanol under stirring for 1 hour and then washed with DMF, isopropanol, CH₂Cl₂, diethyl ether and dried under vacuum. The load of the resin is determined by a UV reading of the Fmoc derivative following treatment with 20% piperidine in DMF (load=0.3 mmol/g).

FmocXaaOH (5 eq.) is coupled twice at room temperature for 30 min. to the deprotected Fmoc resin in presence of BOP (5 eq.), HOBt (1-hydroxybenzotriazole) (5 eq.) and DIEA (15 eq.). The compound IV.7 (2.5 eq.) is coupled once at room temperature for 2 hours in presence of BOP (2.5 eq.), HOBt (2.5 eq.) and DIEA (10 eq.). After repeating such steps of deprotection-coupling, the peptide is cleaved from the resin with a mixture of HFIP (hexafluoroisopropanol) and CH₂Cl₂ (60/40). After stirring for 2 hours, the resin is filtered and washed several times with CH₂Cl₂. Evaporation of the solvent provides compound IV.8 (yield=100%). HPLC t_{R} 7.62 min. (linear gradient, 30-100% B, 20 min); Purity of the crude product as determined by HPLC (linear gradient, 30-100% B, 20 min.): 73%; MS (MALDI-TOF) Calculated for C₅₁H₉₃N₁₁O₁₅S: 1131.66. Found: [M+Na⁺]=1154.27, [M+K⁺]=1170.21; HRMS (ESI): m/z: Calculated for C₅₁H₉₄N₁₁O₁₅S: 1132.66. Found: 1132.6646; Calculated for C₅₁H₉₃N₁₁O₁₅SNa: 1154.66. Found: 1154.6466.

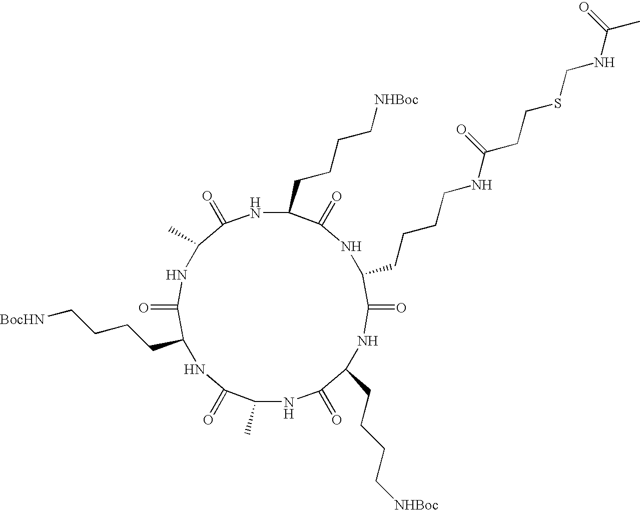

### Cyclo[(D)Ala-Lys(Boc)-(D)Lys(Acm)-Lys(Boc)-(D)Ala-Lys(Boc)] (IV.9)

The peptide IV.8 (500 mg 0.44 mmol) is dissolved into DMF at room temperature. EDC, HCl (101.16 mg; 0.53 mmol), HOBt (71.56 mg; 0.53 mmol) and DIEA (110 μL; 0.66 mmol) are added in succession. After stirring for 3 days at room temperature, the reaction mixture is precipitated in a big volume of saturated NaHCO₃. The precipitate is filtered and washed with saturated NaHCO₃ then with water, 1N KHSO₄, brine, ethyl acetate, acetonitrile and cyclohexane. The white solid IV.9 is dried under vacuum (222 mg, yield: 77%); purity of the crude product as determined by HPLC (linear gradient, 30-100% B, 20 min.): 87%; MS (MALDI-TOF) Calculated for C₅₁H₉₁N₁₁O₁₄S: 1113.65. Found: [M+Na⁺]=1136.44; [M+K⁺]=1152.42.

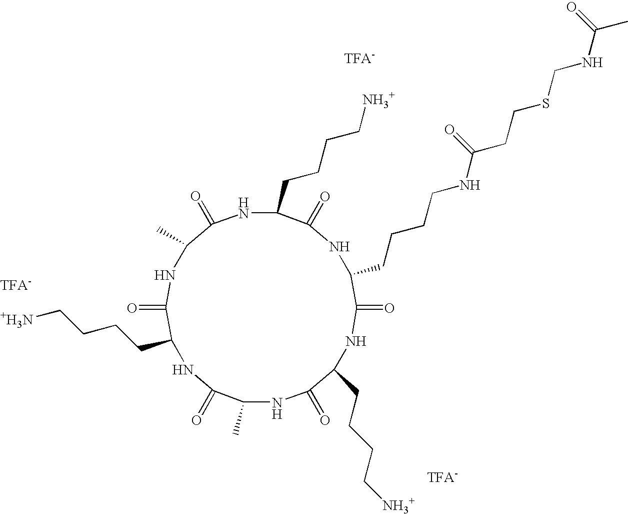

### cyclo[D)Ala-Lys-(D)Lys(Acm)-Lys-(D)Ala-Lys] (IV.10)

Compound IV.9 (100 mg; 0.089 mmol) is dissolved into trifluoroacetic acid (5 ml) with 5% water. After stirring for 20 min. at room temperature, the reaction mixture is precipitated with ether. The desired TFA salt is filtered and washed with ether. The white solid IV.10 is dried under vacuum (84 mg, yield: 85%). HPLC t_{R} 6.56 min. (linear gradient, 5-65% B, 20 min); purity of the crude product as determined by HPLC (linear gradient, 5-65% B, 20 min): 86%; MS (MALDI-TOF) Calculated for C₃₆H₆₇N₁₁O₈S: 813.05. Found: [M+Na⁺]=836.44; [M+K⁺]=852.41; HRMS (ESI): m/z: Calculated for C₃₆H₆₈N₁₁O₈S: 814.05. Found: 814.497.

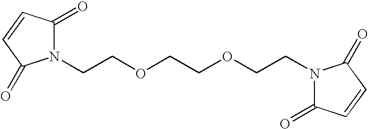

### Bismaleimidediethylene glycol (IV.11)

To a stirred solution of N,N′-diaminodiethylene glycol (100 mg; 0.67 mmol) in saturated NaHCO₃/THF (Vt=6 ml, 1:1 v/v) at 0° C., there is added N-(methoxy-carbonyl)maleimide (251 mg; 1.62 mmol). The reaction mixture is stirred at 0° C. for 10 minutes and is kept thereafter under stirring for 4 hours at room temperature. Next, the product is isolated by extraction with ethyl acetate. The pooled organic layers are washed water and brine, dried over Na₂SO₄, filtered and concentrate under vacuum to thereby obtain Compound IV.11 150 mg, yield=72%): HPLC t_{R} 10.40 min (linear gradient, 30-100% B, 20 min); ¹H NMR (300 MHz, CDCl₃, 298 K) δ6.67 (s, CH maleimide, 4H); 3.68-3.63 (m, NCH₂CH₂O, 4H); 3.58-3.54 (m, NCH₂CH₂O, 4H); 3.51 (s, OCH₂CH₂O, 4H); ¹³C NMR (300 MHz, CDCl₃, 298 K) 170.66 (4 CO); 134.13 (4 CH); 69.97 (2 CH₂); 67.77 (2 CH₂); 37.09 (2 CH₂); HRMS (ESI): m/z: Calculated for C₁₄H₁₇N₂O₆: 309.10. Found: 309.1081; Calculated for C₁₄H₁₆N₂O₆Na: 331.10. Found: 331.0901.

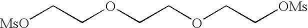

### Dimesylate (IV.12)

Triethylene glycol (2 g; 13.31 mmol) is dissolved into CH₂Cl₂ (20 ml). DIEA (5.6 ml; 33.3 mmol) and methanesulphonyl chloride (2.6 ml; 33.3 mmol) are added in succession. The mixture is stirred for 3 hours and CH₂Cl₂ is afterward evaporated. The residue is retaken up into ethyl acetate. The organic layer is washed with saturated NaHCO₃, water, 1N KHSO₄ and brine. Drying over Na₂SO₄ and evaporation of the solvent provide Compound IV.12 (3.93 g; yield=96%): ¹H NMR (300 MHz, CDCl₃, 298 K) δ 4.25-4.22 (m, CH₂OMs, 4H); 3.64 (m, CH₂CH₂OMs, 4H); 3.55 (s, OCH₂CH₂O, 4H); 2.96 (s, SO₂CH₃, 6H); ¹³C NMR (300 MHz, CDCl₃, 298 K) 70.33 (CH₂); 69.43 (CH₂); 68.82 (CH₂); 37.42 (CH₃).

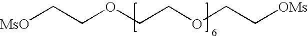

### Dimesylate (IV.14)

Octaethylene glycol (540 mg; 1.46 mmol) is dissolved into CH₂Cl₂ (2 ml). To this mixture, addition is made in succession at 0° C. of triethylamine (606 μL; 4.37 mmol) and methanesulphonyl chloride (288 μL; 3.6 mmol). After stirring for 4 hours, the solvent is evaporated. The residue is retaken into ethyl acetate and the organic layer is washed with saturated NaHCO₃ and 1N KHSO₄. Drying over Na₂SO₄ and evaporation of the solvent yield Compound IV.14 (560 mg; yield=73%): ¹H NMR (300 MHz, CDCl₃, 298 K) 4.26-4.22 (m, CH₂O Ms, 4H); 3.65-3.62 (m, CH₂CH₂O Ms, 4H); 3.53-3.50 (m, OCH₂CH₂O, 24H); 2.96 (s, OSO₂CH₃, 6H); ¹³C NMR (300 MHz, CDCl₃, 298 K) 70.43 (CH₂); 69.47 (CH₂); 68.88 (CH₂); 37.57 (CH).

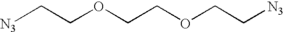

### 1,2-Bis(2-azidoethoxy)ethane (IV.15)

Sodium azide (1.33 g; 20.57 mmol) is added to a solution of Compound IV.12 (1.05 g; 3.4 mmol) into DMF (10 ml). The reaction mixture is heated overnight at 60° C. After cooling, water is added and the aqueous layer is extracted with ether. The organic layer is then washed with water and dried over Na₂SO₄. Evaporation of the solvent yields Compound IV.15 (670 mg, yield=96%): HPLC t_{R} 8.29 min (linear gradient, 30-100% B, 20 min); ¹H NMR (300 MHz, CDCl₃, 298 K) δ2.98-2.94 (m, CH₂CH₂N₃, 4H); 2.94 (s, OCH₂CH₂O, 4H); 2.67-2.63 (m, CH₂N₃, 4H); ¹³C NMR (300 MHz, CDCl₃, 298 K) 70.44 (CH₂); 69.88 (CH₂); 50.45 (CH₂).

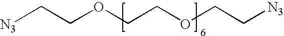

### Diazide (IV.17)

Sodium azide (170 mg; 2.6 mmol) is added to a stirred solution of Compound IV.14 (550 mg, 1.044 mmol) into DMF (8 ml). The reaction mixture is heated overnight at 60° C. After cooling, water is added and the aqueous layer is extracted with ether. The organic layer is then washed with water and dried over Na₂SO₄. Evaporation of the solvent yields Compound IV.17 (300 mg, yield=70%): HPLC t_{R} 6.59 min (linear gradient, 30-100% B, 20 min); ¹H NMR (300 MHz, CDCl₃, 298 K) δ3.66-3.60 (m, OCH₂CH₂O and CH₂CH₂N₃, 28H); 3.38-3.33 (m, CH₂N₃, 4H); ¹³C NMR (300 MHz, CDCl₃, 298 K) 70.68 (CH₂); 70.66 (CH₂); 70.62 (CH₂); 70.57 (CH₂); 50.67 (CH₂).

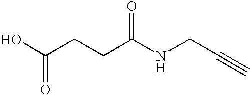

### N-Propyl-2-ynyl-succinamic Acid (Nps) (IV.18)

Dihydrofuran-2,5-dione (3.9 g; 40 mmol) is dissolved into acetonitrile (40 ml). Addition of prop-2-yn-1-amine (1.5 ml; 51 mmol) is made. After stirring for 2 hours, precipitation occurs. The precipitate is filtered and washed with acetonitrile. Compound IV.18 is obtained (4.12 g; yield=71%): ¹H NMR (300 MHz, DMSO-d₆, 298 K) δ3.83 (dd, NHCH₂CCH, J=5.4 and 2.5 Hz, 2H); 3.33 (d, CCH, J=2.5 Hz, 1H); 2.42-2.36 (m, CH₂CO₂H, 2H); 2.33-2.27 (m, CH₂CONH, 2H); ¹³C NMR (300 MHz, DMSO-d₆, 298 K) 174.33 (CO); 172.29 (CO); 81.69 (CH); 73.45 (C); 30.35 (CH₂); 29.66 (CH₂); 28.24 (CH₂).

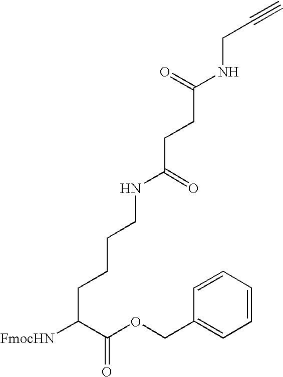

### (9H-Fluoren-9-yl)methyl 1-((benzyloxy)carbonyl)-5-(succinamido)pentylcarbamate (Fmoc-Lys(Nps)-OBn) (IV.19)

Compound IV.4 (9.7 mmol) is dissolved into acetonitrile (30 ml) along with DIEA (1.9 ml; 11.6 mmol). 3-(prop-2-ynylcarbamoyl)propanoic acid (1.8 g; 11.6 mmol), BOP (5.13 g; 11.6 mmol) and DIEA (1.9 ml; 11.6 mmol) are added in succession. After stirring for 3 hours, precipitation occurs. The precipitate is filtered and washed with saturated NaHCO₃, water and 1N KHSO₄ followed by washing with acetonitrile and diisopropyl ether. There is obtained Compound IV.9 (3.7 g; yield=64%): ¹H NMR (300 MHz, DMSO-d₆, 298 K) δ8.32 (t, NH, J=5.27 Hz, 1H); 7.90-7.85 (m, 2 NH and CH Arom Fmoc, 4H); 7.71 (d, CH Arom Fmoc, J=7.33 Hz); 7.42 (t, CH Arom Fmoc, J=7.32 Hz, 2H); 7.34-7.28 (m, CH Arom Fmoc and CH Arom Bn, 7H); 5.13 (s, CH₂Ph, 2H); 4.32-4.10 (m, CH₂ Fmoc and CH Fmoc, 3H); 4.09-4.02 (dd, NHCHCH₂, 1H); 3.84 (dd, NHCH₂CCH, J=5.25 and 2.32 Hz, 2H); 3.085 (t, CCH, J=2.35 Hz, 1H); 2.35-2.30 (m, COCH₂CH₂CO, 4H); 1.78-1.59 (m, CH₂ Lysine, 2H); 1.41-1.22 (m, CH₂ Lysine, 4H); ¹³C NMR (300 MHz, DMSO-d₆, 298 K) 172.8 (CO); 171.62 (CO); 171.46 (CO); 155.66 (CO); 144.27 (2 C); 141.18 (C); 136.42 (2 C); 128.86 (2 CH); 128.47 (2 CH); 128.21 (2 CH); 128.11 (CH); 127.53 (2 CH); 125.7 (2 CH); 120.57 (2 CH); 81.73 (CH); 73.32 (C); 66.34 (CH₂); 66.16 (CH₂); 54.44 (CH); 47.10 (CH); 38.67 (CH₂); 31.03 (CH₂); 30.76 (CH₂); 30.98 (CH₂); 29.10 (CH₂); 28.26 (CH₂); 23.38 (CH₂); MS (MALDI-TOF) Calculated for C₃₅H₃₇N₃O₆: 595.27. Found [M+H⁺]=595.78; [M+Na⁺]=618.26; [M+K⁺]=634.35.

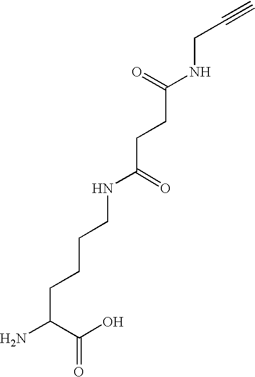

### Acid 2-amino-6-(succinamido)hexanoic (H-Lys(Nps)-OH) (IV.20)

Compound IV.9 (3.6 g; 6.0 mmol) is dissolved into DMF (25 ml). A solution of LiOH 1N (12 ml) is gently added at 0° C. After stirring for 4 hours, DMF is evaporated under reduced pressure and the residue is precipitated in acetonitrile to yield Compound IV.20 (912 mg; yield=54%).

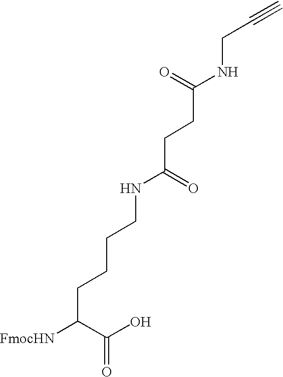

### Fmoc-D-Lys(Nps)-OH (IV.21)

Compound IV.20 (972 mg; 3.43 mmol) is dissolved into a mixture of water and acetone (50 ml, v/v: 1/1). K₂CO₃ (948 mg; 6.86 mmol) in a water solution is added (25 ml). Next, Fmoc-OSu (9-fluorenylmethyloxycarbonyl-N-hydroxysuccinimide) (1.16 g; 3.43 mmol) in acetone (25 ml) is gently added. After stirring overnight, water is added. The aqueous phase is quickly washed with ether and is adjusted to pH=3 by means of 1 N HCl and is subsequently extracted with CH₂Cl₂. Drying over Na₂SO₄ and evaporation of the filtrate provide an oil which is precipitated in CH₂Cl₂/IPE to thereby yield the pure Compound IV.21 (1.7 g; yield=62%): HPLC t_{R} 8.61 min (linear gradient, 30-100% B, 20 min); ¹H NMR (300 MHz, DMSO-d₆, 298 K) δ8.26 (t, NH, J=5.36 Hz, 1H); 7.89 (d, CH Arom Fmoc, J=7.42 Hz, 2H); 7.81 (t, NH, J=5.25 Hz, 1H); 7.73 (d, CH Arom Fmoc, J=7.38 Hz, 2H); 7.62 (d, FmocNH, J=7.98 Hz, 1H); 7.42 (t, CH Arom Fmoc, J=7.41 and 1.06 Hz, 2H); 7.33 (dt, CH Arom Fmoc, J=7.47 and 0.88 Hz, 2H); 4.29-4.19 (m, CH₂ Fmoc and CH Fmoc, 3H); 3.94-3.87 (m, FmocNHCH, 1H); 3.83 (dd, NHCH₂CCH, J=5.45 and 2.51 Hz, 2H); 3.07 (t, CCH, J=2.51 Hz, 1H); 3.04-2.96 (m, CH₂ Lysine, 2H); 2.33-2.26 (m, CH₂CONH and CH₂CONH, 4H); 1.72-1.54 (m, CH₂ Lysine, 4H); 1.41-1.24 (m, CH₂ Lysine, 4H); ¹³C NMR (300 MHz, DMSO-d₆, 298 K) 173.89 (CO); 171.04 (CO); 170.88 (CO); 156.08 (CO); 143.72 (2 C); 140.62 (2 C); 127.56 (CH); 126.98 (CH); 125.19 (CH); 120.03 (CH); 81.54 (CH); 72.80 (CH); 65.52 (CH₂); 53.68 (CH); 46.57 (CH); 38.73 (CH₂); 30.46 (CH₂); 30.41 (CH₂); 30.34 (CH₂); 28.60 (CH₂); 27.71 (CH₂); 22.99 (CH₂); MS (MALDI-TOF) Calculated for C₂₈H₃₁N₃O₆: 505.22. Found [M+Na⁺]=528.79; [M+K⁺]=545.57.

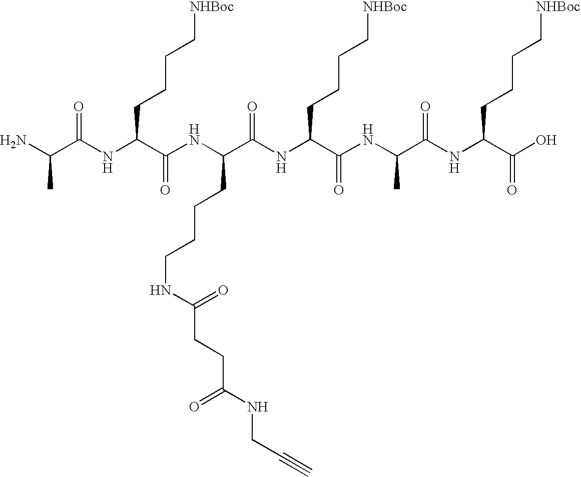

### H-(D)Ala-Lys(Boc)-(D)Lys(Nps)-Lys(Boc)-(D)Ala-Lys(Boc)-OH (IV.22)

The 2-chlorotrityl chloride resin (load=1.8 mmol/g) is washed twice with distilled CH₂Cl₂. To the resin, a mixture of Fmoc-Lys(Boc)-OH (2 eq.) and DIEA (6 eq.) in CH₂Cl₂ is added. After stirring for 3 hours, the resin is filtered and washed with CH₂Cl₂. The resin is allowed to swell back in methanol under stirring for 1 hour and is next washed with DMF, isopropanol, CH₂Cl₂, diethyl ether and dried under vacuum. The resin load is determined by a UV reading of the Fmoc derivative following treatment with 20% piperidine in DMF (load=0.5 mmol/g).

FmocXaaOH (5 eq.) is coupled twice at room temperature for 30 min. to the deprotected Fmoc resin in the presence of BOP (5 eq.), HOBt (5 eq.) and DIEA (15 eq.). The compound IV.21 (2.5 eq.) is coupled once at room temperature for 2 hours to the deprotected Fmoc resin in the presence of BOP (2.5 eq.), HOBt (2.5 eq.) and DIEA (10 eq.). After repeating such deprotection and coupling steps, the peptide is cleaved from the resin with a mixture of HFIP and CH₂Cl₂ (60/40). After stirring for 2 hours, the resin is filtered and washed several times with CH₂Cl₂. Evaporation of the solvent yields peptide IV.22. (yield: 100%): HPLC t_{R} 8.54 min (linear gradient, 30-100% B, 20 min); Purity of the crude product as determined by HPLC (linear gradient, 30-100% B, 20 min): 60%; MS (MALDI-TOF) Calculated for C₅₂H₉₁N₁₁O₁₅: 1109.67. Found: [M+Na⁺]1132.45; [M+K⁺]=1148.43; HRMS (ESI): m/z: Calculated for C₅₂H₉₂N₁₁O₁₅: 1110.67. Found: 1110.6769; Calculated for C₅₂H₉₁N₁₁O₁₅Na: 1132.67. Found: 1132.6588.

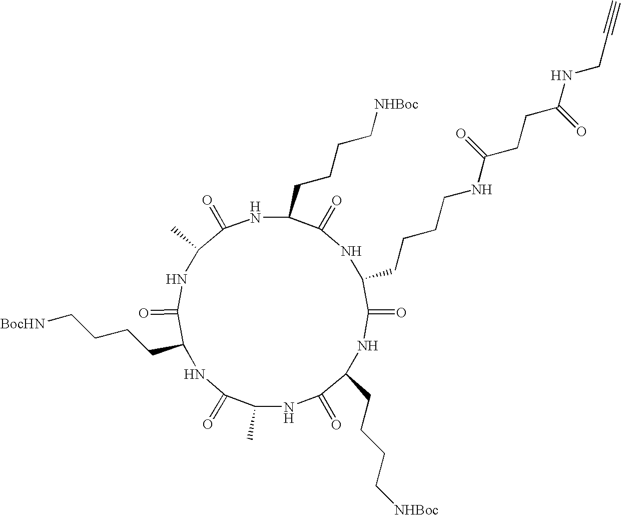

### Cyclo[(D)Ala-Lys(Boc)-(D)Lys(Nps)-Lys(Boc)-(D)Ala-Lys(Boc)] (IV.23)

Peptide IV.22 (60 mg; 0.054 mmol) is dissolved into DMF (12 ml) at room temperature. BOP (28.6 mg; 0.064 mmol) and DIEA (14 μL; 0.081 mmol) are added in succession After stirring for 3 days at room temperature, the reaction mixture is precipitated in a big volume of saturated NaHCO₃. The precipitate is filtered and washed with saturated NaHCO₃, followed by washing with water, 1N KHSO₄, brine, ethyl acetate, acetonitrile and cyclohexane. The white solid IV.23 is dried under vacuum (49 mg, yield: 83%) HPLC t_{R} 12.92 min. (linear gradient, 30-100% B, 20 min.); purity of the crude product as determined by HPLC (linear gradient, 30-100% B, 20 min): 60%; MS (MALDI-TOF) Calculated for C₅₂H₈₉N₁₁O₁₄: 1091.66. Found: [M+Na⁺]=1114.58; [M+K⁺]=1130.56.

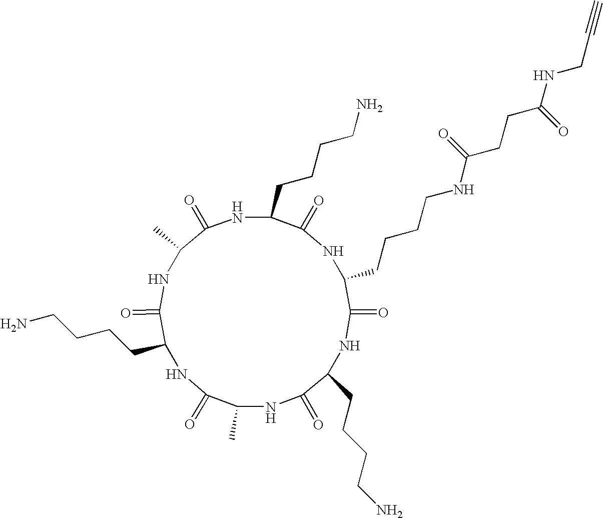

### cyclo[(D)Ala-Lys-(D)Lys(Nps)-Lys-(D)Ala-Lys] (IV.24)

Compound IV.23 (38 mg; 0.035 mmol) is dissolved into trifluoroacetic acid (3.5 ml) containing 5% water. After stirring for 15 min. at room temperature, the reaction mixture is precipitated in ether. The expected TFA salt is filtered, washed with ether. The white solid IV.23 is dried under vacuum (34 mg, yield: 87%): HPLC t_{R} 6.28 min. (linear gradient, 5-65% B, 20 min); purity of the crude product as determined by HPLC (linear gradient, 5-65% B, 20 min.): 66%: MS (MALDI-TOF) Calculated for C₃₇H₆₅N₁₁O₈: 791.5. Found: [M+H⁺]=792.57; [M+Na⁺]=814.54; [M+K⁺]=830.52.

### <<Click Chemistry>> General Procedure (IV.25-IV.27)

Compound IV.23 (1 eq) is dissolved into DMF (c=6.10⁻³ M). A 1N diazide solution in DMF (1 eq) is added. This mixture is purged by argon for 30 min. to flush oxygen. DIEA (25 eq), 2,6-lutidine (25 eq) and CuI (2 eq) are added in succession. After stirring for 3 days at room temperature, the reaction mixture is precipitated in a big volume of saturated NaHCO₃. The precipitate is filtered and washed with saturated NaHCO₃, followed by washing with water, 1N KHSO₄ and brine. The white solid is dried under vacuum. This compound is dissolved into trifluoroacetic acid containing 5% water. After stirring for 30 min. at room temperature, the reaction mixture is precipitated in ether. The desired TFA salt is filtered, washed with ether and dried under vacuum. Purification by semi-preparative C₁₈ RP-HPLC (5-65% B, 20 min) followed by lyophilization provide the expected compound.

IV.25: Using this general procedure, there is obtained Compound IV.25. The purity of the crude product is 47% (as determined by C₁₈ RP-HPLC). Purification by semi-preparative C₁₈ RP-HPLC yields the pure Compound IV.25 (yield 40% and purity>99%): HPLC t_{R} 6.63 min. (linear gradient, 5-65% B, 20 min.); MS (MALDI-TOF) Calculated for C₈₀H₁₄₂N₂₈O₁₈: 1783.11. Found: [M+Na⁺]=1806.48; [M+K⁺]=1823.39; HRMS (ESI): m/z: Calculated for C₈₀H₁₄₂N₂₈O₁₈Na: 1806.11. Found: 1806.095.

IV.26: Using this general procedure, there is obtained Compound IV.26. The purity of the crude product is 30% (as determined by C₁₈ RP-HPLC). Purification by semi-preparative C₁₈ RP-HPLC yields the pure Compound IV.26 (yield 13% and purity>99%):HPLC t_{R} 6.95 min. (linear gradient, 5-65% B, 20 min.); MS (MALDI-TOF) Calculated for C₈₂H₁₄₆N₂₈O₁₉. 1827.13. Found: [M+H⁺]=1828.14; [M+Na⁺]=1850.14; [M+K⁺]=1866.14.

IV.27: Using this general procedure, there is obtained Compound IV.27. The purity of the crude product is 54% (as determined by C₁₈ RP-HPLC). Purification by semi-preparative C₁₈ RP-HPLC yields the pure Compound IV.27 (yield 20% and purity>99%): HPLC t_{R} 7.8 min (linear gradient, 5-65% B, 20 min); MS (MALDI-TOF) Calculated for C₉₀H₁₆₂N₂₈O₂₃: 2003.24. Found: [M+H⁺]=2004.34; [M+Na⁺]=2026.34; [M+K⁺]=2042.31.

General Coupling Procedure

The core peptide IV.24, IV.25 or IV.26 (1 eq.) is dissolved into DMF. Addition is made at room temperature of the peptide (6.6 eq.), BOP (6.6 eq.) and DIEA (20 eq.). The reaction mixture is stirred for one week and precipitated in a big volume of saturated NaHCO₃. The precipitate is filtered and washed with saturated NaHCO₃, followed by washing with water, 1N KHSO₄, brine and cyclohexane. The solid is dried under vacuum.

The crude product is dissolved into trifluoroacetic acid containing 5% water. After stirring for 30 min. at room temperature, the reaction mixture is precipitated in ether. The desired TFA salt is filtered, washed with ether and dried under vacuum. Purification by semi-preparative C₁₈ RP-HPLC (5-65% B, 20 min) followed by lyophilization provides the desired ligand.

L41: Using the general procedure outlined above, there is obtained ligand L41. Purity of the crude ligand is 51% (as determined by C₁₈ RP-HPLC). Purification by semi-preparative C₁₈ RP-HPLC provides the pure L41 ligand (yield 12% and purity>99%): HPLC t_{R} 10.65 min (linear gradient, 5-65% B, 20 min); MS (MALDI-TOF) Calculated for C₂₇₂H₄₀₆N₆₄O₆₀. 5529.07. Found: [M+H⁺]=5529.78.

L42: Using the general procedure outlined above, there is obtained ligand

L42. Purity of the crude ligand is 58% (as determined by C₁₈ RP-HPLC). Purification by semi-preparative C₁₈ RP-HPLC provides the pure L42 ligand (yield 15% and purity>99%): HPLC t_{R} 10.89 min (linear gradient, 5-65% B, 20 min); MS (MALDI-TOF) Calculated for C₂₇₅H₄₁₁N₆₅O₆₁: 5600.11. Found: [M+H⁺]=5580.

L43: Using the general procedure outlined above, there is obtained ligand

L43. Purity of the crude ligand is 54% (as determined by C₁₈ RP-HPLC).

Purification by semi-preparative C18 RP-HPLC provides the pure L43 ligand (yield 15% and purity>99%): HPLC t_{R} 11.04 min (linear gradient, 5-65% B, 20 min); MS (MALDI-TOF) Calculated for C₂₈₂H₄₂₆ N₆₄O₆₅: 5749.2. Found: [M+H⁺]=5751.

1-16. (canceled)

17. A compound having the following formula (I): 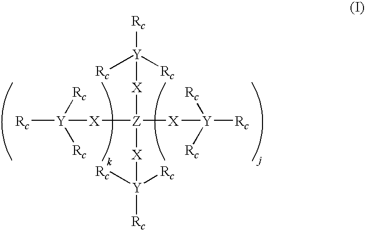
wherein:
k and j represent independently from each other 0 or 1,
Y represents a macrocycle the ring of which includes from 9 to 36 atoms, and is functionally substituted by three amine functionalities and by a carbon chain allowing binding of a Z spacer via an X bond,
R_{c} represents a binding motif to a receptor belonging to the TNF superfamily, and preferably represent a ligand derived-sequence selected among the residues interfacing with the ligand receptor, which sequence may interact with the receptor, said ligand being selected among receptor ligands belonging to the TNF superfamily, namely among the following ligands: EDA, CD40L, FasL, OX40L, AITRL, CD30L, VEGI, LIGHT, 4-1 BBL, CD27L, LTα, TNF, LTβ, TWEAK, APRIL, BLYS, RANKL and TRAIL,
X represents a chemical functionality which allows the Y group to be linked to the spacer and is selected among the following functional groups: 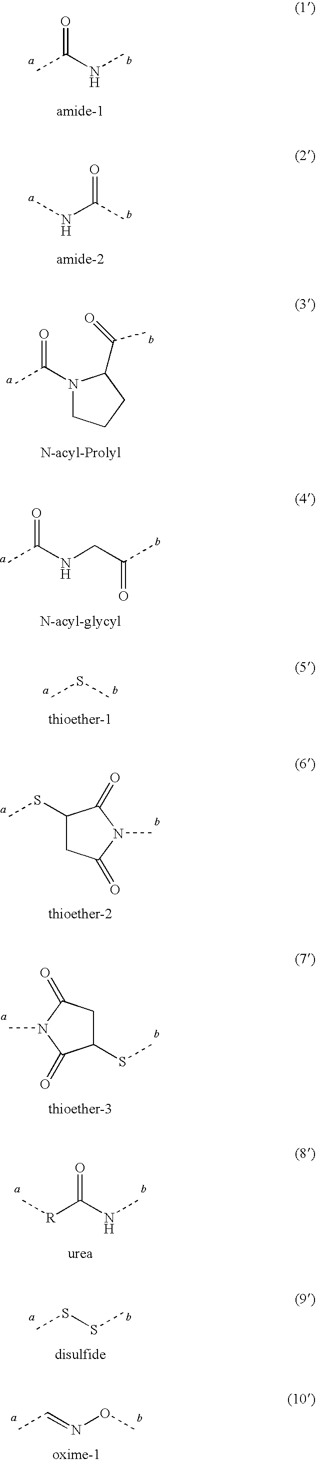 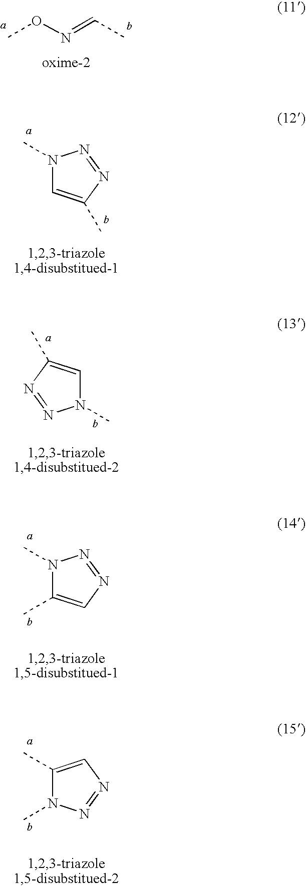
a designating the bond to the Y group and b designating the bond to the Z group,
Z represents a bi, tri- or tetrafunctional spacer having one of the following formulae:
if j=k=0:
if X represents a group of formulae (1′), (8′), (9′), (5′), (6′), (7′), (13′) and (15′), Z has one of the following formulae: 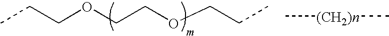
m being an integer ranging from 1 to 40,
n being an integer ranging from 1 to 10,
if X represents a group of formulae (2′), (3′) and (4′), Z has one of the following formulae: 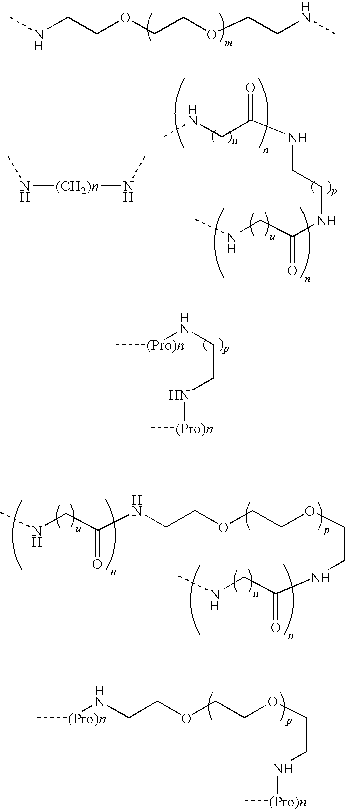
if X represents a group of formulae (12′) and (14′), Z has one of the following formulae: 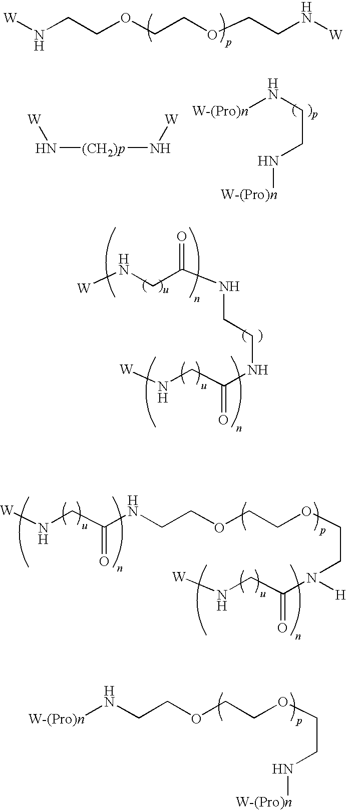
m and n being as defined above,
p being an integer ranging from 1 to 6,
u being an integer ranging from 1 to 4,
W designating a group of formula 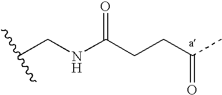
or a group of formula 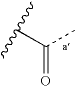
the W group being bound to the NH group through the dotted line bond a′
if X represents a group of formulae (1′), (2′), (8′), (9′), (5′), (6′), (7′), (10′), (11′), (13′) and (15′), Z has one of the following formulae: 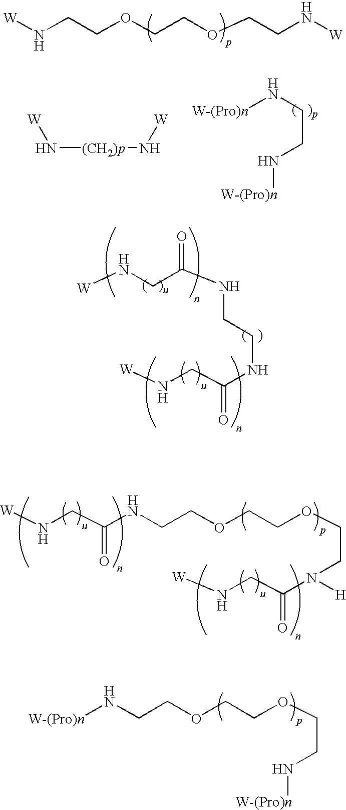
m and n being as defined above,
p being an integer ranging from 1 to 6,
u being an integer ranging from 1 to 4,
W designating a group of formula 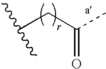
r being an integer ranging from 1 to 4,
the W group being bound to the NH group via the dotted line bond a′
if j=1 or k=1
if X represents a group of formulae (12′) and (14′), Z has one of the following formulae: 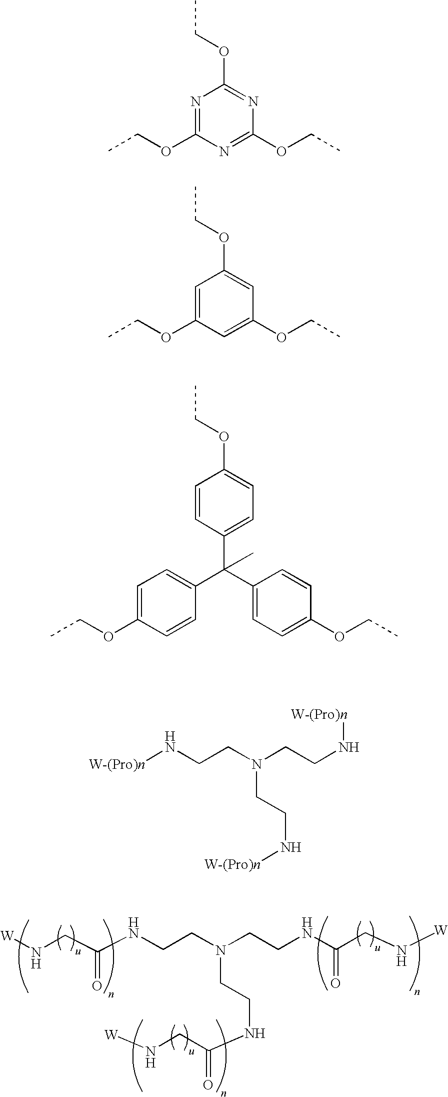
u being an integer ranging from 1 to 4,
n being an integer ranging from 1 to 10,
W designating a group of formula 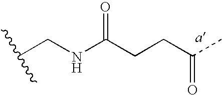
or a group of formula 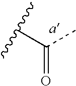
the W group being bound to the NH group through the dotted line bond a′,
if X represents a group of formulae (1′), (2′), (8′), (9′), (5′), (6′), (7′), (10′), (11′), (13′) and (15′), Z has one of the following formulae: 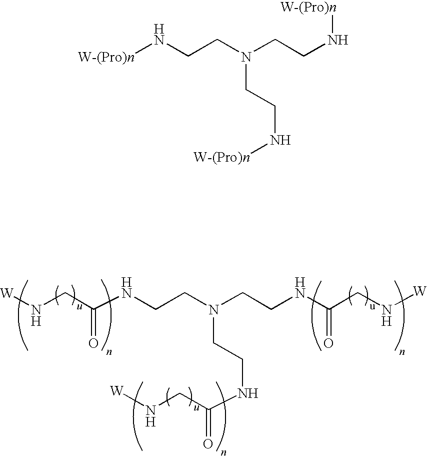
u being an integer ranging from 1 to 4,
n being an integer ranging from 1 to 10,
W designating a group of formula 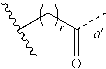
r being an integer ranging from 1 to 4,
the W group being bound to the NH group via the dotted line bond a′
Z can also represent one of the following formulae: 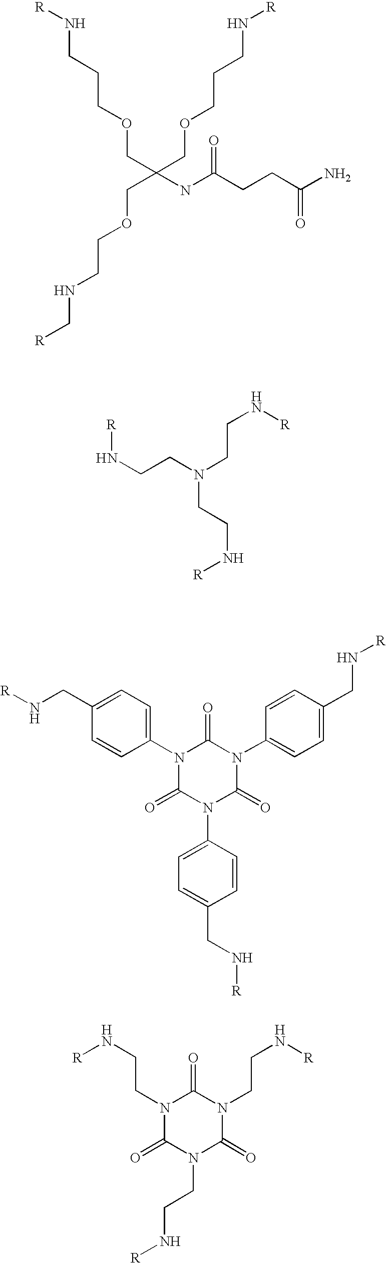 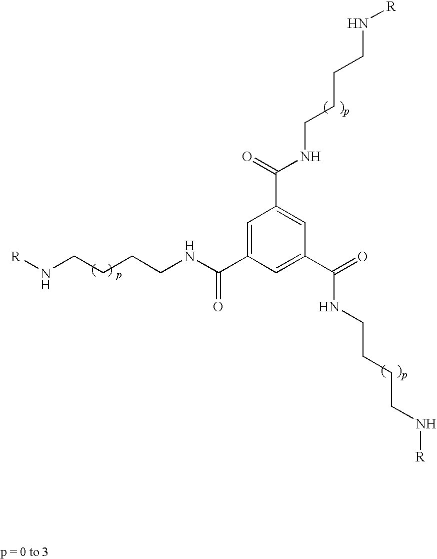
wherein:
if X represents a group of formulae (1′), (8′), (9′), (5′), (6′), (7′), (13′) and (15′):
R represents one of the following groups: 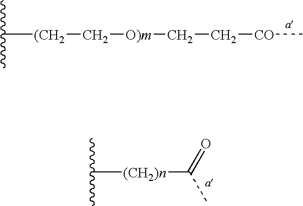
m ranging from 3 to 6 n ranging from 1 to 10 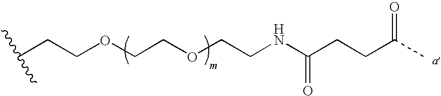
m ranging from 1 to 40
the R group being bound to the NH group via the dotted line bond a′,
if X represents a group of formulae (2′), (3′) and (4′):
R represents one of the following groups: 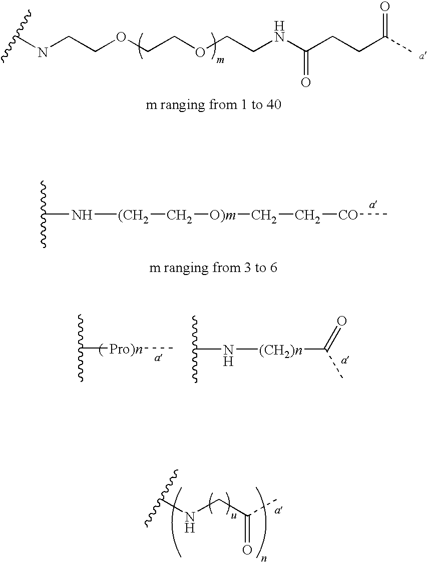
n being an integer ranging from 1 to 10,
u being an integer ranging from 1 to 4,
the R group being bound to the NH group through the dotted line bond a′,
if X represents a group of formulae (12′) and (14′):
R represents one of the following groups: 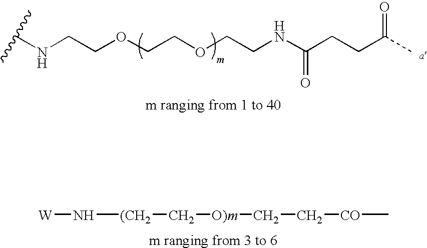
the R group being bound to the NH group through the dotted line bond a′, 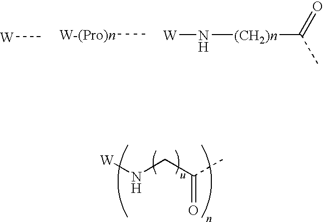
n being an integer ranging from 1 to 10,
W designating a group of formula 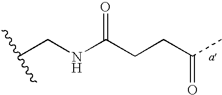
or a group of formula 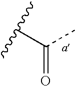
the W group being bound to the NH group through the dotted line bond a′
if X represents a group of formulae (1′), (2′), (8′), (9′), (5′), (6′), (7′), (10′), (11′), (13′) and (15′):
R represents one of the following groups: 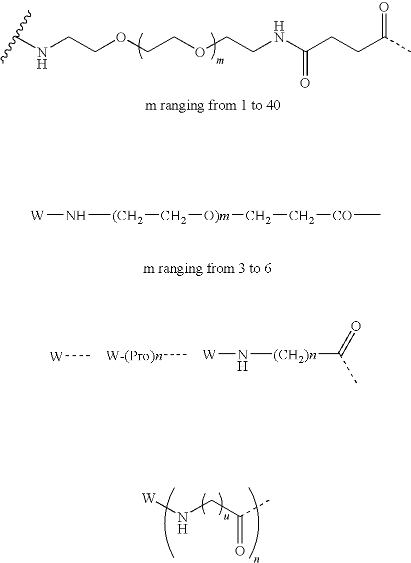
u and n being as specified above,
W designating a group of formula 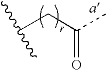
r being an integer ranging from 1 to 4,
the W group being bound to the NH group through the dotted line bond a′.

18. A compound in accordance with claim 17, characterized in that R_{c} represents a human or murine CD40 receptor ligand (CD40L)-derived peptide, with said peptide belonging to the primary sequence of the CD40L ligand of CD40 the amino acid number of which is included between 3 and 10.

19. A compound in accordance with claim 17, characterized in that R_{c} represents a human or murine CD40 receptor ligand (CD40L)-derived peptide, with said peptide belonging to the primary sequence of the CD40L ligand the amino acid number of which is included between 3 and 10 selected among the following sequences: LQWAEKGYYTMSNN (SEQ ID NO: 1); LQWAKKGYYTMKSN (SEQ ID NO: 2); PGRFERILLRAANTH (SEQ ID NO: 3); SIGSERILLKAANTH (SEQ ID NO: 4).

20. A compound in accordance with claim 17, characterized in that R_{c} represents a group of formula H—Xₐ—(X_{b})ₗ—X_{c}—X_{d}—Xₑ—(X_{f})ᵢ— or H—X′ₐ-L-X′_{b}—X_{c}—X_{d}—Xₑ—(X_{f})ᵢ—; wherein
l represents 0 or 1,
i represents 0 or 1,
Xₐ is selected among the following amino acid residues:
lysine;
arginine;
ornithine;
β-amino acids corresponding to lysine, arginine or ornithine bearing the substitution in positions α or β;
tranexamic acid;
N-methyl-tranexamic acid;
8-amino-3,6-dioxaoctanoic acid;
4(piperidin-4-yl)butanoic acid;
3(piperidin-4-yl)propionic acid;
N-(4-aminobutyl)-glycine;
NH₂—(CH₂)ₙ—COON, n ranging from 1 to 10;
NH₂—(CH₂—CH₂—O)ₘ—CH₂CH₂COOH, m ranging from 3 to 6;
4-carboxymethyl-piperazine;
4-(4-aminophenyl)butanoic acid;
3-(4-aminophenyl)propanoic acid;
4-aminophenylacetic acid;
4-(2-aminoethyl)-1-carboxymethyl-piperazine;
trans-4-aminocyclohexanecarboxylic acid;
cis-4-aminocyclohexanecarboxylic acid;
cis-4-aminocyclohexane acetic acid;
trans-4-aminocyclohexane acetic acid;
4-amino-1-carboxymethyl piperidine;
4-aminobenzoic acid;
4(2-aminoethoxy)benzoic acid;
X_{b} is selected among the following amino acid residues:
glycine;
isoleucine;
leucine;
valine;
asparagine;
D-alanine;
D-valine;
L-proline optionally substituted in position β, γ or δ;
D-proline optionally substituted in position β, γ or δ;
N-alkyl-natural amino acids, the alkyl group being a methyl, ethyl or benzyl group;
dialkyl-acyclic amino acids of the following formula: 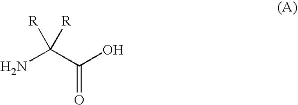
R designating H, Me, Et, Pr or Bu;
dialkyl-cyclic amino acids of the following formula: 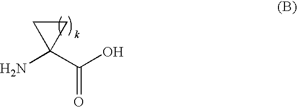
k designating 1, 2, 3 or 4;
X_{c} is selected among the following amino acid residues:
tyrosine;
isoleucine;
leucine;
valine;
phenylalanine;
3-nitro-tyrosine;
4-hydroxymethyl-phenylalanine;
3,5-dihydroxy-phenylalanine;
2,6-dimethyl-tyrosine;
3,4-dihydroxy-phenylalanine (DOPA);
X_{d} is selected among the following amino acid residues:
tyrosine;
isoleucine;
leucine;
valine;
phenylalanine;
3-nitro-tyrosine;
4-hydroxymethyl-phenylalanine;
3,5-dihydroxy-phenylalanine;
2,6-dimethyl-tyrosine;
3,4-dihydroxy-phenylalanine;
Xe is selected among the following amino acid residues:
arginine;
-(Gly)ₙ-, n ranging from 1 to 10;
-(Pro)ₙ-, n ranging from 1 to 10;
NH₂—(CH₂)ₙ—COON, n ranging from 1 to 10;
NH₂—(CH₂—CH₂—O)ₘ—CH₂CH₂COOH, m ranging from 3 to 6;
8-amino-3,6-dioxaoctanoic acid;
tranexamic acid;
N-methyl-tranexamic acid;
4(piperidin-4-yl)butanoic acid;
3(piperidin-4-yl)propionic acid;
N-(4-aminobutyl)-glycine;
4-carboxymethyl-piperazine;
4-(4-aminophenyl)butanoic acid;
3-(4-aminophenyl)propanoic acid;
4-aminophenylacetic acid;
4-(2-aminoethyl)-1-carboxymethyl-piperazine;
trans-4-aminocyclohexanecarboxylic acid;
cis-4-aminocyclohexanecarboxylic acid;
cis-4-aminocyclohexane acetic acid;
trans-4-aminocyclohexane acetic acid;
4-amino-1-carboxymethyl piperidine;
4-aminobenzoic acid;
4(2-aminoethoxy)benzoic acid;
X_{f} is selected among the following amino acid residues:
-(Gly)ₙ-, n ranging from 1 to 10;
-(Pro)ₙ-, n ranging from 1 to 10;
NH₂—(CH₂)ₙ—COON, n ranging from 1 to 10;
NH₂—(CH₂—CH₂—O)ₘ—CH₂CH₂COOH, m ranging from 3 to 6;
8-amino-3,6-dioxaoctanoic acid;
tranexamic acid;
N-methyl-tranexamic acid;
4(piperidin-4-yl)butanoic acid;
3(piperidin-4-yl)propionic acid;
N-(4-aminobutyl)-glycine;
4-carboxymethyl-piperazine;
4-(4-aminophenyl)butanoic acid;
3-(4-aminophenyl)propanoic acid;
4-aminophenylacetic acid;
4-(2-aminoethyl)-1-carboxymethyl-piperazine;
trans-4-aminocyclohexanecarboxylic acid;
cis-4-aminocyclohexanecarboxylic acid;
cis-4-aminocyclohexane acetic acid;
trans-4-aminocyclohexane acetic acid;
4-amino-1-carboxymethyl piperidine;
4-aminobenzoic acid;
4(2-aminoethoxy)benzoic acid;
-L- represents a peptide-like type bond between residues X′ₐ and X′_{b}
if X′_{b} represents a proline side chain,
Fₖ and Fₖ′ represent, independently from each other, a hydrogen, a halogen, an alkyl group of 1 to 20 carbon atoms, or an aryl group the ring structure of which includes from 5 to 20 carbon atoms,
X′ₐ designates the side chain of lysine, arginine or ornithine; and
X′_{b} designates the side chain of one of the following amino acid residues:
glycine;
isoleucine;
leucine;
valine;
asparagine;
D-alanine;
D-valine;
L-proline optionally substituted in position β, γ or δ;
D-proline optionally substituted in position β, γ or δ;
N-alkyl natural amino acids, the alkyl group of which is a methyl, ethyl or benzyl group;
dialkyl-acyclic amino acids of the following formula: 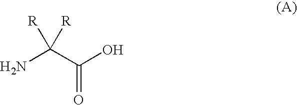
R designating H, Me, Et, Pr or Bu;
dialkyl-cyclic amino acids of the following formula: 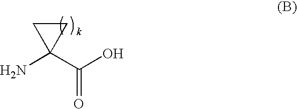
k designating 1, 2, 3 or 4.

21. A compound according to claim 20, wherein -L- represents a peptide-like type bond between residues X′ₐ and X′_{b} selected from the list given below:
-L-=-ψ(CH₂CH₂)—; -ψ(CH(Fₖ)═CH(Fₖ′))-; -ψ(CH₂NH)—; -ψ(NHCO)—;
-ψ(NHCONH)—; -ψ(CO—O)—; -ψ(CS—NH)—; -ψ(CH(OH)—CH(OH))-; -ψ(S—CH₂)—;
-ψ(CH₂—S)—; -ψ(CH(CN)—CH₂)—; -ψ(CH(OH))-; -ψ(COCH₂)—; -ψ(CH(OH)CH₂)—; -ψ(CH(OH)CH₂NH)—; -ψ(CH₂)—; -ψ(CH(Fₖ))-; -ψ(CH₂O)—; -ψ(CH₂—NHCONH)—; -ψ(CH(Fₖ)NHCONFₖ′)—; -ψ(CH₂—CONH)—;
-ψ(CH(Fₖ)CONH)—; -ψ(CH(Fₖ)CH(Fₖ′)CONH)—;
or -ψ(CH₂N)—; -ψ(NHCON)—; -ψ(CS—N)—; -ψ(CH(OH)CH₂N)—;
-ψ(CH₂—NHCON)—; -ψ(CH₂—CON)—; -ψ(CH(Fₖ)CON)—;
-ψ(CH(Fₖ)CH(Fₖ′)CON)—
if X′_{b} represents a proline side chain,
Fₖ and Fₖ′ represent, independently from each other, a hydrogen, a halogen, an alkyl group of 1 to 20 carbon atoms, or an aryl group the ring structure of which includes from 5 to 20 carbon atoms,

22. A compound in accordance with claim 17, characterized in that Y has the following formula (II): 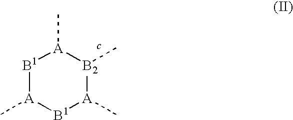
wherein:
A designates an amino acid residue or an amino acid derivative randomly selected among: 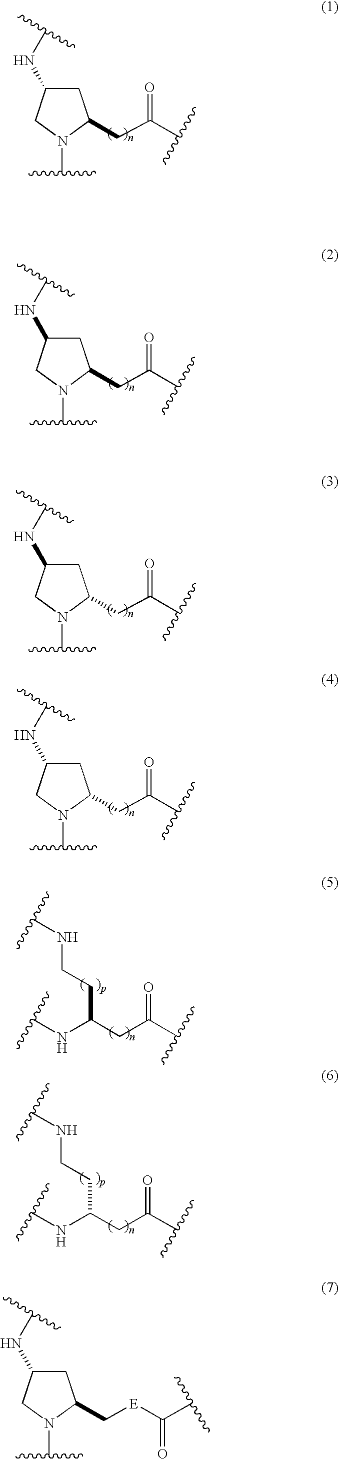 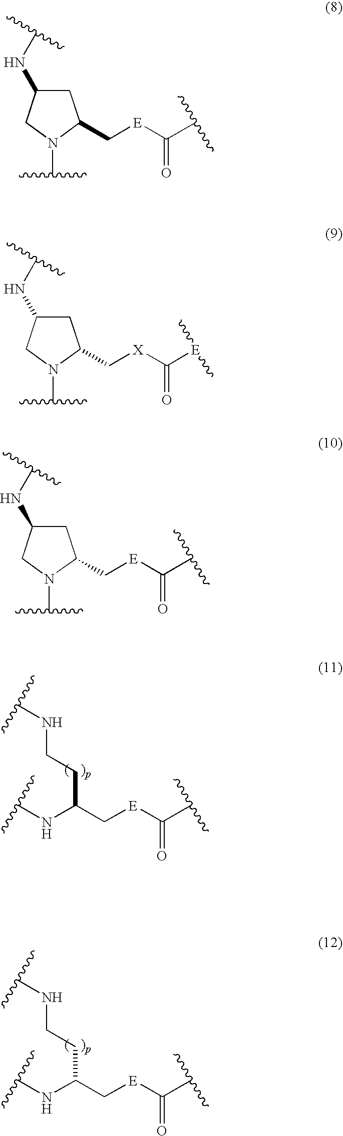
n designating 0, 1, 2 or 3;
p designating 0, 1, 2 or 3;
E designating NH or 0;
B¹ designates an amino acid residue or an amino acid derivative independently selected among: 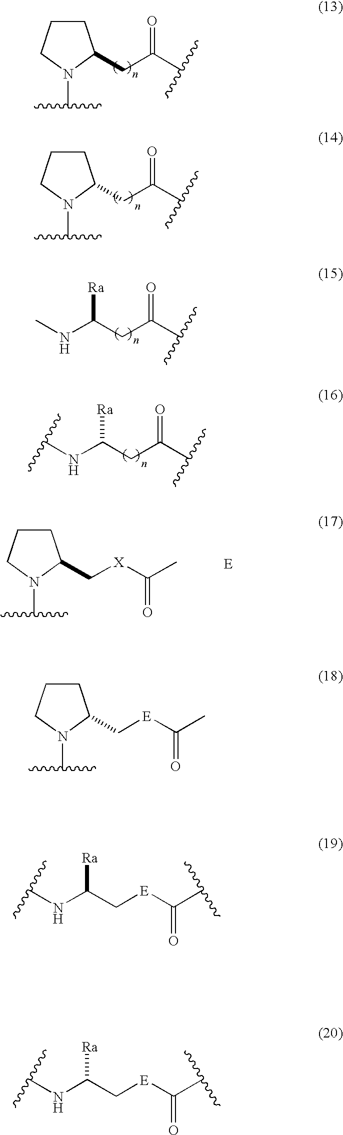
n designating 0, 1, 2 or 3;
p designating 0, 1, 2 or 3;
E designating NH or O;
Rₐ represents the carbon chain of a C₁₋₈ alkylated proteinogenic amino acid,
B² is independently selected among the following groups:
the c bond designating the bond to the X group, 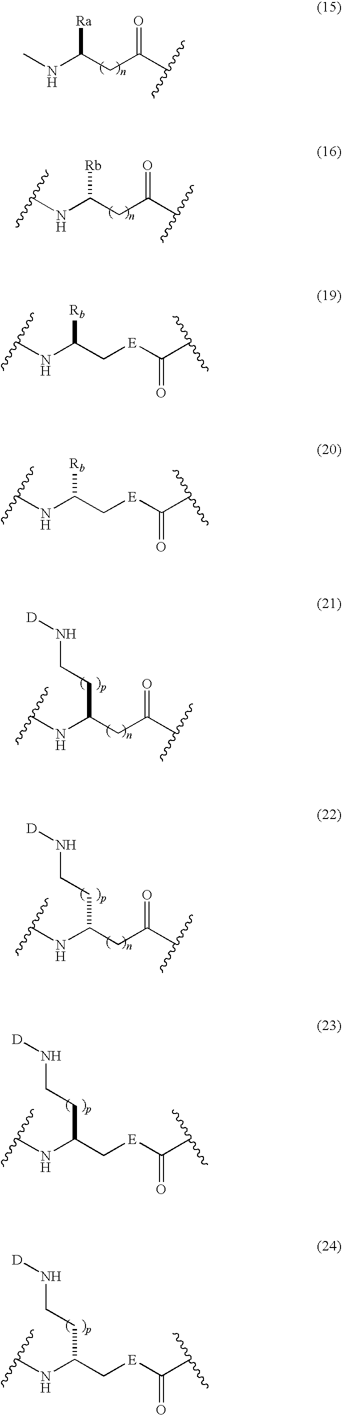 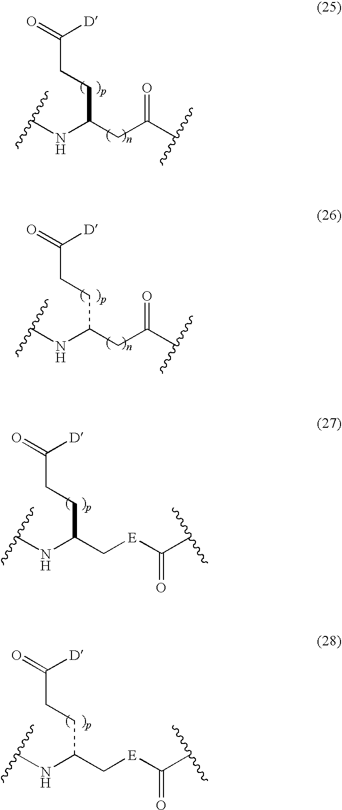
n designating 0, 1, 2 or 3;
p designating 0, 1, 2 or 3;
E designating NH or 0;
R_{b} represents an alkyl chain including from 1 to 6 carbon atoms;
D′ designating one of the following groups: 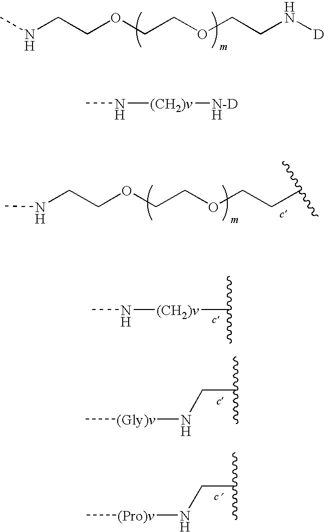
m being an integer ranging from 1 to 40;
v being an integer ranging from 1 to 10;
the bond c′ designating the bond to the X group,
D designating the following groups:
a group of formula 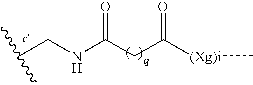
where X represents a group of formulae (13′) and (15′)
or a group of formula 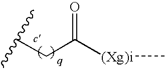
or of formula 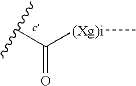
where X represents a group of formulae (1′), (2′), (8′), (9′), (5′), (6′), (7′), (10′), (11′), (12′) and (14′), the c′ bond designating the bond to the X group,
q designating an integer ranging from 2 to 6;
X_{g} corresponding to a residue of one of the following groups:
-(Gly)ₙ-, n ranging from 1 to 10;
-(Pro)ₙ-, n ranging from 1 to 10;
NH₂—(CH₂)ₙ—COON, n ranging from 1 to 10;
NH₂—(CH₂—CH₂—O)ₘ—CH₂CH₂COOH, m ranging from 3 to 6;
8-amino-3,6-dioxaoctanoic acid;
tranexamic acid;
N-methyl-tranexamic acid;
4(piperidin-4-yl)butanoic acid;
3(piperidin-4-yl)propionic acid;
N-(4-aminobutyl)-glycine;
4-carboxymethyl-piperazine;
4-(4-aminophenyl)butanoic acid;
3-(4-aminophenyl)propanoic acid;
4-aminophenylacetic acid;
4-(2-aminoethyl)-1-carboxymethyl-piperazine;
trans-4-aminocyclohexanecarboxylic acid;
cis-4-aminocyclohexanecarboxylic acid;
cis-4-aminocyclohexane acetic acid;
trans-4-aminocyclohexane acetic acid;
4-amino-1-carboxymethyl piperidine;
4-aminobenzoic acid;
4(2-aminoethoxy)benzoic acid.

23. A compound in accordance with claim 17, characterized in that Y has one of the following formulae: 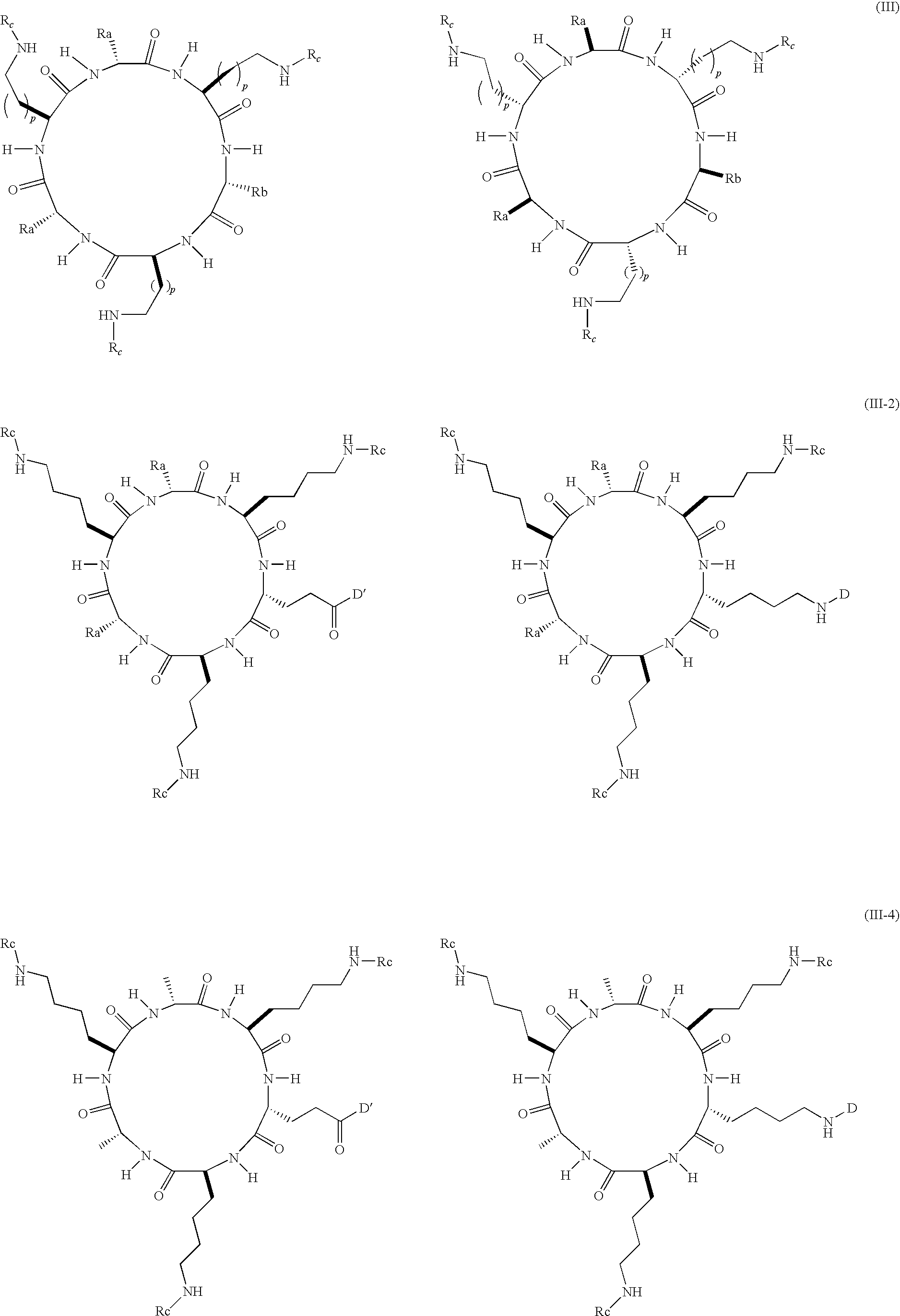 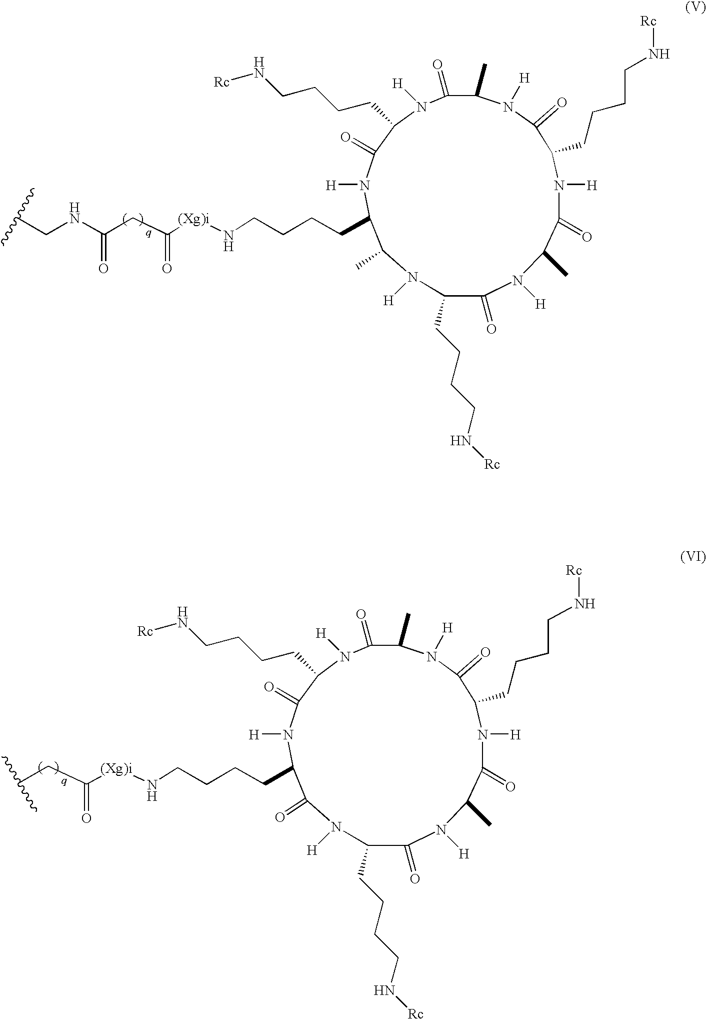
Rₐ, R_{c}, D, D′, X_{g}, q, i and p being as previously defined.

24. A compound in accordance with claim 17, having one of the following formulae (III-a) or (III-b): 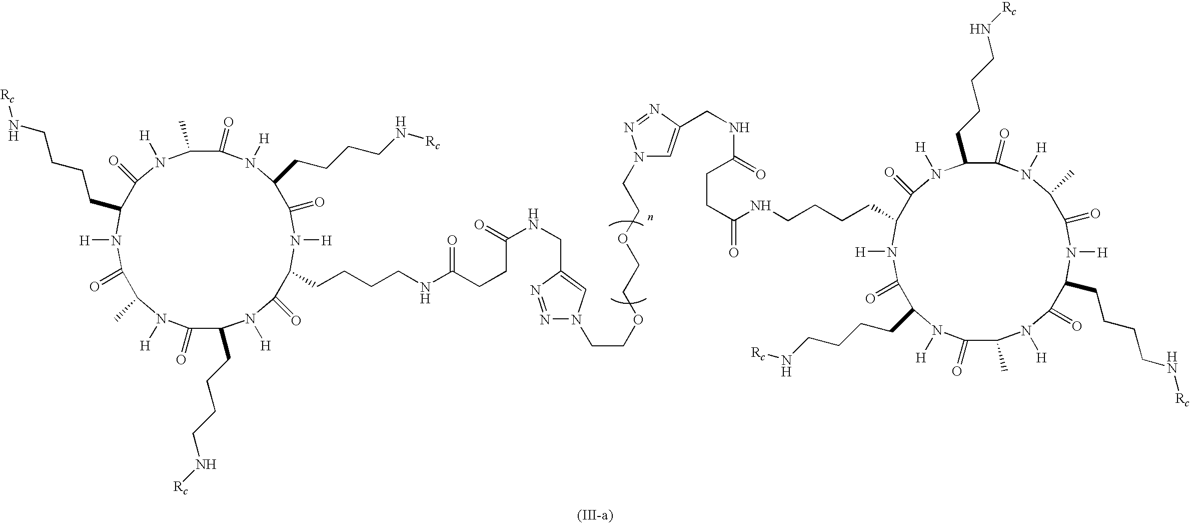 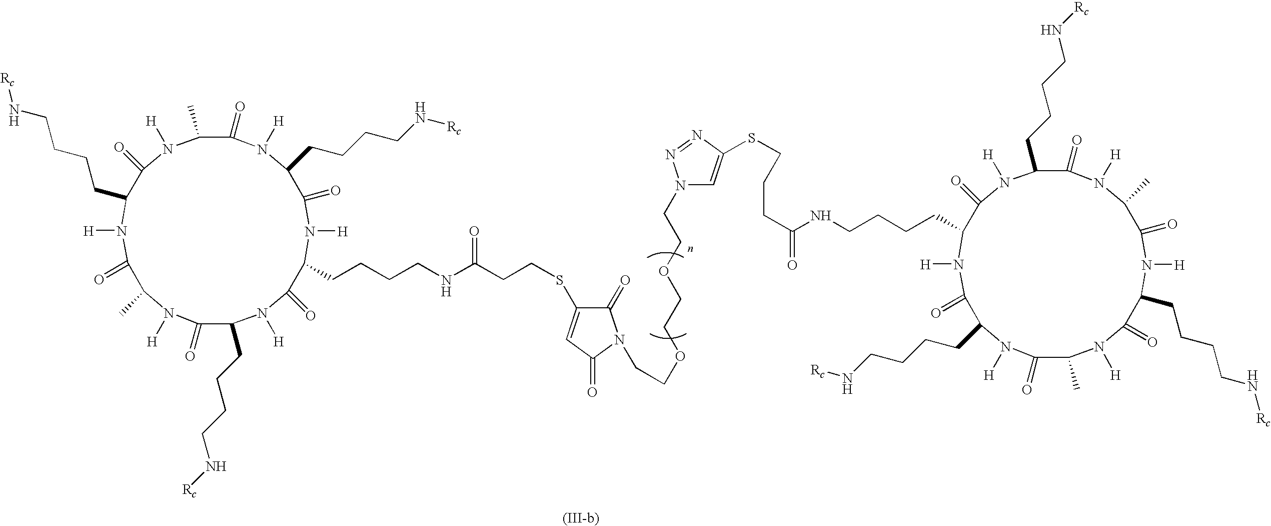
m being as previously defined, and
R_{c} representing:
a) a human or murine CD40 receptor ligand (CD40L)-derived peptide, with said peptide belonging to the primary sequence of the CD40L ligand of CD40 the amino acid number of which is included between 3 and 10, or
b) a peptide belonging to the primary sequence of the CD40L ligand the amino acid number of which is included between 3 and 10 selected among the following sequences: LQWAEKGYYTMSNN (SEQ ID NO: 1); LQWAKKGYYTMKSN (SEQ ID NO: 2); PGRFERILLRAANTH (SEQ ID NO: 3); SIGSERILLKAANTH (SEQ ID NO: 4), or
c) a group of formula H—X, —(X_{b})ₗ—X_{c}—X_{d}—Xₑ—(X_{f})ᵢ— or H—X′ₐ-L-X′_{b}—X_{c}—X_{d}—Xₑ—(X_{f})ᵢ—; wherein
l represents 0 or 1,
i represents 0 or 1,
Xₐ is selected among the following amino acid residues:
lysine;
arginine;
ornithine;
β-amino acids corresponding to lysine, arginine or ornithine bearing the substitution in positions α or β;
tranexamic acid;
N-methyl-tranexamic acid;
8-amino-3,6-dioxaoctanoic acid;
4(piperidin-4-yl)butanoic acid;
3(piperidin-4-yl)propionic acid;
N-(4-aminobutyl)-glycine;
NH₂—(CH₂)ₙ—COON, n ranging from 1 to 10;
NH₂—(CH₂—CH₂—O)ₘ—CH₂CH₂COOH, m ranging from 3 to 6;
4-carboxymethyl-piperazine;
4-(4-aminophenyl)butanoic acid;
3-(4-aminophenyl)propanoic acid;
4-aminophenylacetic acid;
4-(2-aminoethyl)-1-carboxymethyl-piperazine;
trans-4-aminocyclohexanecarboxylic acid;
cis-4-aminocyclohexanecarboxylic acid;
cis-4-aminocyclohexane acetic acid;
trans-4-aminocyclohexane acetic acid;
4-amino-1-carboxymethyl piperidine;
4-aminobenzoic acid;
4(2-aminoethoxy)benzoic acid;
X_{b} is selected among the following amino acid residues:
glycine;
isoleucine;
leucine;
valine;
asparagine;
D-alanine;
D-valine;
L-proline optionally substituted in position β, γ or δ;
D-proline optionally substituted in position β, γ or δ;
N-alkyl-natural amino acids, the alkyl group being a methyl, ethyl or benzyl group;
dialkyl-acyclic amino acids of the following formula: 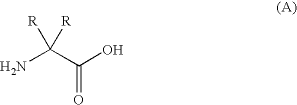
R designating H, Me, Et, Pr or Bu;
dialkyl-cyclic amino acids of the following formula: 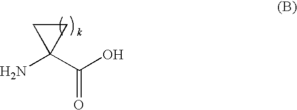
k designating 1, 2, 3 or 4;
X_{c} is selected among the following amino acid residues:
tyrosine;
isoleucine;
leucine;
valine;
phenylalanine;
3-nitro-tyrosine;
4-hydroxymethyl-phenylalanine;
3,5-dihydroxy-phenylalanine;
2,6-dimethyl-tyrosine;
3,4-dihydroxy-phenylalanine (DOPA);
X_{d} is selected among the following amino acid residues:
tyrosine;
isoleucine;
leucine;
valine;
phenylalanine;
3-nitro-tyrosine;
4-hydroxymethyl-phenylalanine;
3,5-dihydroxy-phenylalanine;
2,6-dimethyl-tyrosine;
3,4-dihydroxy-phenylalanine;
Xe is selected among the following amino acid residues:
arginine;
-(Gly)ₙ-, n ranging from 1 to 10;
-(Pro)ₙ-, n ranging from 1 to 10;
NH₂—(CH₂)ₙ—COON, n ranging from 1 to 10;
NH₂—(CH₂—CH₂—O)ₘ—CH₂CH₂COOH, m ranging from 3 to 6;
8-amino-3,6-dioxaoctanoic acid;
tranexamic acid;
N-methyl-tranexamic acid;
4(piperidin-4-yl)butanoic acid;
3(piperidin-4-yl)propionic acid;
N-(4-aminobutyl)-glycine;
4-carboxymethyl-piperazine;
4-(4-aminophenyl)butanoic acid;
3-(4-aminophenyl)propanoic acid;
4-aminophenylacetic acid;
4-(2-aminoethyl)-1-carboxymethyl-piperazine;
trans-4-aminocyclohexanecarboxylic acid;
cis-4-aminocyclohexanecarboxylic acid;
cis-4-aminocyclohexane acetic acid;
trans-4-aminocyclohexane acetic acid;
4-amino-1-carboxymethyl piperidine;
4-aminobenzoic acid;
4(2-aminoethoxy)benzoic acid;
X_{f} is selected among the following amino acid residues:
-(Gly)ₙ-, n ranging from 1 to 10;
-(Pro)ₙ-, n ranging from 1 to 10;
NH₂—(CH₂)ₙ—COON, n ranging from 1 to 10;
NH₂—(CH₂—CH₂—O)ₘ—CH₂CH₂COOH, m ranging from 3 to 6;
8-amino-3,6-dioxaoctanoic acid;
tranexamic acid;
N-methyl-tranexamic acid;
4(piperidin-4-yl)butanoic acid;
3(piperidin-4-yl)propionic acid;
N-(4-aminobutyl)-glycine;
4-carboxymethyl-piperazine;
4-(4-aminophenyl)butanoic acid;
3-(4-aminophenyl)propanoic acid;
4-aminophenylacetic acid;
4-(2-aminoethyl)-1-carboxymethyl-piperazine;
trans-4-aminocyclohexanecarboxylic acid;
cis-4-aminocyclohexanecarboxylic acid;
cis-4-aminocyclohexane acetic acid;
trans-4-aminocyclohexane acetic acid;
4-amino-1-carboxymethyl piperidine;
4-aminobenzoic acid; 4(2-aminoethoxy)benzoic acid;
-L- represents a peptide-like type bond between residues X′ₐ and X′_{b} if X′_{b} represents a proline side chain,
Fₖ and Fₖ' represent, independently from each other, a hydrogen, a halogen, an alkyl group of 1 to 20 carbon atoms, or an aryl group the ring structure of which includes from 5 to 20 carbon atoms,
X′ₐ designates the side chain of lysine, arginine or ornithine; and
X′_{b} designates the side chain of one of the following amino acid residues:
glycine;
isoleucine;
leucine;
valine;
asparagine;
D-alanine;
D-valine;
L-proline optionally substituted in position β, γ or δ;
D-proline optionally substituted in position β, γ or δ;
N-alkyl natural amino acids, the alkyl group of which is a methyl, ethyl or benzyl group;
dialkyl-acyclic amino acids of the following formula: 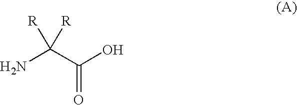
R designating H, Me, Et, Pr or Bu;
dialkyl-cyclic amino acids of the following formula: 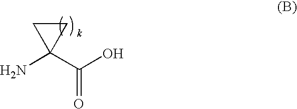
k designating 1, 2, 3 or 4.

25. A compound in accordance with claim 17, wherein R₁ is:
H-Lys-Gly-Tyr-Tyr-NH—(CH₂)₅—CO— or
H-Lys-(D)Pro-Tyr-Tyr-NH—(CH₂)₅—CO— or
Ahx-(D)Pro-Tyr-Tyr-NH—(CH₂)₅—CO— or
H-Lys-ψ(CH₂N)-(D)Pro-Tyr-Tyr-NH—(CH₂)₅—CO—, -ψ(CH₂N)— corresponding to a peptide-like bond of a methylene-amino type, or
H-Lys-ψ(CH₂)-Gly-Tyr-Tyr-NH—(CH₂)₅—CO—, -ψ(CH₂)— corresponding to a peptide-like bond of a methylene type, or
H-Lys-ψ(CH₂—CH₂)-Gly-Tyr-Tyr-NH—(CH₂)₅—CO—, -ψ(CH₂CH₂ corresponding to a peptide-like bond of an ethylene type, or
H-Lys-Gly-DOPA-Tyr-NH—(CH₂)₅—CO—, or
H-Arg-Ile-Ile-Leu-Arg-NH—(CH₂)₅—CO—.

26. A compound in accordance with claim 17, having the following formula (III-a): 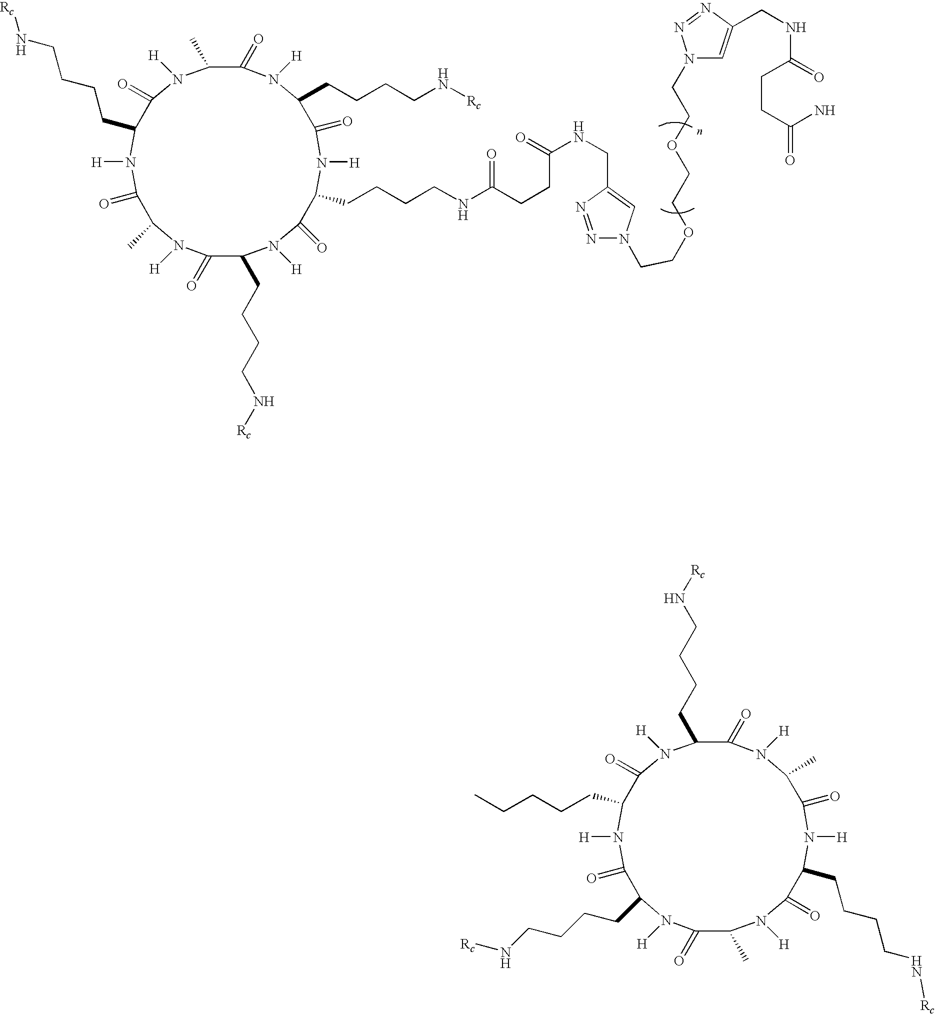
wherein:
n is equal to 1, 2 or 6;
R_{c} represents a H-Lys-Gly-Tyr-Tyr-NH—(CH₂)₅—CO— group.

27. A pharmaceutical composition characterized in that it comprises, as an active ingredient, a compound in accordance with claim 17, in combination with a pharmaceutically acceptable carrier

28. A vaccine composition, characterized in that it includes, as an active ingredient, a compound in accordance with claim 17, in combination with a pharmaceutically acceptable adjuvant.

29. Method for the treatment of pathologies, said treatment involving the inhibition or activation of the immune response, wherein a compound of claim 17 is used.

30. Method for the treatment of pathologies, said treatment involving the inhibition of the immune response wherein a compound of claim 17 is used.

31. Method for the treatment of pathologies, said treatment involving the inhibition of the immune response, wherein the said pathologies are selected in the list consisting in graft rejection, allergic disease or autoimmune diseases, and wherein a compound of claim 17 is used.

32. Method for the treatment of pathologies, said treatment involving the activation of the immune response, wherein a compound of claim 17 is used.

33. Method for the treatment of pathologies, said treatment involving the activation of the immune response, wherein the said pathologies are selected in the list consisting in cancers or parasitic, bacterial, viral or infections involving non conventional infectious agents such as prions, and
wherein a compound of claim 17 is used.

34. A process for preparing on a solid support a compound, said compound having the following formula (I): 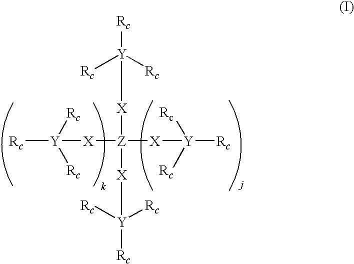
wherein:
k and j represent independently from each other 0 or 1,
Y represents a macrocycle the ring of which includes from 9 to 36 atoms, and is functionally substituted by three amine functionalities and by a carbon chain allowing binding of a Z spacer via an X bond,
R_{c} represents a binding motif to a receptor belonging to the TNF superfamily, and preferably represents a ligand derived-sequence selected among the residues interfacing with the ligand receptor, which sequence may interact with the receptor, said ligand being selected among receptor ligands belonging to the TNF superfamily, namely among the following ligands: EDA, CD40L, FasL, OX40L, AITRL, CD30L, VEGI, LIGHT, 4-1 BBL, CD27L, LTα, TNF, LTβ, TWEAK, APRIL, BLYS, RANKL and TRAIL,
X represents a chemical functionality which allows the Y group to be linked to the spacer and is selected among the following functional groups: 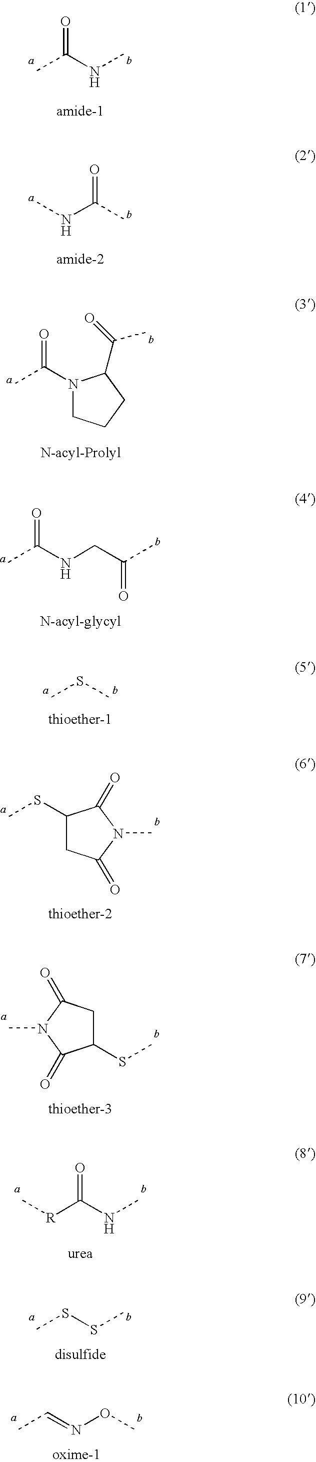 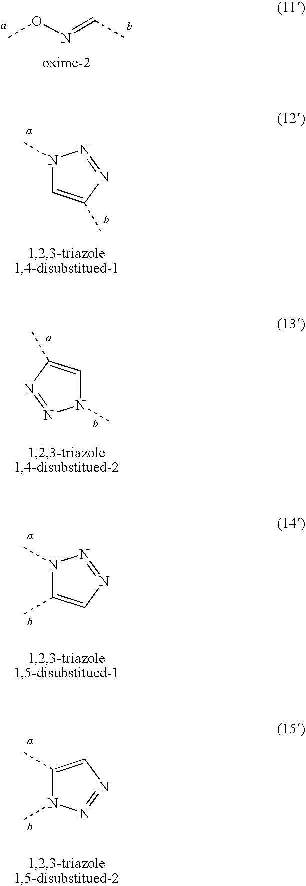
a designating the bond to the Y group and b designating the bond to the Z group,
Z represents a bi, tri- or tetrafunctional spacer having one of the following formulae:
if j=k=0:
if X represents a group of formulae (1′), (8′), (9′), (5′), (6′), (7′), (13′) and (15′), Z has one of the following formulae: 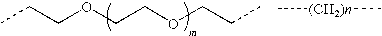
m being an integer ranging from 1 to 40,
n being an integer ranging from 1 to 10,
if X represents a group of formulae (2′), (3′) and (4′), Z has one of the following formulae: 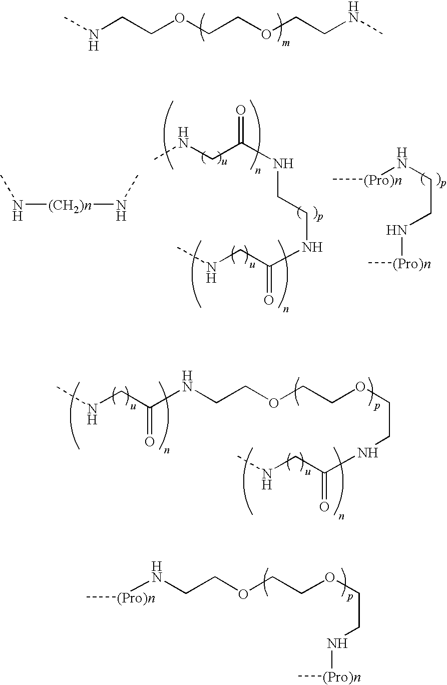
if X represents a group of formulae (12′) and (14′), Z has one of the following formulae: 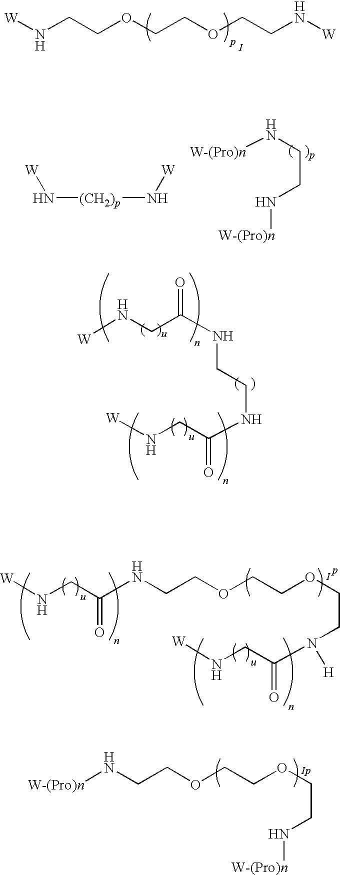
m and n being as defined above,
p being an integer ranging from 1 to 6,
u being an integer ranging from 1 to 4,
W designating a group of formula 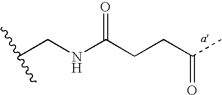
or a group of formula 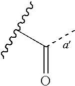
the W group being bound to the NH group through the dotted line bond a′
if X represents a group of formulae (1′), (2′), (8′), (9′), (5′), (6′), (7′), (10′), (11′), (13′) and (15′), Z has one of the following formulae: 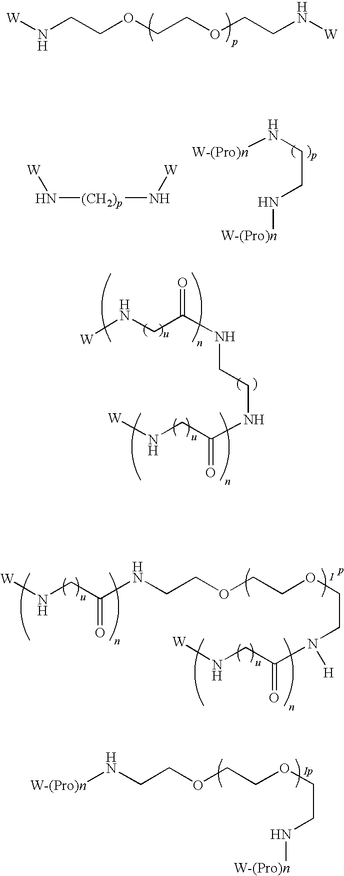
m and n being as defined above,
p being an integer ranging from 1 to 6,
u being an integer ranging from 1 to 4,
W designating a group of formula 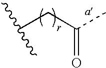
r being an integer ranging from 1 to 4,
the W group being bound to the NH group via the dotted line bond a′
if j=1 or k=1
if X represents a group of formulae (12′) and (14′), Z has one of the following formulae: 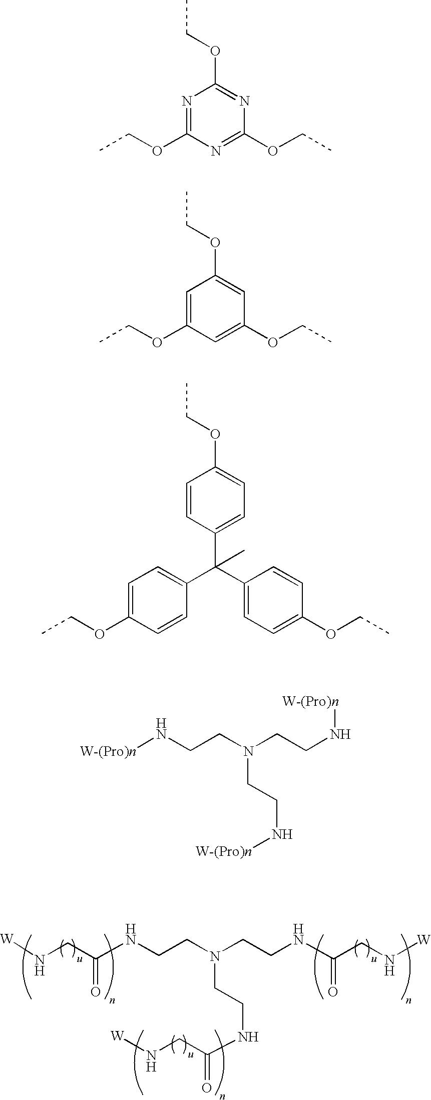
u being an integer ranging from 1 to 4,
n being an integer ranging from 1 to 10,
W designating a group of formula 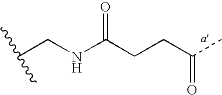
or a group of formula 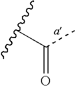
the W group being bound to the NH group through the dotted line bond a′,
if X represents a group of formulae (1′), (2′), (8′), (9′), (5′), (6′), (7′), (10′), (11′), (13′) and (15′), Z has one of the following formulae: 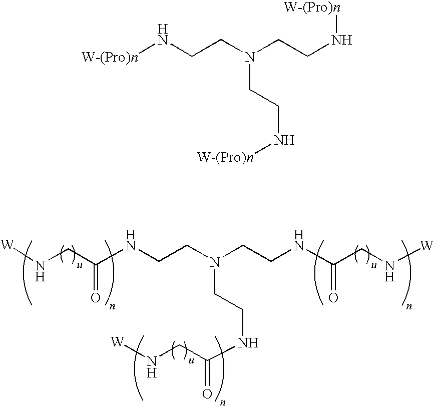
u being an integer ranging from 1 to 4,
n being an integer ranging from 1 to 10,
W designating a group of formula 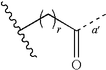
r being an integer ranging from 1 to 4,
the W group being bound to the NH group via the dotted line bond a′
Z can also represent one of the following formulae: 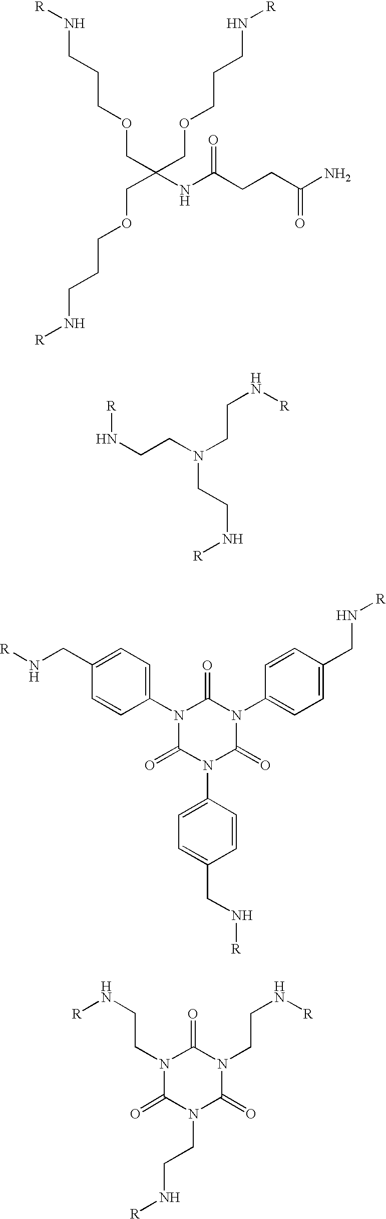 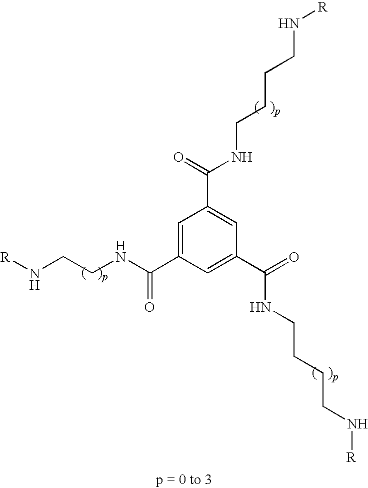
wherein:
if X represents a group of formulae (1′), (8′), (9′), (5′), (6′), (7′), (13′) and (15′):
R represents one of the following groups: 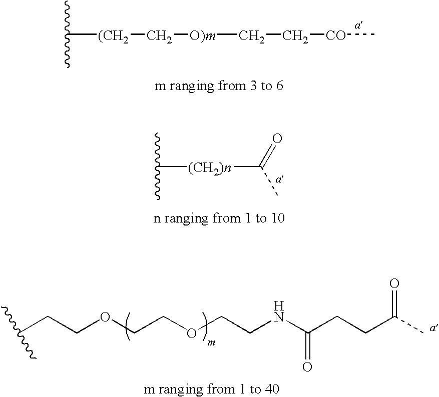
the R group being bound to the NH group via the dotted line bond a′,
if X represents a group of formulae (2′), (3′) and (4′):
R represents one of the following groups: 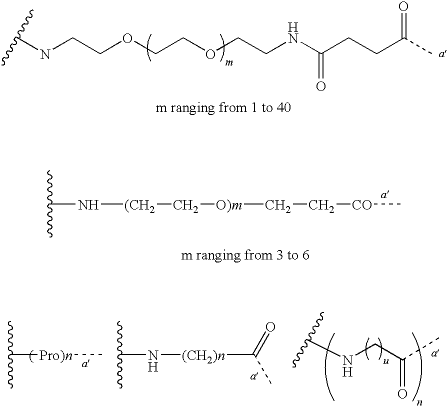
n being an integer ranging from 1 to 10,
u being an integer ranging from 1 to 4,
the R group being bound to the NH group through the dotted line bond a′,
if X represents a group of formulae (12′) and (14′):
R represents one of the following groups: 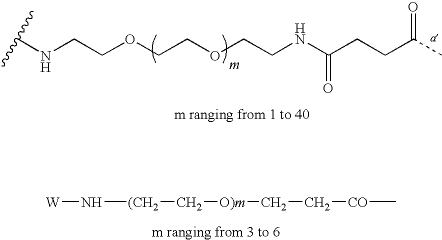
the R group being bound to the NH group through the dotted line bond a′, 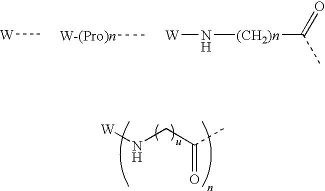
n being an integer ranging from 1 to 10,
W designating a group of formula 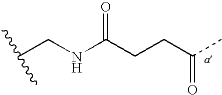
or a group of formula 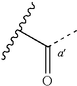
the W group being bound to the NH group through the dotted line bond a′
if X represents a group of formulae (1′), (2′), (8′), (9′), (5′), (6′), (7′), (10′), (11′), (13′) and (15′):
R represents one of the following groups: 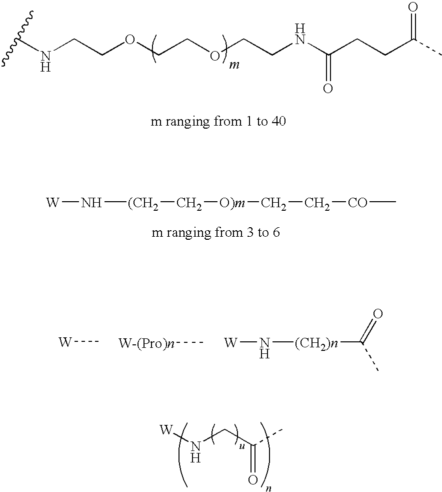
u and n being as specified above,
W designating a group of formula 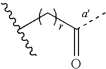
r being an integer ranging from 1 to 4,
the W group being bound to the NH group through the dotted line bond a′,
with said process being characterized in that it comprises the following steps:
forming a linear precursor of Y as specified above, which precursor is constituted of an amino acid sequence forming a growing peptide chain, being synthesized by successive coupling cycles of N-protected amino acid residues, three of which bear an amine type group, and the amine functionality of the growing peptide chain, and deprotecting, the first amino acid residue being bound to a solid support, said linear precursor of Y comprising at least one D-alanine residue, said D-alanine residue being substituted by a D-lysine residue the e-NH amine of which has been acylated by a carboxylic acid bearing the desired functionality corresponding to an X group as specified above,
cyclizing the linear precursor of Y in a protected state mentioned above, in order to obtain a protected functionally substituted ring,
reacting said protected functionally substituted ring with a Z-derived spacer as specified above, in order to obtain a dimerized protected functionally substituted ring,
cleaving said previously mentioned protecting groups, to free said previously mentioned protected amine functionalities and obtain a dimerized deprotected functionally substituted ring,
coupling said previously mentioned freed amine functionalities with an in situ formed or preformed peptide by successive assembly of amino acid residues corresponding to the R_{c} group as specified above, and
cleaving the molecule from the solid support, after removal of all protecting groups present on functionally substituted side chains of the R_{c} group, in order to obtain the said compound.

35. A process for preparing in accordance with claim 34, a compound of formula (III-a) or (III-b), wherein the Rc group of said compound is:
H-Lys-Gly-Tyr-Tyr-NH—(CH₂)₅—CO— or
H-Lys-(D)Pro-Tyr-Tyr-NH—(CH₂)₅—CO— or
Ahx-(D)Pro-Tyr-Tyr-NH—(CH₂)₅—CO— or
H-Lys-ψ(CH₂N)-(D)Pro-Tyr-Tyr-NH—(CH₂)₅—CO—, -ψ(CH₂N)— corresponding to a peptide-like bond of a methylene-amino type, or
H-Lys-ψ(CH₂)-Gly-Tyr-Tyr-NH—(CH₂)₅—CO—, -ψ(CH₂)— corresponding to a peptide-like bond of a methylene type, or
H-Lys-ψ(CH₂—CH₂)-Gly-Tyr-Tyr-NH—(CH₂)₅—CO—, -ψ(CH₂CH₂ corresponding to a peptide-like bond of an ethylene type, or
H-Lys-Gly-DOPA-Tyr-NH—(CH₂)₅—CO—, or
H-Arg-Ile-Ile-Leu-Arg-NH—(CH₂)₅—CO—,
said process being characterized in that it includes the following steps:
reacting a protected functionally substituted ring of the following formula: 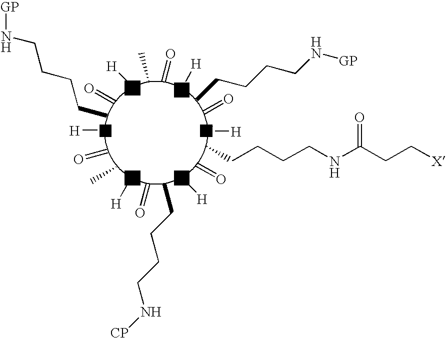
GP denoting a protecting group,
X′ denoting a —SH group or 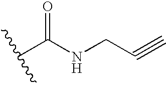
with a spacer group of formula 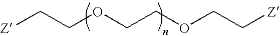
n denoting an integer ranging from 1 to 10,
Z denoting a N₃ group or a group 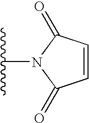
it being understood that when X′ represents a group 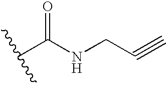
Z′ represents a N₃ group,
and when X′ represents a —SH group, Z′ represents a group 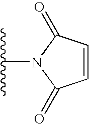
in order to obtain a compound of the following formula (VIII): 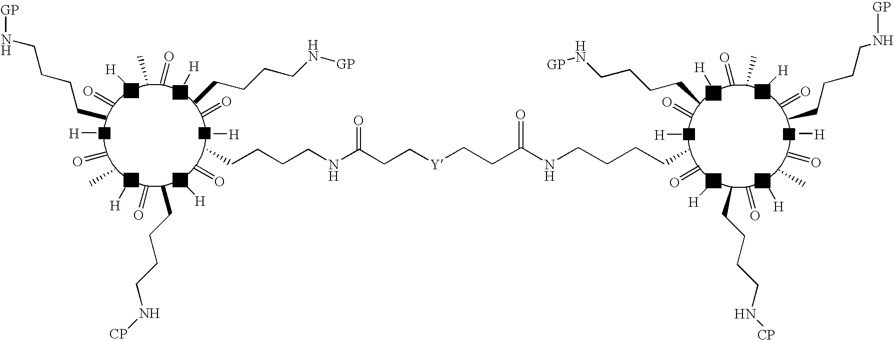
GP being as specified above and
Y′ having one of the formulae given below: 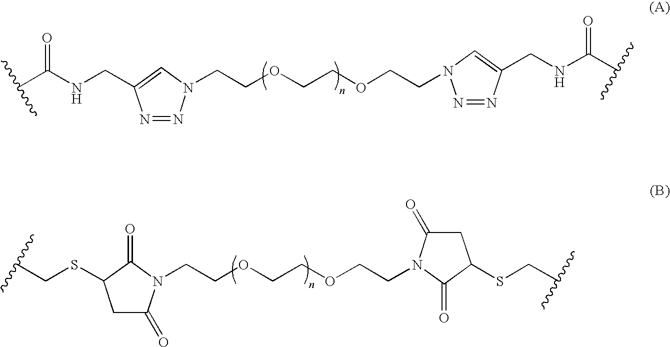
it being understood that Y′ has formula (A) if Z′ represents a N₃ group
and that Y′ has formula (B) if Z′ represents a group 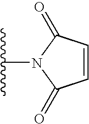
deprotecting said previously mentioned protecting groups GP, to free protected amine functionalities and coupling said previously mentioned freed amine functionalities to an in situ formed or preformed peptide by successive assembly of corresponding amino acid residues to the R_{c} group as specified above, and
cleaving the molecule from its solid support, following removal of all protecting groups present on functionally substituted side chains of the R_{c} group, in order to obtain the said compound of formula (III-a) or (IIIb).

36. A Process according to claim 35, wherein the protecting group GP is selected in the list consisting of Boc, Z or Alloc.
